(19) United States
(12) Patent Application Publication
(10) Pub. No.: US 2011/0009460 A1
(43) Pub. Date: Jan. 13, 2011

(54) COMPOSITIONS AND METHODS FOR TREATING AMYOTROPHIC LATERAL SCLEROSIS

(76) Inventors: Valentin GRIBKOFF, Wallingford, CT (US); Michael E. BOZIK, Pittsburgh, PA (US)

Correspondence Address: Honigman Miller Schwartz and Cohn LLP/BIIB, 350 East Michigan Avenue, Suite 300, Kalamazoo, MI, 49007-3800, US

(21) Appl. No.: 12/819,990

(22) Filed: Jun. 21, 2010

Related U.S. Application Data
(60) Provisional application No. 61/218,659, filed on Jun. 19, 2009.
(60) Provisional application No. 61/267,945, filed on Dec. 9, 2009.
(60) Provisional application No. 61/317,118, filed on Mar. 24, 2010.
(60) Provisional application No. 61/356,439, filed on Jun. 18, 2010.

(51) Int. Cl. A61K 31/428; A61P 25/00

(52) U.S. Cl. 514/367

(57) ABSTRACT

Pharmaceutical compositions of dexpramipexole and methods of using such compositions for the treatment of ALS are disclosed.

### CROSS REFERENCE

This application claims the benefit of U.S. Provisional Application Ser. No. 61/218,659, filed Jun. 19, 2009, U.S. Provisional Application Ser. No. 61/267,945, filed Dec. 9, 2009, U.S. Provisional Application Ser. No. 61/317,118, filed Mar. 24, 2010, and U.S. Provisional Application No. 61/356,439 filed on Jun. 18, 2010 each of which is incorporated herein by reference in its entirety.

### GOVERNMENT INTERESTS

Not applicable.

### PARTIES TO A JOINT RESEARCH AGREEMENT

Not applicable.

### INCORPORATION BY REFERENCE OF MATERIAL SUBMITTED ON A COMPACT DISC

Not applicable.

### BACKGROUND

Not applicable.

### SUMMARY OF THE INVENTION

Various embodiments described herein are directed to a method for treating amyotrophic lateral sclerosis (ALS) in a patient including the step of administering to the patient an effective amount of about chirally pure (6R)-2-amino-4,5,6,7-tetrahydro-6-(propylamino)benzothiazole or pharmaceutically acceptable salt thereof. In some embodiments, treating can include slowing progression of amyotrophic lateral sclerosis (ALS), reducing intensity of symptoms associated with amyotrophic lateral sclerosis (ALS), reducing onset of symptoms associated with amyotrophic lateral sclerosis (ALS), reducing weight loss associated with amyotrophic lateral sclerosis (ALS), reversing weight loss associated with amyotrophic lateral sclerosis (ALS), delaying mortality, and combinations thereof. In particular embodiments, the symptoms associated with amyotrophic lateral sclerosis (ALS) may be, for example, fine motor function, gross motor function, balbar function, respiratory function, and combinations thereof, and in other embodiments, the symptoms associated with amyotrophic lateral sclerosis (ALS) can include walking, speech, eating, swallowing, writing, climbing stairs, cutting food, turning in bed, salivation, dressing, maintaining hygiene, breathing, dyspnea, orthopnea, respiratory insufficiency, and combinations thereof.

In some embodiments, the effective amount may be from about 50 mg to about 300 mg per day, and in other embodiments, the effective amount may be from about 150 mg to about 300 mg per day. In still other embodiments, the effective amount may be about 300 mg or more per day. In certain embodiments, the effective amount may be a stable daily dose. In some embodiments, the stable daily dose may be from about 50 mg to about 300 mg of about chirally pure (6R)-2-amino-4,5,6,7-tetrahydro-6-(propylamino)benzothiazole or pharmaceutically acceptable salt thereof. In other embodiments, the stable daily dose may be 1 to 5 unit doses per day, and in particular embodiments, each unit dose may be a solid unit dose. In some embodiments, administering may include administering one unit dose two times per day wherein each unit dose is equal to about half of the stable daily dose, and in other embodiments, administering may include administering one unit dose once every 12 hours wherein each unit dose is equal to about half of the stable daily dose. In still other embodiments, administering may include administering one unit dose four times per day wherein each unit dose is equal to about one quarter of the stable daily dose. In yet other embodiments, administering can include administering two unit doses wherein each unit dose is about 150 mg two times per day, and in further embodiments, administering may include administering four unit doses wherein each unit dose is about 75 mg four times per day.

In some embodiments, the method may further include the step of monitoring the patient, and in particular embodiments, the method may include the step of monitoring the patient for neutropenia. In other embodiments, monitoring may be ALSFRS-R score for the patient or monitoring the patients fine motor function, gross motor function, bulbar function, respiratory function, and combinations thereof. In still other embodiments, the method may include monitoring behaviors selected from the group consisting of swallowing, handwriting, speech, ability to walk, ability to climb stairs, ability to dress, ability to maintain hygiene, and combinations thereof. In some embodiments, the method may include scheduling a doctor visit every 6 months for at least 12 months.

In certain embodiments, the patient may be predisposed to amyotrophic lateral sclerosis (ALS) and is not exhibiting symptoms of amyotrophic lateral sclerosis (ALS). In some embodimentns, the method may include administering about chirally pure (6R)-2-amino-4,5,6,7-tetrahydro-6-(propylamino)benzothiazole or pharmaceutically acceptable salt thereof to family members of the patient. In other embodiments, the method may include administering the about chirally pure (6R)-2-amino-4,5,6,7-tetrahydro-6-(propylamino)benzothiazole or pharmaceutically acceptable salt thereof to a patient not exhibiting symptoms of amyotrophic lateral sclerosis (ALS), and in further embodiments, the method may include administering the about chirally pure (6R)-2-amino-4,5,6,7-tetrahydro-6-(propylamino)benzothiazole or pharmaceutically acceptable salt thereof to a patient that is predisposed to amyotrophic lateral sclerosis (ALS).

### DESCRIPTION OF THE DRAWINGS

FIG. 1 is a bar graph showing the mean change ALSFRS-R score by subdomain.

FIG. 2 shows the change from baseline in for vital capacity (VC) for treatment groups.

FIG. 3 shows thechange from baseline in ALSFRS-R for treatment groups.

FIG. 4A-C show plots of the mean change in ALSFRS-R score over time and bar graphs of the mean change in baseline based on individual fine motor behaviors, handwriting (FIG. 4A), cutting food (FIG. 4B), and dressing and hygiene (FIG. 4C), tested in ALSFRS-R.

FIG. 5A-C show plots of the mean change in ALSFRS-R score over time and bar graphs of the mean change in baseline based on individual bulbar domain functions, swallowin (FIG. 5A), speech (FIG. 5B), and salivation (FIG. 5C), tested in ALSFRS-R.

FIG. 6A-C show plots of the mean change in ALSFRS-R score over time and bar graphs of the mean change in baseline based on individual gross motor behaviors, turning in bed (FIG. 6A), walking (FIG. 6B), and climbing stairs (FIG. 6C), tested in ALSFRS-R.

FIG. 7A-C show plots of the mean change in ALSFRS-R score over time and bar graphs of the mean change in baseline based on individual respiratory functions, dyspnea (FIG. 7A), orthopnea (FIG. 7B), and respiratory insufficiency (FIG. 8C), tested in ALSFRS-R.

FIG. 8 shows bar graphs illustrating the change from basline in ALSFRS-R score by question for Part 1 and Part 2.

FIG. 9 shows box plots of change from baseline in ALSFRS-R for treatment groups.

FIG. 10 shows the change in ALSFRS-R from baseline to end for each treatment group.

FIG. 11 is a bar graph showing the change from baseline in ALSFRS-R for placebo and the 300 mg treatment group.

FIG. 12 is a schematic of Part 1 and Part 2 of the study.

FIG. 13 shows Kaplan-Meier Estimates for Time to Tracheostomy or Death—Double-Blind Treatment Period (Safety Population).

FIG. 14 show a plot of Mean (SE) ALSFRS-R Total Scores Estimated from the Linear Mixed-Effects Model for Slope (horizontal axis is weeks of active treatment starting at the Part 2, Week 4 visit).

FIG. 15 shows a graphic presentation of Kaplan-Meier Estimates for Time to Death (Double-Blind Treatment Period through Week 28).

FIG. 16 shows a Plot of Mean (SE) Rank of Joint Scores for Combined Time to Death and Changes from Baseline in ALSFRS-R Total Scores (double-blind treatment period through Week 28).

FIG. 17 shows a plot of Mean (SE) ALSFRS-R Total Score Estimates from the Linear Mixed Effects Model for Slope Including Imputed Values of Zero for the First Post-death Visit among Subjects who Died (Double-Blind Treatment Period through Week 28).

FIG. 18 shows a plot of Mean (SE) from Linear Mixed Effects Model Estimates for the Slope of Upright Vital Capacity (with imputed zeroes for the first post-death visit among subjects who died—time from first dose in double-blind treatment period through Week 28).

FIG. 19 shows Kaplan-Meier estimates for time to feeding tube placement—double-blind treatment period (safety population).

FIG. 20 shows the Kaplan-Meier Estimates for time to tracheotomy or death-double blind treatment period (safety population).

FIG. 21 shows the mean plasma dexpramipexole concentration after oral administration of single 50 mg, 150 mg, and 300 mg doses under fasted conditions-linear axis.

FIG. 22 shows the mean plasma dexpramipexole concentration after oral administration of a single 150 mg dose under fasted and fed conditions-linear axis.

FIG. 23 shows the mean plasma dexpramipexole concentration on day 7 after oral administration of single 50 mg, 150 mg, and 300 mg doses on day 1, twice daily doses on day 3 through 6 and single doses on day 7 under fasted conditions-linear axis.

FIG. 24 shows the mean positional changes in systolic and diastolic blood pressures (standing minus supine) following 4½ days of multiple doses of dexpramipexole or placebo.

### DETAILED DESCRIPTION

Before the present compositions and methods are described, it is to be understood that this invention is not limited to the particular processes, compositions, or methodologies described, as these may vary. Moreover, the processes, compositions, and methodologies described in particular embodiments are interchangeable. Therefore, for example, a composition, dosage regimen, route of administration, and so on described in a particular embodiments may be used in any of the methods described in other particular embodiments. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art. Although any methods similar or equivalent to those described herein can be used in the practice or testing of embodiments of the present invention, the preferred methods are now described. All publications and references mentioned herein are incorporated by reference. Nothing herein is to be construed as an admission that the invention is not entitled to antedate such disclosure by virtue of prior invention.

It must be noted that, as used herein, and in the appended claims, the singular forms “a”, “an” and “the” include plural reference unless the context clearly dictates otherwise.

Embodiments including the transition phrase “consisting of” or “consisting essentially of” include only the recited components and inactive ingredients. For example, a composition “consisting essentially of” dexpramipexole can include dexpramipexole and inactive excipients, which may or may not be recited, but may not contain any additional active agents or neuroprotectants. A composition “consisting of” dexpramipexole may include only the components specifically recited.

As used herein, the term “about” means plus or minus 10% of the numerical value of the number with which it is being used. Therefore, about 50% means in the range of 45%-55%.

“Optional” or “optionally” may be taken to mean that the subsequently described structure, event or circumstance may or may not occur, and that the description includes instances where the event occurs and instances where it does not.

“Administering” when used in conjunction with a therapeutic means to administer a therapeutic directly into or onto a target tissue or to administer a therapeutic to a patient whereby the therapeutic positively impacts the tissue to which it is targeted. “Administering” a composition may be accomplished by oral administration, injection, infusion, absorption or by any method in combination with other known techniques. “Administering” may include the act of self administration of administration by another person such as a health care provider or a device.

The term “improves” is used to convey that the present invention changes either the appearance, form, characteristics and/or physical attributes of the tissue to which it is being provided, applied or administered. “Improves” may also refer to the overall physical state of an individual to whom an active agent has been administered. For example, the overall physical state of an individual may “improve” if one or more symptoms of a neurodegenerative disorder are alleviated by administration of an active agent.

As used herein, the term “therapeutic” means an agent utilized to treat, combat, ameliorate or prevent an unwanted condition or disease of a patient.

The terms “therapeutically effective amount” or “therapeutic dose” as used herein are interchangeable and may refer to the amount of an active agent or pharmaceutical compound or composition that elicits a biological or medicinal response in a tissue, system, animal, individual or human that is being sought by a researcher, veterinarian, medical doctor or other clinician. A biological or medicinal response may include, for example, one or more of the following: (1) preventing a disease, condition or disorder in an individual that may be predisposed to the disease, condition or disorder but does not yet experience or display pathology or symptoms of the disease, condition or disorder, (2) inhibiting a disease, condition or disorder in an individual that is experiencing or displaying the pathology or symptoms of the disease, condition or disorder or arresting further development of the pathology and/or symptoms of the disease, condition or disorder, and (3) ameliorating a disease, condition or disorder in an individual that is experiencing or exhibiting the pathology or symptoms of the disease, condition or disorder or reversing the pathology and/or symptoms experienced or exhibited by the individual.

As used herein, the term “neuroprotectant” refers to any agent that may prevent, ameliorate or slow the progression of neuronal degeneration and/or neuronal cell death.

The term “treating” may be taken to mean prophylaxis of a specific disorder, disease or condition, alleviation of the symptoms associated with a specific disorder, disease or condition and/or prevention of the symptoms associated with a specific disorder, disease or condition. In some embodiments, the term refers to slowing the progression of the disorder, disease or condition or alleviating the symptoms associated with the specific disorder, disease or condition. In some embodiments, the term refers to slowing the progression of the disorder, disease or condition. In some embodiments, the term refers to alleviating the symptoms associated with the specific disorder, disease or condition. In some embodiments, the term refers to restoring function which was impaired or lost due to a specific disorder, disease or condition.

The term “patient” generally refers to any living organism to which compounds described herein are administered and may include, but is not limited to, any non-human mammal, primate or human. Such “patients” may or my not be exhibiting the signs, symptoms or pathology of the particular diseased state.

As used herein, the term “naïve patient” refers to a patient that has not previously received pramipexole treatment (either (R)-pramipexole or (S)-pramipexole), particularly, (R)-pramipexole, or who has not received a titration regimen of pramipexole previous to receiving a starting dose of pramipexole.

As used herein, the terms “enantiomers,” “stereoisomers,” and “optical isomers” may be used interchangeably and refer to molecules which contain an asymmetric or chiral center and are mirror images of one another. Further, the terms “enantiomers,” “stereoisomers,” or “optical isomers” describe a molecule which, in a given configuration, cannot be superimposed on its mirror image.

As used herein, the terms “optically pure” or “enantiomerically pure” may be taken to indicate that a composition contains at least 99.95% of a single optical isomer of a compound. The term “enantiomerically enriched” may be taken to indicate that at least 51% of a composition is a single optical isomer or enantiomer. The term “enantiomeric enrichment” as used herein refers to an increase in the amount of one enantiomer as compared to the other. A “racemic” mixture is a mixture of about equal amounts of (6R) and (6S) enantiomers of a chiral molecule.

Throughout this disclosure, the word “pramipexole” will refer to (6S) enantiomer of 2-amino-4,5,6,7-tetrahydro-6-(propylamino)benzothiazole unless otherwise specified.

The term “pharmaceutical composition” shall mean a composition including at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan. A pharmaceutical composition may, for example, contain dexpramipexole or a pharmaceutically acceptable salt of dexpramipexole as the active ingredient. Alternatively, a pharmaceutical composition may contain dexpramipexole or a pharmaceutically acceptable salt of dexpramipexole as the active ingredient.

For the purposes of this disclosure, a “salt” is any acid addition salt, preferably a pharmaceutically acceptable acid addition salt, including but not limited to, halogenic acid salts such as hydrobromic, hydrochloric, hydrofluoric and hydroiodic acid salt; an inorganic acid salt such as, for example, nitric, perchloric, sulfuric and phosphoric acid salt; an organic acid salt such as, for example, sulfonic acid salts (methanesulfonic, trifluoromethan sulfonic, ethanesulfonic, benzenesulfonic or p-toluenesulfonic), acetic, malic, fumaric, succinic, citric, benzoic, gluconic, lactic, mandelic, mucic, pamoic, pantothenic, oxalic and maleic acid salts; and an amino acid salt such as aspartic or glutamic acid salt. The acid addition salt may be a mono- or di-acid addition salt, such as a di-hydrohalogenic, di-sulfuric, di-phosphoric or di-organic acid salt. In all cases, the acid addition salt is used as an achiral reagent which is not selected on the basis of any expected or known preference for interaction with or precipitation of a specific optical isomer of the products of this disclosure.

“Pharmaceutically acceptable salt” is meant to indicate those salts which are, within the scope of sound medical judgment, suitable for use in contact with the tissues of a patient without undue toxicity, irritation, allergic response and the like, and are commensurate with a reasonable benefit/risk ratio. Pharmaceutically acceptable salts are well known in the art. For example, Berge et al. (1977) J. Pharm. Sciences, Vol 6. 1-19, describes pharmaceutically acceptable salts in detail.

As used herein, the term “daily dose amount” refers to the amount of pramipexole per day that is administered or prescribed to a patient. This amount can be administered in multiple unit doses or in a single unit dose, in a single time during the day or at multiple times during the day.

A “dose amount” or “dose” as used herein, is generally equal to the dosage of the active ingredient which may be administered per day. For example, a dose amount of dexpramipexole may be 150 mg/day or 300 mg/day.

The term “unit dose” as used herein may be taken to indicate a discrete amount of the therapeutic composition that contains a predetermined amount of the active compound. The amount of the active compound is generally equal to the dosage of the active ingredient which may be administered on or more times per day. The unit dose may be a fraction of the desired daily dose which may be given in fractional increments, such as, for example, one-half or one-third the dosage. For example, a 150 mg/day dose amount of dexpramipexole may be administered as 2 unit doses of 75 mg each, 3 unit doses of 50 mg or 4 unit doses of 37.5 mg.

Throughout the application, the term “dopaminergic activity equivalent” (DAE) will be referred to which means the measure of activity at the dopamine receptors equivalent to the activity of 1 mg of pramipexole at the dopamine receptors. For example, a dosage of dexpramipexole having a DAE of 0.01 would have activity at the dopamine receptors which is equivalent to the activity of 0.01 mg of pramipexole. The DAE can also be related to a variety of pharmaceutical terms, including maximum tolerated dose (MTD), no observable adverse effect level (NOAEL), and non-effective dose amount for the sake of clarity. For example, the NOAEL dose amount for pramipexole is most preferably below 0.05 mg. This, in turn, corresponds to a DAE of below 0.05. A dose amount of dexpramipexole having a DAE of 0.01 would, therefore, be below the DAE for the most preferable pramipexole NOAEL dose amount of 0.05 mg. In some embodiments, DAE is determined by measuring the binding affinity (IC₅₀) or activity (EC₅₀) at the D₂ and/or D₃ receptors relative to the same parameter for 1 mg of pramipexole.

The degree to which dosing of a molecule has demonstrable phenotypic activity resulting from affinity to particular receptors or other pharmaco-effective proteins, even when the activity results from affinities to unknown targets, can be operationally defined in terms of whether this activity contributes in a positive way (“on-target” activity) or a negative way (“off-target” activity) to a specific and desired therapeutic effect. For any given molecule, a number of “off-target” activities can theoretically be identified, but “on-target” activity is restricted to the desired therapeutic effect. To the extent that these activities can be measured and quantified, or comparisons be made with known standards, an index of activity can be generated for each of these categories (the “activity equivalent”, or “AE”), and one or more ratios generated to compare “off-target” to “on-target” activities, useful to compare potential risk-benefit ratios between molecules.

Dexpramipexole ((6R)-2-amino-4,5,6,7-tetrahydro-6-(propylamino)benzothiazole) is a synthetic aminobenzothiazole derivative. The (6S) enantiomer of dexpramipexole, commonly known as pramipexole and commercially available under the Mirapex® name, is a potent dopamine agonist, which mimics the effects of the neurotransmitter dopamine. Pramipexole has also been shown to have both neuroprotective and dopaminergic activities, presumably through inhibition of lipid peroxidation, normalization of mitochondrial metabolism and/or detoxification of oxygen radicals. Therefore, pramipexole may have utility as an inhibitor of the cell death cascades and loss of cell viability observed in neurodegenerative diseases such as Parkinson's disease. Additionally, oxidative stress caused by an increase in oxygen and other free radicals has been associated with the fatal neurodegenerative disorder amyotrophic lateral sclerosis (ALS), a progressive neurodegenerative disorder involving the motor neurons of the cortex, brain stem, and spinal cord.

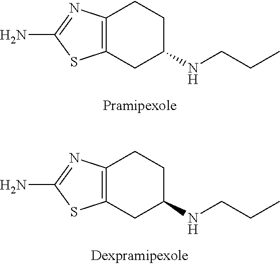

The neuroprotectant activity of both enantiomers are expected to require therapeutic doses in the range of about 10 mg/day to about 1,500 mg/day while pramipexole's agonistic effect on the D₂ family of dopamine receptors only allows therapeutic doses that range between 0.5 and 5.0 mg/day. However, even these low doses significant adverse side effects have been reported. For example, the Boehringer Ingelheim product insert for Mirapex® sets the maximally tolerated dose for humans at 4.5 mg/day, and a dose of pramipexole as low as 1.5 mg has been shown to cause somnolence in humans. Single dose toxicity of pramipexole after oral administration has been studied in rodents, dogs, monkeys and humans. In rodents, death occurred at doses of 70-105 mg/kg and above which is equivalent to a human dose of 7-12 mg/kg or approximately 500-850 mg for a 70 kg (˜150 lb) individual. In dogs, vomiting occurred at 0.0007 mg/kg and above, while monkeys displayed major excitation at 3.5 mg/kg. In human subjects, an initial single dose of pramipexole of greater than 0.20 mg was not tolerated. All species showed signs of toxicity related to exaggerated pharmacodynamic responses to the dopaminergic agonism of pramipexole.

Thus, a clinical use of pramipexole as a mitochondria-targeted neuroprotectant is unlikely, as the high doses needed for the neuroprotective or anti-oxidative/mitochondrial normalization action are not accessible due to high dopamine receptor affinity associated with the (6S) enantiomer. In contrast, (6R)-2-amino-4,5,6,7-tetrahydro-6-(propylamino)benzothiazole (“dexpramipexole”) is an effective mitochondria-targeted agent that exhibits excellent neuroprotective properties when administered without adverse side effects. Additionally, the functional affinity difference between the pramipexole and dexpramipexole (e.g. 10,000-20,000 fold) for dopamine receptor is much greater than previously reported. Thus, higher doses of dexpramipexole can be tolerated by patients and will allow greater brain, spinal cord and mitochondrial concentrations increasing the degree to which oxidative stress and/or mitochondrial dysfunction may be reduced. The neuroprotective effect of dexpramipexole may occur by at least one of three mechanisms. First, dexpramipexole may be capable of reducing the formation of reactive oxygen species in cells with impaired mitochondrial energy production. Second, dexpramipexole may partially restore the reduced mitochondrial membrane potential that is correlated with Alzheimer's, Parkinson's, Huntington's and amyotrophic lateral sclerosis diseases. Third, dexpramipexole may block or attenuate the apoptotic cell death pathways which are produced by pharmacological models of Alzheimer's disease, Parkinson's disease, Huntington's disease, amyotrophic lateral sclerosis diseases and mitochondrial impairment. High doses of dexpramipexole required to elicit these neuroprotective effects generally require highly pure preparations of dexpramipexole which take into account the upper limit of (6S) enantiomer contamination (0.5 mg to 5.0 mg).

Embodiments of the invention are generally directed to pharmaceutical compositions including an effective amount of dexpramipexole and methods for using such pharmaceutical compositions for the treatment of neurological diseases such as, for example, amyotrophic lateral sclerosis (ALS). In particular, embodiments of the invention are directed to methods for treating neurological diseases including the step of administering at least about 150 mg of dexpramipexole per day to a patient in need of treatment, and in other embodiments, at least about 300 mg of dexpramipexole may be administered to a patient in need of treatment per day. Such administration may be carried out as a single dose once per day, or in certain embodiments, two or more doses of dexpramipexole may be administered two or more times per day. Therefore, embodiments of the invention are also directed to pharmaceutical compositions at least including 50 mg of dexpramipexole and a pharmaceutically acceptable excipient, and in some embodiments, such pharmaceutical compositions may include at least 75 mg, 100 mg, 125 mg, 150 mg, 300 mg, 400 mg, 500 mg, or 600 mg of dexpramipexole and one or more pharmaceutically acceptable excipients, which may be administered as described above. In certain embodiments, ALS may be limb-onset ALS or bulbar-onset ALS.

In various embodiments, dexpramipexole administered or incorporated into the pharmaceutical compositions may be enantiomerically pure or enantiomerically enriched to such an extent that the effects of any dopaminergic activity associated with residual (6S)-2-amino-4,5,6,7-tetrahydro-6-(propylamino)benzothiazole (pramipexole) is either absent or sufficiently small to allow for high dosage administration of dexpramipexole relative to enantiomerically pure or enantiomerically enriched pramipexole. A description of methods for producing high purity dexpramipexole can be found in U.S. application Ser. No. 12/049,235, which is hereby incorporated by reference in its entirety. In some embodiments, treatment with dexpramipexole that may include administering daily doses of about 100 mg or more, about 125 mg or more, about 150 mg or more, 300 mg or more, 400 mg or more, 500 mg or more, or 600 mg or more without the adverse side effects associated with dopaminergic agonism. For example, daily doses of dexpramipexole of about 150 mg or more or about 300 mg or more may be administered without an apparent impact on heart rate, blood pressure, or other cardiac activity that can be measured using, for instance, ECG or blood pressure cuff that would otherwise be indicative of treatment with a dopamine agonist. In contrast, adverse side-effects associated with low dose pramipexole treatment (less than 5 mg per day) include, but are not limited to, dizziness, hallucination, nausea, hypotension, somnolence, constipation, headache, tremor, back pain, postural hypotension, hypertonia, depression, abdominal pain, anxiety, dyspepsia, flatulence, diarrhea, rash, ataxia, dry mouth, extrapyramidal syndrome, leg cramps, twitching, pharyngitis, sinusitis, sweating, rhinitis, urinary tract infection, vasodilatation, flu syndrome, increased saliva, tooth disease, dyspnea, increased cough, gait abnormalities, urinary frequency, vomiting, allergic reaction, hypertension, pruritis, hypokinesia, nervousness, dream abnormalities, chest pain, neck pain, paresthesia, tachycardia, vertigo, voice alteration, conjunctivitis, paralysis, tinnitus, lacrimation, mydriasis and diplopia. Administrations of about 100 mg or more, about 125 mg or more, about 150 mg or more, 300 mg or more, 400 mg or more, 500 mg or more, or 600 mg or more per day of dexpramipexole have not been shown cause any of these side-effects.

Moreover, because dexpramipexole is well tolerated, in some embodiments, treatment including administration of daily doses of about 100 mg or more, about 125 mg or more, about 150 mg or more, 300 mg or more, 400 mg or more, 500 mg or more, or 550 mg or more of dexpramipexole may be carried out for prolonged periods of time such as, for example, 12 weeks or more, 6 months or more, 1 year or more and, in certain embodiments, for 2, 3, 5 or 10 years or more, and in other embodiments, for an indefinite period of time of Accordingly, embodiments of the invention include methods of treating ALS may include administering dexpramipexole for an extended or prolonged period of time. In some embodiments, the extended period of time may be about 12 weeks or longer, about 6 months or longer, about 1 year or longer, and in other embodiments, a method of treating ALS comprises administering dexpramipexole on a maintenance dosing regimen. In such embodiments, the maintenance dosing regimen may include administering about 100 mg or more, about 125 mg or more, about 150 mg or more, 300 mg or more, 500 mg or more, or 550 mg or more of dexpramipexole per day without any titration (or an initial dosing regimen of less than the maintenance dose). Thus, various embodiments are directed to maintenance therapy in which a dosing schedule for dexpramipexole is maintained for an extended period of time without titration or otherwise changing the dosing schedule. In such embodiments, the extended period of time may be about 12 weeks or longer, about 6 months or longer, about 1 year or longer, 2, 3, 4, 5, or 10 years or longer, and in certain embodiments, an indefinite period of time. In other embodiments, the maintenance dosing may include administering less than the initial daily dose, such as, less than about 150 mg or less than about 300 mg of dexpramipexole per day. Additionally, without wishing to be bound by theory, the adverse effects associated with dopamine agonist treatment such as those described above may not develop after treatment with dexpramipexole has been carried out for a period of time of at least 12 weeks or more, and in some embodiments at least 6 months or 1, 2, 3, 5 or 10 years or more.

In further embodiments, an initial dosing regimen may be provided. In certain embodiments, the initial dosing regimen may include administering a higher dose of dexpramipexole than the maintenance dosing regimen as either a single administration or by administering an increased dosage for a limited period of time prior to beginning a maintenance dosing regimen. For example, in certain embodiments, the initial dosing regimen may be about 300 mg to about 500 mg or more of dexpramipexole per day, this initial dosing regimen may continue for 1, 2, 3, 4, 5, 6, or 7 days, up to 4 weeks, up to 8 weeks, or up to 12 weeks. Following the initial dosing regimen, the patient may be administered a maintenance dosing regimen of, for example, about 100 mg or more, about 125 mg or more, about 150 mg or more, 300 mg or more, 400 mg or more, 500 mg or more, or 550 mg or more of dexpramipexole for an indefinite period of time such as, for example, at least 12 weeks or more or at least 6 months or 1, 2, 3, 5 or 10 years or more. In some embodiments, patients undergoing a maintenance may be administered one or more higher dosage treatments at one or more times during the maintenance dosage regimen.

In various embodiments, dexpramipexole may be administered to any individual exhibiting the symptoms of a neurodegenerative disease or individuals predisposed to neurodegenerative disease. Non-limiting examples of neurodegenerative diseases that may be treated using dexpramipexole include Huntington's Chorea, metabolically induced neurological damage, Alzheimer's disease, senile dementia, age associated cognitive dysfunction, vascular dementia, multi-infarct dementia, Lewy body dementia, neurodegenerative dementia, neurodegenerative movement disorder, ataxia, Friedreich's ataxia, multiple sclerosis, spinal muscular atrophy, primary lateral sclerosis, seizure disorders, motor neuron disorder or disease, inflammatory demyelinating disorder, Parkinson's disease, amyotrophic lateral sclerosis (ALS), hepatic encephalopathy, and chronic encephalitis. Thus, the compositions and methods of the invention may be used to treat nearly any individual exhibiting symptoms of a neurological disease or susceptible to such diseases.

In particular embodiments, dexpramipexole may be used to treat ALS. For example, in some embodiments, individuals who were diagnosed with ALS within two years or less may be treated with dexpramipexole to reduce, eliminate or slow advancement of ALS or symptoms associated with ALS such as, for example, fine motor function loss, gross motor function, loss of bulbar function, and loss of respiratory function. In other embodiments, dexpramipexole may be administered to reduce or slow the advancement of symptoms including, but not limited to, trembling, loss of muscle control, loss of ability to write, low of ability to move or roll over, loss of speech, inability to swallow, difficulty breathing, and so on. In other embodiments, individuals with advanced symptoms or who were diagnosed with ALS more than 2 years before beginning treatment may be treated with dexpramipexole, and such individuals may respond to treatment by exhibiting a reduction or elimination of one or more ALS related symptoms or, in certain embodiments, the rate of symptom onset or advancement may be reduced, for example, the rate of motor function loss, loss of speech and/or swallowing may be slowed.

In further embodiments, a dose dependent response may be associated with treatment with dexpramipexole, and in certain embodiments, a dose dependent response may be enhanced when treatment is carried out for longer periods of time. For example, in some embodiments, a naïve patient who is administered a daily dose of for example, about 300 mg of dexpramipexole or more, about 500 mg or more, or about 600 mg or more may exhibit greater improvement in one or more symptoms of a neurological disease than a similarly situated naïve patient who is administered a daily dose of dexpramipexole less than 300 mg or less than 500 mg. In such embodiments, this improvement resulting from higher dosage administration may be apparent after a single treatment. However, in some embodiments, enhanced improvement in one or more symptoms as a result of administration of higher daily doses of dexpramipexole may be observed up to 6 months or more after beginning such treatment. Thus, in particular embodiments, treatment with higher doses of dexpramipexole may be carried out for prolonged periods of time, and the improvement associated with such dexpramipexole treatment may be realized after treatment has been carried out for a period of time of, for example, 1, 2, 3, 4, 5, 6, or 7 days, up to 1, 2, 4, 6, 8, 12, 24, or 48 weeks, up to 5, 10, 15, or 20 years, or any number of weeks between the recited values. In further embodiments, treatment with higher doses of dexpramipexole may be carried out as maintenance therapy, wherein the patient is administered such doses of dexpramipexole at the initiation of treatment and, thereinafter continue such doses of dexpramipexole over time. In each of the method embodiments described herein, any of the doses of dexpramipexole and/or any of the dosing regimens of dexpramipexole described herein may be used in such methods and continued administration of the such doses may be continued for any of the described periods of time.

In certain embodiments, the observed improvement in one or more symptoms may become enhanced as treatment progresses such that after an improvement is observed further improvements in the one or more symptoms may become evident with continued treatment. Without wishing to be bound by theory, a lag between beginning treatment and the first observation of improvement may be due to a period in which the dexpramipexole concentration in one or more of the patient's tissues increases to a threshold level where symptom improvement is observed. Any lag before observation of improvement may vary between patients and may vary depending on, for instance, the patient's demographics or characteristics such as, for example, age, progression of the disease, and/or the time between the onset of symptoms of the disease and beginning treatment.

In additional embodiments, dexpramipexole may be administered to patients in need of treatment for excessive weight loss associated with ALS. Without wishing to be bound by theory, the precipitous weight loss that is a cardinal symptom of ALS may be associated with increased energy expenditure, skeletal muscle hypermetabolism, and the systematic wasting of muscle tissue known as cachexia. In various embodiments, the total daily dose of dexpramipexole administered may be for example, less than 150 mg to 300 mg or greater, 400 mg or greater, 500 mg or greater, or 600 mg or greater. In each of the method embodiments described herein, any of the doses of dexpramipexole and/or any of the dosing regimens of dexpramipexole described herein may be used in such methods and continued administration of the such doses may be continued for any of the described periods of time.

In some embodiments, dexpramipexole may be administered by titration where one or more initial doses are less than 150 mg, less than 300 mg, less than 400 mg, less than 500 mg, less than 600 mg, and so on when administered to naïve patients. Generally, pramipexole treatment requires titration because pramipexole has a significant adverse impact on naïve patients, and titration over the course of weeks in which the dosage regimen is periodically increased to reach higher dosages purportedly limits these adverse effects. In various embodiments, of the invention, no titration of dexpramipexole is required. Thus, if an effective daily dose of dexpramipexole is, for example, 150 mg or 300 mg, the initial dose of dexpramipexole may be 150 mg or 300 mg of dexpramipexole, and each daily dose thereafter may be 150 mg or 300 mg. Accordingly, the daily dose may be considered a “stable daily dose.” For example, dexpramipexole treatment can be initiated at high levels without the need for titration. Therefore, a naïve patient who requires a greater than about 150 mg or about 300 mg or more, 400 mg or more, or about 500 mg or more, or about 600 mg or more dose of dexpramipexole for treatment may be administered about 100 mg or more, about 125 mg or more, about 150 mg or more, 300 mg or more, 400 mg or more, 500 mg or more, or 600 mg or more of dexpramipexole during the first treatment without the onset of adverse effects as would be expected if pramipexole was administered at its terminal level during an initial treatment. Accordingly, embodiments of the invention are directed to a method of treating a patient with ALS including administering an effective amount of dexpramipexole without titration. In certain embodiments, the effective amount may be about 100 mg or more, about 125 mg or more, about 150 mg or more, 300 mg or more, 400 mg or more, 500 mg or more, or 600 mg or more daily, and in some embodiments, the effect amount may be about 300 mg or more daily. In particular embodiments, the effective amount may be administered in separate equal doses twice daily. In certain embodiments, the effective amount may be administered twice daily or about every 12 hours. In each of the method embodiments described herein, any of the doses of dexpramipexole and/or any of the dosing regimens of dexpramipexole described herein may be used in such methods and continued administration of the such doses may be continued for any of the described periods of time.

Embodiments of the invention are also directed to a dosage regimen for administering dexpramipexole. For example, in some embodiments, the dosage regimen may include an initial dose dexpramipexole in one or more unit doses, then a plurality of daily doses having an equal amount of dexpramipexole as the initial dose in one or more unit doses. Such embodiments are not limited by the amount of the initial dose and daily doses. For example, in particular embodiments, the initial dose and each of the plurality of daily doses may be from about 50 mg to about 300 mg or about 400 mg, or about 500 mg or about 600 mg of dexpramipexole. In other embodiments, the initial dose and each of the plurality of daily doses may be from about 100 mg or more to about 300 mg or about 400 mg or about 500 mg or about 600 mg of dexpramipexole, and in still other embodiments, the initial dose and each of the plurality of daily doses may be about 300 mg or more about, about 400 mg or more, about 500 mg or more, or about 600 mg or more of dexpramipexole. In some embodiments, the one or more unit doses of the dosage regimen may be 1 to 5 unit doses, and in such embodiments, each of the one or more unit doses may be substantially equal. In other embodiments, each unit dose of the dosage regimen may be a solid unit dose. Each of the dosage regimen for dexprramipexole described herein may be used in any of the methods, and the dosing regiment may be carried out using any of the compositions described herein.

In particular embodiments, dexpramipexole may be administered to ALS patients, and in such embodiments, the improvements observed in ALS patients treated with dexpramipexole may be significantly better than conventional treatments such as, for example, riluzole. In some embodiments, the improvement may be signified by greater than 20% increase in ALS Functional Rating Scale, Revised (ALSFRS-R) score, when compared to baseline scores taken before treatment, and in other embodiments, this improvement may be manifested in a greater than 30% increase in ALSRFS-R score. In certain embodiments, the improvement in ALSFRS-R score may become apparent in less than 9 months, and in some embodiment, less than 6, 3, or 1 month. Riluzole, the only approved treatment for ALS, has not demonstrated any effect on ALSFRS-R score even after prolonged treatment. The majority of clinicians and clinical researchers believe that a therapy that results in a change of 20% or greater in slope of ALSFRS-R score is clinically meaningful. Therefore, the rate of improvement observed during dexpramipexole treatment is considerably and surprisingly better than that of other ALS treatments or no treatment based on ALSFRS-R score.

In various embodiments, dexpramipexole may be administered for the treatment of ALS without incurring adverse events associated with, for example, riluzole, the current standard of pharmacological intervention for ALS. For example, the overall rates of adverse events may be higher among patients receiving riluzole concomitant with dexpramipexole or in conjunction with placebo. Headaches, for example, were reported by four times as many patients receiving riluzole as those not receiving riluzole.

In some embodiments, dexpramipexole may be administered to improve the general health of individuals having a neurological disease, and in other embodiments, dexpramipexole may be administered to alleviate one or more specific symptoms. For example, in particular embodiments, dexpramipexole may be administered to ALS patients to improve symptoms associated with for example, fine motor, speech and swallowing or a combination thereof. Without wishing to be bound by theory, in such embodiments, improvements in fine motor and speech and swallowing related symptoms may become apparent in a shorter period of time following the initiation of dexpramipexole treatment than, for instance, improvements in large motor function and pulmonary related symptoms. Thus, while improvements in large motor function and pulmonary related symptoms may be observed after treatment with dexpramipexole, in some embodiments, dexpramipexole may be administered to alleviate fine motor and speech and swallowing related symptoms more immediately than other ALS symptoms. Therefore, in certain embodiments, ALS patients treated with dexpramipexole may have an increased time before a feeding tube must be employed because such patients may retain the ability to masticate and swallow food stuffs under their own power.

In other embodiments, dexpramipexole may be administered to slow the rate of decline of a patient exhibiting symptoms of a neurological disease and/or to reduce mortality in such patients. In such embodiments, populations of patients diagnosed with a neurological disease such as, for example, ALS, may exhibit an increased time to death, an increased survival rate, and/or a decreased frequency of death as a result of treatment with dexpramipexole. Moreover, even in patients who succumb to ALS or another neurological disease treated with dexpramipexole, dexpramipexole treatment may improve the quality of life for such patients up to death.

The foregoing methods may comprising administering dexpramipexole on a dosing regimen to achieve a dose dependent, steady state AUC₀₋₁₂ (h×ng/mL) ranging from 836±234 to 2803±1635 to 6004±2700 at daily doses of 50 mg, 150 mg, and 300 mg, respectively, when administered in two equal doses twice daily.

In further embodiments, dexpramipexole treatment may be carried out in combination with other forms of treatment. In some embodiments, such combination therapy may produce synergistic effects, such that the effect of dexpramipexole is augmented wherein one or more symptoms show a dramatic improvement over pre-treatment levels. For example, in certain embodiments, dexpramipexole treatment may be carried out in combination with (simultaneously or concurrently) with riluzole without adverse effects or reduced symptom relief. In other embodiments, dexpramipexole may be administrated in combination with (simultaneously or concurrently) with an additional form of treatment including, but not limited, those set forth in U.S. Provisional No. 61/113,680 filed Dec. 12, 2208 and U.S. Provisional No. 61/090,094 filed Aug. 19, 2009, each of which are hereby incorporated by reference in their entirety without producing adverse effects.

In some embodiments, the pharmaceutical composition of dexpramipexole may achieve the effects described above by eliciting a neuroprotective, anti-oxidative, anti-apoptotic, or other beneficial cellular effects without the side-effects associated with dopamine agonists commonly used to treat neurodegenerative diseases. Without wishing to be bound by theory, the ability to deliver clinically effective doses of dexpramipexole without dose limiting side effects may be made possible by: (i) the synthesis of dexpramipexole that is pure within limits of the detection; and (ii) dexpramipexole possesses a substantially lower affinity for dopamine receptors than its enantiomer, pramipexole. Further details regarding the molecular basis for dexpramipexole neuroprotective, anti-oxidative, anti-apoptotic, etc. activity including a comparison of the activity of dexpramipexole versus pramipexole can be found in U.S. application Ser. No. 11/957,157 which is hereby incorporated by reference in its entirety.

Various embodiments of the invention include methods for treating a neurodegenerative disease by administering a therapeutically effective amount of dexpramipexole such as, for example, about 100 mg or more, about 125 mg or more, about 150 mg, or more or about 300 mg or more. In accordance with such embodiments, dexpramipexole may be formulated as a pharmaceutical or therapeutic composition by combining with one or more pharmaceutically acceptable carriers. In some embodiments, such pharmaceutical or therapeutic compositions may be formulated in tablet or capsule form for use in oral administration routes. The compositions and amounts of non-active ingredients in such a formulation may depend on the amount of the active ingredient, and on the size and shape of the tablet or capsule. Such parameters may be readily appreciated and understood by one of skill in the art.

In various embodiments, the pharmaceutical compositions of the invention may have a chiral purity for dexpramipexole of at least 99.5%, at least 99.6%, at least 99.7%, at least 99.8%, at least 99.9%, at least 99.95%, or in some embodiments, at least 99.99%. In particular embodiments, the chiral purity for dexpramipexole may be about 100%. Such high chirally pure dexpramipexole, allows for therapeutic and pharmaceutical compositions that may have a wide individual and daily dose range. As such, the present invention provides a composition including only dexpramipexole in a pharmaceutically acceptable dosage, and in some embodiments, such pharmaceutical compositions may further include a pharmaceutically acceptable carrier, excipient and/or diluent.

In certain embodiments, the amount of pramipexole, (6S)-2-amino-4,5,6,7-tetrahydro-6-(propylamino)benzothiazole, remaining in the chirally pure dexpramipexole may be an amount not exceeding about 1.0 mg, and in some embodiments, the amount of pramipexole may be an amount not exceeding about 0.75 mg, about 0.5 mg, about 0.25 mg, or about 0.125 mg. In particular embodiments, the amount of pramipexole in chirally pure dexpramipexole may be less than about 0.125 mg. Therefore, the amount of pramipexole that may be administered in pharmaceutical compositions containing the chirally pure dexpramipexole of various embodiments may be less than 1.0 mg/day, less than 0.5 mg/day, and in certain embodiments, less than 0.125 mg/day. Without wishing to be bound by theory, the amount of pramipexole in chirally pure dexpramipexole may be a non-effective dose such that any pramipexole in such compositions does not elicit a noticeable effect on patients who are administered the pharmaceutical compositions of the invention. For example, a 300 mg/day dose of dexpramipexole administered to a patient as a single unit dose containing chiral purity dexpramipexole at least about 99.8% may contain a non-effective dose pramipexole less than 1.0 mg/day, a 300 mg/day dose of about 99.9% chirally pure dexpramipexole may include non-effective dose amount of pramipexole less than 0.5 mg/day, and a 300 mg/day dose of about 99.98% dexpramipexole may include non-effective dose pramipexole of less than 0.125 mg/day.

Chirally pure dexpramipexole may be prepared or converted to a pharmaceutically acceptable salt of dexpramipexole. For example, in some embodiments, dexpramipexole may be formulated as (6R)-2-amino-4,5,6,7-tetrahydro-6-(propylamino)benzothiazole dihydrochloride, which is a pharmaceutical salt and may improve solubility of dexpramipexole in water. The conversion of (6R)-2-amino-4,5,6,7-tetrahydro-6-(propylamino)benzothiazole to an acceptable salt by any method known in the art. For example, (6R)-2-amino-4,5,6,7-tetrahydro-6-(propylamino)benzothiazole dihydrochloride may be prepared by a one step method in which (6R)-2-amino-4,5,6,7-tetrahydro-6-(propylamino)benzothiazole or (6R)-2-amino-4,5,6,7-tetrahydro-6-(propylamino)benzothiazole salt is reacted with concentrated HCl in an organic solvent such as, an alcohol, at a reduced temperature of, for example, from about 0° C. to about 5° C. An organic solvent, such as methyl tert-butyl ether, may then be added, and the reaction may be stirred for about one hour. The (6R)-2-amino-4,5,6,7-tetrahydro-6-(propylamino)benzothiazole dihydrochloride produced may be recovered from the reaction mixture by filtering, washing with an alcohol and vacuum drying.

The amount of dexpramipexole in such pharmaceutical composition oral suitable for oral administration may vary. For example, in some embodiments, the amount of dexpramipexole in such compositions may be from about 25 mg to about 1000 mg, about 50 mg to about 1000 mg, from about 100 mg to about 1000 mg, from about 125 mg to about 1000 mg, from about 150 mg to about 1000 mg, from about 300 mg to about 1000 mg, from about 500 mg to about 1000 mg, from about 600 to about 1000 mg, and in certain embodiments, the amount of dexpramipexole may be from about 60 mg to about 300 mg. Each of the compositions embodied herein, may be used in any of the methods or dosage regimen described herein.

In various embodiments, the daily dose of dexpramipexole may be administered as a single daily dose or may be divided into two or more doses of equal or unequal amount administered throughout the day. For example, in some embodiments, about 100 mg or more, about 125 mg or more, about 150 mg or more, 300 mg or more, 500 mg or more, or 600 mg or more of dexpramipexole may be administered in 1 to 5 doses each containing an equal amount of dexpramipexole, and in other embodiments, about 100 mg or more, about 125 mg or more, about 150 mg or more, 300 mg or more, 500 mg or more, or 600 mg or more of dexpramipexole may be administered in 2 or 3 doses throughout the day. In still other embodiments, about 100 mg or more, about 125 mg or more, about 150 mg or more, 300 mg or more, 500 mg or more, or 600 mg or more of dexpramipexole may be administered in 2 or 3 doses wherein the one dose contains a higher concentration of dexpramipexole. For example, one dose of a 300 mg regimen may contain 100 mg of dexpramipexole and a second dose administered at a different time during the day may contain 200 mg of dexpramipexole. The daily doses may be used in any of the methods or dosage regimen described herein.

The pharmaceutical or therapeutic compositions of the invention may be prepared, packaged, sold in bulk, as a single unit dose, or as multiple unit doses and can be administered in the conventional manner by any route where they are active. For example, the compositions may be administered orally, ophthalmically, intravenously, intramuscularly, intra-arterially, intramedularry, intrathecally, intraventricularly, transdermally, subcutaneously, intraperitoneally, intravesicularly, intranasally, enterally, topically, sublingually, rectally by inhalation, by depot injections, or by implants or by use of vaginal creams, suppositories, pessaries, vaginal rings, rectal suppositories, intrauterine devices, and transdermal forms such as patches and creams. Specific modes of administration will depend on the indication. The selection of the specific route of administration and the dose regimen may be adjusted or titrated by the clinician according to known methods in order to obtain the optimal clinical response. All of the methods described herein may be carried out by administering dexparmipexole by any such route for administration described herein. Additionally, dexpramipexole may be delivered by using any such rout of administration for all of the dosage regimen described herein.

Pharmaceutical formulations containing dexpramipexole in a solid dosage may include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder; comprising an effective amount of a polymer or copolymer of the present invention. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted.

For oral administration, the compounds can be formulated readily by combining these compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

In some embodiments, pharmaceutical compositions may be suitable for oral administration such as, for example, a solid oral dosage form or a capsule, and in certain embodiments, the composition may be a tablet. Such tablets may include any number of additionally agents such as, for example, one or more binder, one or more lubricant, one or more diluent, one or more lubricant, one or more surface active agent, one or more dispersing agent, one or more colorant, and the like. Such tablets may be prepared by any method known in the art, for example, by compression or molding. Compressed tablets may be prepared by compressing in a suitable machine the ingredients of the composition in a free-flowing form such as a powder or granules, and molded tablets may be made by molding in a suitable machine a mixture of the powdered compound moistened with an inert liquid diluent. The tablets, of some embodiments, may be uncoated and, in other embodiments, they may be coated by known techniques.

In other embodiments prepared for oral administration, the pharmaceutical compositions of the invention may be provided in a dragee cores with suitable coatings. In such embodiments, dragee cores may be prepared suing concentrated sugar solutions, which may optionally contain gum arabic, talc, polyvinyl pyrrolidone, carbopol gel, polyethylene glycol, and/or titanium dioxide, lacquer solutions, and suitable organic solvents or solvent mixtures. In some embodiments, dyestuffs or pigments may be added to the tablets or dragee coatings for identification or to characterize different combinations of active compound doses. In yet other embodiments, pharmaceutical compositions including an effective amount of dexpramipexole prepared for oral administration may include, but are not limited to, push-fit capsules made of gelatin, as well as soft, sealed capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

In embodiments in which the tablets and dragee cores are coated, the coatings may delay disintegration and absorption in the gastrointestinal tract and thereby providing a sustained action over a longer period. Additionally, such coatings may be adapted for release dexpramipexole in a predetermined pattern (e.g., in order to achieve a controlled release formulation) or it may be adapted not to release the active compound until after passage of the stomach (enteric coating). Suitable coatings encompassed by such embodiments may include, but are not limited to, sugar coating, film coating (e.g., hydroxypropyl methylcellulose, methyl-cellulose, methyl hydroxyethylcellulose, hydroxypropylcellulose, carboxymethylcellulose, acrylate copolymers, polyethylene glycols and/or polyvinylpyrrolidone), or an enteric coating (e.g., methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material such as, for example, glyceryl monostearate or glyceryl distearate may be incorporated into the coatings of some embodiments. In still other embodiments, solid tablet compositions may include a coating adapted to protect the composition from unwanted chemical changes, for example, to reduce chemical degradation prior to the release of the active drug substance.

Pharmaceutical composition suitable for oral administration encompassed in embodiments of the invention may include a therapeutically effective amount of dexpramipexole and a non-effective dose amount of pramipexole and may further include one or more diluent, one or more disintegrant, one or more lubricant, one or more pigment or colorant, one or more gelatin, one or more plasticizer and the like. For example, in some embodiments, a tablet may include dexpramipexole, from about 20% to about 50% by weight of diluent in an amount, from about 10% to about 30% by weight of a second diluent, from about 2% to about 6% by weight of a disintegrant, and from about 0.01% to about 2% by weight of a lubricant, and in particular embodiments, such tablets may include an effective amount of dexpramipexole, from about 20% to about 50% by weight microcrystalline cellulose, about 10% to about 30% by weight, from about 2% to about 6% crospovidone or croscarmellose, and from about 0.01% to about 2% by weight magnesium stearate. In further embodiments, the pharmaceutical composition may include any amount or combination of microcrystalline cellulose, mannitol, sodium, crospovidone, croscarmellose magnesium stearate, or combination thereof.

In such embodiments, the pharmaceutical composition suitable for oral administration may include at least about 50 mg of dexpramipexole, and in some embodiments, such pharmaceutical compositions may include at least about 75 mg of dexpramipexole, at least about 100 mg of dexpramipexole, at least about 150 mg of dexpramipexole, at least about 200 mg of dexpramipexole, at least about 250 mg of dexpramipexole, 300 mg of dexpramipexole, at least about 500 mg of dexpramipexole, at least about 600 mg of dexpramipexole, at least about 750 mg of dexpramipexole, or at least about 1000 mg of dexpramipexole. In certain embodiments, such pharmaceutical compositions suitable for oral administration prepared at any doseage described above may include a non-effective dose amount of pramipexole of less than about 0.125 mg.

In some embodiments, the pharmaceutical compositions including dexpramipexole may be prepared as suspensions, solutions or emulsions in oily or aqueous vehicles suitable for injection. In such embodiments, such liquid formulations may further include formulatory agents such as suspending, stabilizing and/or dispersing agents formulated for parenteral administration. Such injectable formulations may be administered by any route, for example, subcutaneous, intravenous, intramuscular, intra-arterial or bolus injection or continuous infusion, and in embodiments in which injectable formulations are administered by continuous infusion, such infusion may be carried out for a period of about 15 minutes to about 24 hours. In certain embodiments, formulations for injection can be presented in unit dosage form, e.g., in ampoules or in multi-dose containers, with an added preservative.

In other embodiments, dexpramipexole may be formulated as a depot preparation, and such long acting formulations can be administered by implantation (for example subcutaneously or intramuscularly) or by intramuscular injection. Depot injections can be administered at about 1 to about 6 months or longer intervals. Thus, for example, the compounds can be formulated with suitable polymeric or hydrophobic materials (for example, as an emulsion in an acceptable oil) or ion exchange resins, or as sparingly soluble derivatives, for example, as a sparingly soluble salt.

In still other embodiments, pharmaceutical compositions including dexpramipexole may be formulated for buccal or sublingual administration. In such embodiments, the pharmaceutical compositions may be prepared as chewable tablets, flash melts or lozenges formulated in any conventional manner.

In yet other embodiments, pharmaceutical compositions including dexpramipexole may be formulated for administration by inhalation. In such embodiments, pharmaceutical compositions according to the invention may be delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

In further embodiments, pharmaceutical compositions including dexpramipexole can be formulated in rectal compositions such as suppositories or retention enemas, e.g., containing conventional suppository bases such as cocoa butter or other glycerides.

In some embodiments, pharmaceutical compositions including dexpramipexole may be formulated for transdermal administration. For example, such pharmaceutical compositions may be prepared to be applied to a plaster or applied by transdermal, therapeutic systems that are supplied to the patient. In other embodiments, pharmaceutical and therapeutic compositions including dexpramipexole for transdermal administration may include a suitable solid or gel phase carriers or excipients such as, but are not limited to, calcium carbonate, calcium phosphate, various sugars, starches, cellulose derivatives, gelatin, and polymers such as, e.g., polyethylene glycols.

In some embodiments, pharmaceutical compositions including dexpramipexole may be administered alone as a single therapeutic agent. In other embodiments, the pharmaceutical compositions including dexpramipexole may be administered in combination with one or more other active ingredients, such as, for example, adjuvants, protease inhibitors, or other compatible drugs or compounds where such combination is seen to be desirable or advantageous in achieving the desired effects of the methods described herein.

The embodiments for disease states, patient type (naïve vs. not naïve), daily dose amounts, no observable adverse effect level dose amounts, non-effective dose amounts, and chiral purities for the methods of the invention, which are described herein separately for the sake of brevity, can be joined in any suitable combination.

### EXAMPLES

### Example 1

Example 1 was a randomized, placebo-controlled, double-blind, parallel-group, multi-center study to evaluate the safety, tolerability, and clinical effects of oral administration of 3 dosage levels of dexpramipexole vs. placebo for 12 weeks in patients with ALS. In Part 1, 80 eligible patients were to be randomized to 1 of 4 treatment groups in a 1:1:1:1 ratio for 12 weeks of treatment with dexpramipexole (50 mg, 150 mg, or 300 mg total daily dose) or placebo. Doses were administered as 25 mg, 75 mg, or 150 mg dexpramipexole every twelve hours, or placebo every twelve hours.

Safety evaluations were performed at study visits scheduled at Baseline, Day 1 Post-Dose, Week 1, Week 2, Week 4, Week 8, and Week 12 (or end-of-study if a subject discontinued prematurely). Clinical status assessments, including the ALS Functional Rating Scale (revised) (ALSFRS-R), vital capacity (VC), and McGill Quality-of-Life Single-Item Scale (McGill SIS), were performed at Baseline, Week 4, Week 8, and Week 12 (or end-of-study if a subject discontinued prematurely). CSF and plasma samples were collected at Baseline and Week 12 for proteomic analysis to study potential surrogate markers indicative of disease progression in ALS and to evaluate changes in surrogate markers that may be associated with dexpramipexole treatment.

Eighty (80) patients were planned to be enrolled and randomized to 1 of 4 treatment groups in a 1:1:1:1 ratio for a distribution of 20 subjects per treatment arm. Subjects aged 21 to 80 years with a clinical diagnosis of familial or sporadic ALS who met the possible, laboratory-supported probable, probable, or definite criteria for a diagnosis of ALS according to the World Federation of Neurology El Escorial criteria; who are <24 months from ALS symptom onset; and who had an upright VC>65% of predicted for age, height, and gender were eligible to enroll. Subjects using riluzole (Rilutek®) at the time of randomization were required to continue taking riluzole at the same dosage level throughout the study. Women of childbearing potential (WOCBP) must have agreed to use 2 methods of interventional contraception throughout participation in the study. Surgical sterilization (i.e., vasectomy) of the male partner was considered as one effective method of interventional contraception. However, using the rhythm method was not considered sufficient. WOCBP must have also agreed to pregnancy testing and have had a negative pregnancy test at periodic study visits. Non-surgically sterilized men whose sexual partners were WOCBP must have agreed to ensure their partners used at least one highly effective contraception method (e.g., oral, injected or implanted hormonal methods, or intrauterine device) prior to study entry, for the duration of the study, and for 28 days after the last dose of study medication.

Clinical status was assessed by administration of (1) the ALSFRS-R to assess functional status; (2) VC to assess pulmonary function; and (3) the McGill single-item scale (SIS) to assess general quality-of-life. Plasma and CSF samples were collected to assess potential drug-related changes in potential surrogate markers of motor neuron stress and damage, such as levels of cystatin C. Sample analyses were not completed for the Part 1 study synopsis, but these data will be reported in the Part 2 study report.

A total of 102 subjects were randomized at 20 US sites and received at least 1 dose of study medication: 27 subjects received placebo, 23 subjects received dexpramipexole 50 mg, 26 subjects received dexpramipexole 150 mg, and 26 subjects received dexpramipexole 300 mg. Enrollment by site ranged from 1 to 10 subjects. A total of 98 subjects (96%) completed Part 1 of the study. Two subjects (1 in the 50 mg dose group and 1 in the 300 mg dose group) withdrew consent and 2 subjects (1 in the placebo group and 1 in the 300 mg group) discontinued due to an adverse event. The mean duration of disease at time of randomization (mean time from ALS symptom onset to Day 1 of dosing in the study) across treatment groups was 427 days (15.25 months). The placebo and 150 mg groups had the longest mean durations of disease (473 and 458 days, respectively), while the 50 mg and 300 mg groups had the shortest mean durations of disease (381 and 391 days, respectively).

No deaths occurred during Part 1 of the study. A total of 6 SAEs were reported by 5 subjects: 2 subjects in the 50 mg group and 3 subjects in the 300 mg group. None of the SAEs were judged by the investigator to be related to treatment with study medication. Two subjects discontinued the study due to an adverse event: one (1) due to agitated depression (placebo) and one (1) due to nausea (300 mg). Ninety-two out of 102 subjects (90%) reported at least 1 AE, and the percentage of subjects reporting AEs was similar across the treatment groups (93%, 83%, 96%, and 89% for the placebo, 50 mg, 150 mg, and 300 mg groups, respectively). AEs reported by at least 10% of subjects in decreasing frequency of the total number of subjects reporting an event in the combined active treatment groups were fall (32%), muscular weakness (24%), post lumber puncture syndrome (19%), headache (13%), and nausea (11%). Adverse events reported by at least 5% of subjects in the combined active treatment groups at an incidence≧5% greater than placebo included fall, nausea, and arthralgia. The percentage of subjects reporting at least 1 AE that had been judged by the investigator to be possibly or probably treatment-related was 22% (placebo), 17% (50 mg), 42% (150 mg), and 27% (300 mg).

There was no difference across the treatment groups in the incidence of ECG abnormalities that met pre-specified criteria for potential clinical significance. None of the subjects had abnormalities in hematology parameters that met pre-specified criteria for potential clinical significance. The number of adverse events reported during the 12 week study are summarized in Table 1. These data indicate that dexpramipexole is safe and well-tolerated.

TABLE 1
Adverse Events

| | | Total AE's |
|---|---|---|
| Group | N | N (%) |
| Placebo | 27 | 25 (92.6%) |
| 50 mg | 23 | 19 (82.6%) |
| 150 mg | 26 | 25 (96.2%) |
| 300 mg | 26 | 23 (88.5%) |

ALSFRS-R mean scores at baseline were similar across the treatment groups. The mean changes from baseline to endpoint in ALSFRS-R total scores were −3.6 (placebo), −5.0 (50 mg), −3.3 (150 mg), and −2.2 (300 mg). The median changes from baseline to endpoint in ALSFRS-R scores were −4.0 (placebo), −3.0 (50 mg), −2.5 (150 mg), and −2.0 (300 mg). In the 300 mg group, the mean and median decline from baseline to study endpoint in the ALSFRS-R score was reduced by 39% and 50%, respectively, compared with the placebo group.

The primary analysis of ALSFRS-R data specified in the SAP was a linear mixed-effects analysis of the treatment effect on the slope of ALSFRS-R scores during the study. The slope observed for the placebo group was −1.278, whereas the slope observed for the 300 mg group was −0.878, a 31% improvement relative to the placebo group. The primary analysis of treatment effects on the slope of ALSFRS-R scores across treatment groups was p=0.1087.

An exploratory analysis of the apparent positive trend in dose-response was conducted by regression of change from baseline in ALSFRS-R total scores at study endpoint on dose. This analysis was not significant (p=0.0655). When selected covariates (gender, duration of ALS symptoms at baseline, concomitant riluzole use, and baseline ALSFRS-R score) were added to the regression model, the ANCOVA was significant (p=0.0475). For both analyses the lower mean change in the 50 mg group in ALSFRS-R at endpoint than placebo contributed to the significance of these tests.

ANCOVA of change from baseline in ALSFRS-R total scores on dose was conducted to adjust for selected baseline covariates (gender, duration of ALS symptoms at baseline, concomitant riluzole use, and baseline ALSFRS-R score). The change from baseline in ALSFRS-R total scores at study endpoint (LOCF) was improved in the 300 mg group compared to the placebo group (p=0.0412).

There was no effect of riluzole on the change from baseline to study endpoint in ALSFRS-R scores across all treatment groups. In the placebo group, 16 subjects received riluzole and 11 subjects did not. For this group, the mean change from baseline to study endpoint in ALSFRS-R scores were −3.6 (placebo with riluzole) and −3.5 (placebo without riluzole), respectively.

The mean score on this 10-point McGill Quality of Life (QOL) scale at baseline was 7.0 (placebo), 6.8 (50 mg), 7.3 (150 mg), and 8.1 (300 mg). Median scores at baseline were 7.0 (placebo and 50 mg) and 8.0 (150 mg and 300 mg). The mean change from baseline in QOL scores at endpoint were 0.0 (placebo), −0.6 (50 mg), −0.6 (150 mg), and −0.9 (300 mg). The mean change from baseline at each time point in the placebo group was influenced by one outlier who reported a score of 0 at baseline (due to discomfort associated with the lumber puncture procedure) and subsequently reported a score of 10 for all on-treatment visits.

Pharmacokinetic analyses were based on data from 20 subjects in the 25 mg Q12H (n=8), 75 mg Q12H (n=8), and 150 mg Q12H (n=4) groups. Pharmacokinetics were linear across this range of doses. Steady-state was achieved before study Day 10, the earliest PK study day, consistent with the observed elimination half-life of 6.63 hours to 8.73 hours. CL/F and Vz/F were similar across dose groups. Likewise, Tmax was similar across treatment groups at 1.77, 1.82, and 1.70 hours for the 25, 75, and 150 mg every twelve hour groups, respectively. Cₘₐₓ and AUC increased proportionately with dose. The parameter estimates for total plasma clearance uncorrected for bioavailability (CL/F), volume of distribution uncorrected for bioavailability (Vz/F), and half-life (VA) are comparable between the 2 populations.

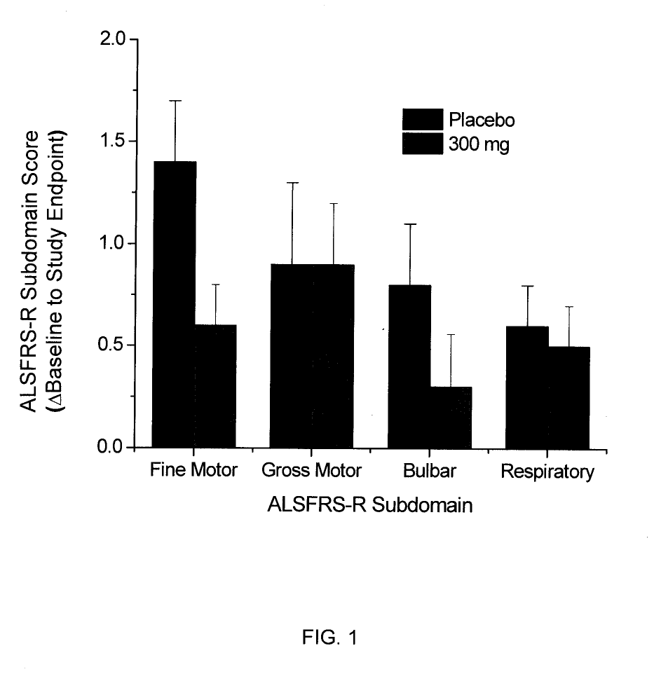

The ALSFRS-R is divided into 4 equal sections or subdomains, representing the effects of disease on fine motor function, gross motor function, bulbar function, and respiratory function. These subdomains decline at different rates, in the order listed (highest rate to lowest). Among subjects receiving placebo in Part 1, the fine motor subdomain score declined at a higher rate than the gross motor, bulbar, or respiratory subdomains (mean±SEM/% total score; −1.4±0.30/38%, −0.9±0.36/24%, −0.8±0.25/22%, −0.6±0.22/16%, respectively). The greatest difference at study endpoint between subjects receiving placebo and those receiving 300 mg/day dexpramipexole was in the mean fine motor domain (−1.4±0.30 vs. −0.6±0.24, p=0.043; FIG. 1).

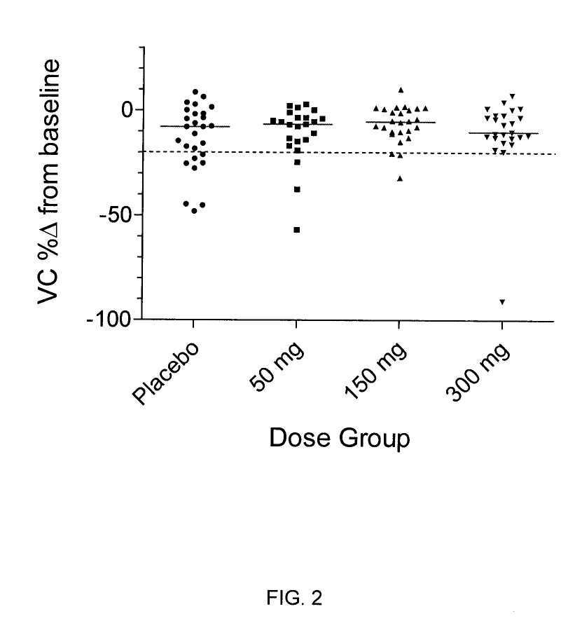

A 6-point or greater drop in ALSFRS-R total score from baseline has been used to identify subjects that failed to respond to drug treatment. In this trial, when a 6-point or greater drop in ALSFRS-R total score from baseline to 12 weeks in Part 1 was used to define treatment failure in a post hoc analysis, a significant dose-dependent effect was observed. The number of failures totaled 9 subjects (33%) in the placebo group; 8 subjects (35%) in the 50 mg/day group, 4 subjects (15%) in the 150 mg/day group, and 2 subjects (8%) in the 300 mg/day group (logistic regression analysis, p=0.014; FIG. 2). In FIG. 2, the failure line is defined as anything at or below the dotted line, and the red lines are the median decline at the indicated week.

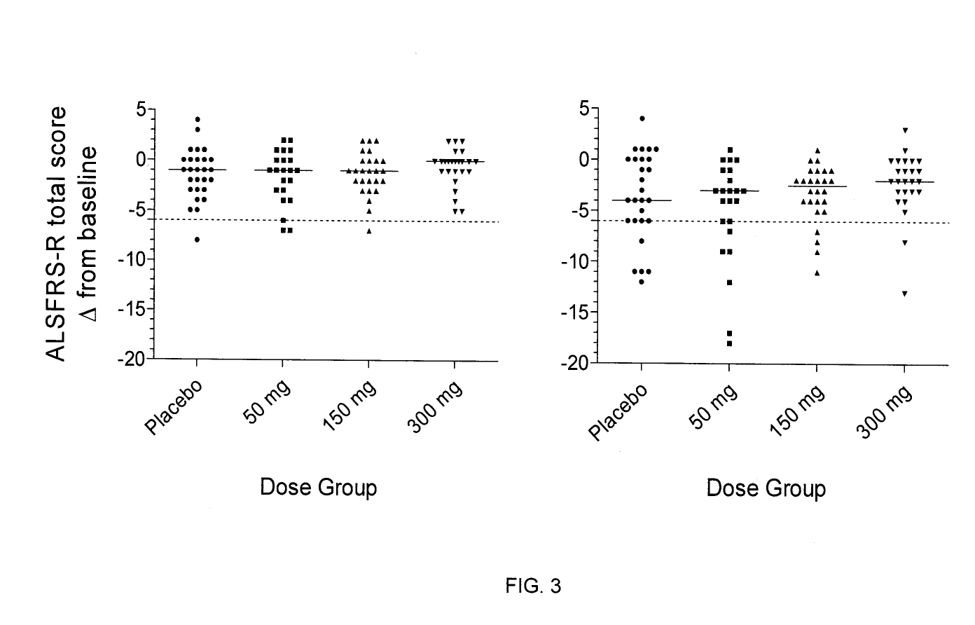

At baseline in Part 1, upright vital capacity (VC) values were similar in the four treatment groups (Table 2A, 2B). Based on a linear mixed-effects model, the slope of upright VC did not differ significantly across treatment groups (p=0.5438). However, the number of treatment failures, defined as a reduction in VC of 20% or greater from baseline to Week 12, totaled 8 subjects (30%) in the placebo group, 3 subjects (13%) in the 50 mg group, 3 subjects (12%) in the 150 mg group, and 1 subject (4%) in the 300 mg group (logistic regression analysis; p=0.028; FIG. 3). In FIG. 3, the red lines represent median decline in VC over the 12 weeks of Part 1, and the dotted line is the 20% change from baseline that is defined as the treatment failure level.

TABLE 2A
Unadjusted Slope Estimates

| | 50 mg | 300 mg | % reduction | p value |
|---|---|---|---|---|
| Fine | −0.311 | −0.211 | 32.15% | 0.1539 |
| Gross | −0.305 | −0.320 | −4.92% | 0.8187 |
| Bulbar | −0.364 | −0.310 | 14.84% | 0.5487 |
| Resp | −0.270 | −0.186 | 31.11% | 0.3491 |

TABLE 2B
Zero Imputation Slope
Estimates

| | 50 mg | 300 mg | % reduction | p value |
|---|---|---|---|---|
| Fine | −0.440 | −0.238 | 45.91% | 0.0189 |
| Gross | −0.390 | −0.338 | 13.33% | 0.4830 |
| Bulbar | −0.578 | −0.376 | 34.95% | 0.1289 |
| Resp | −0.575 | −0.255 | 55.65% | 0.0363 |

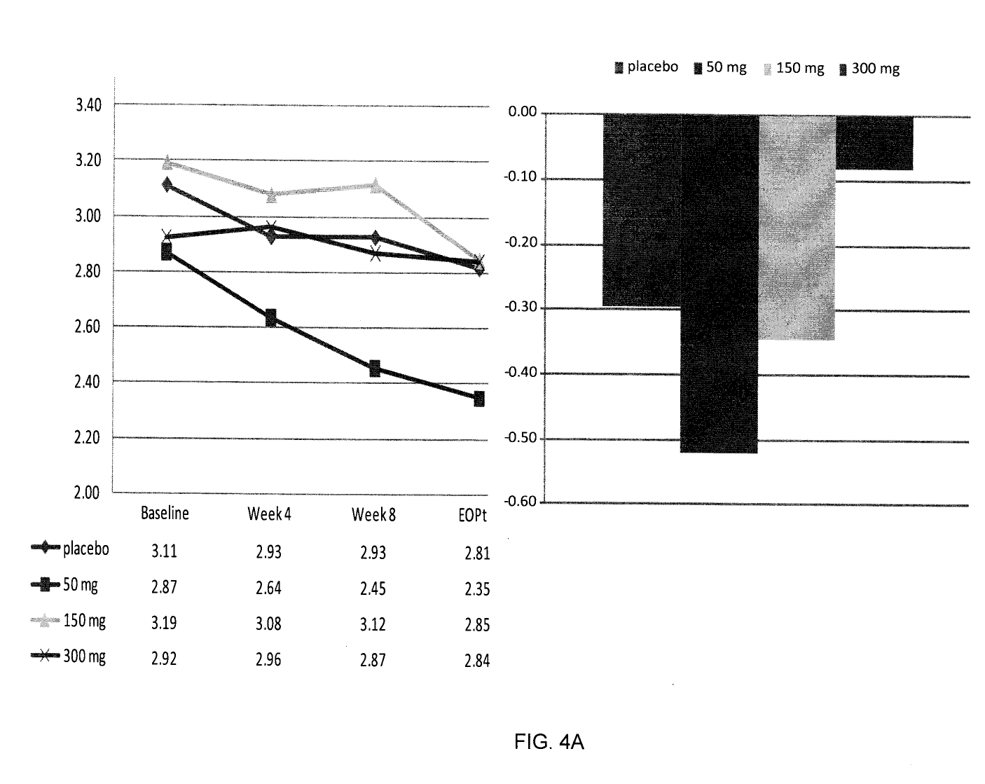

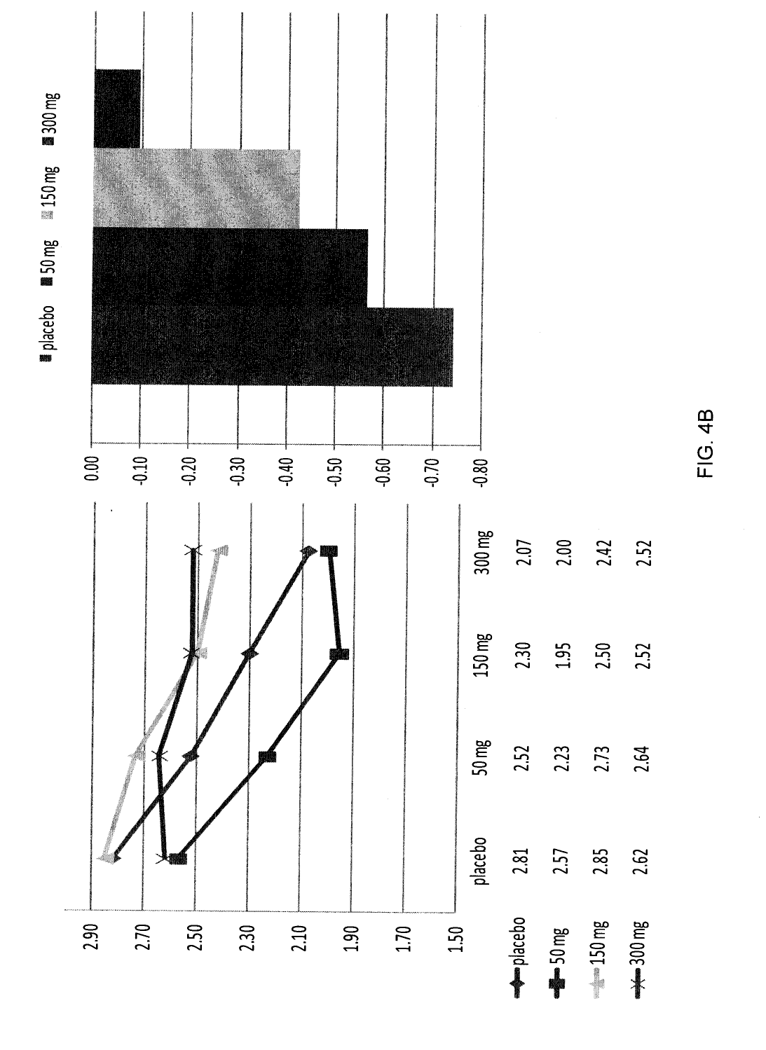

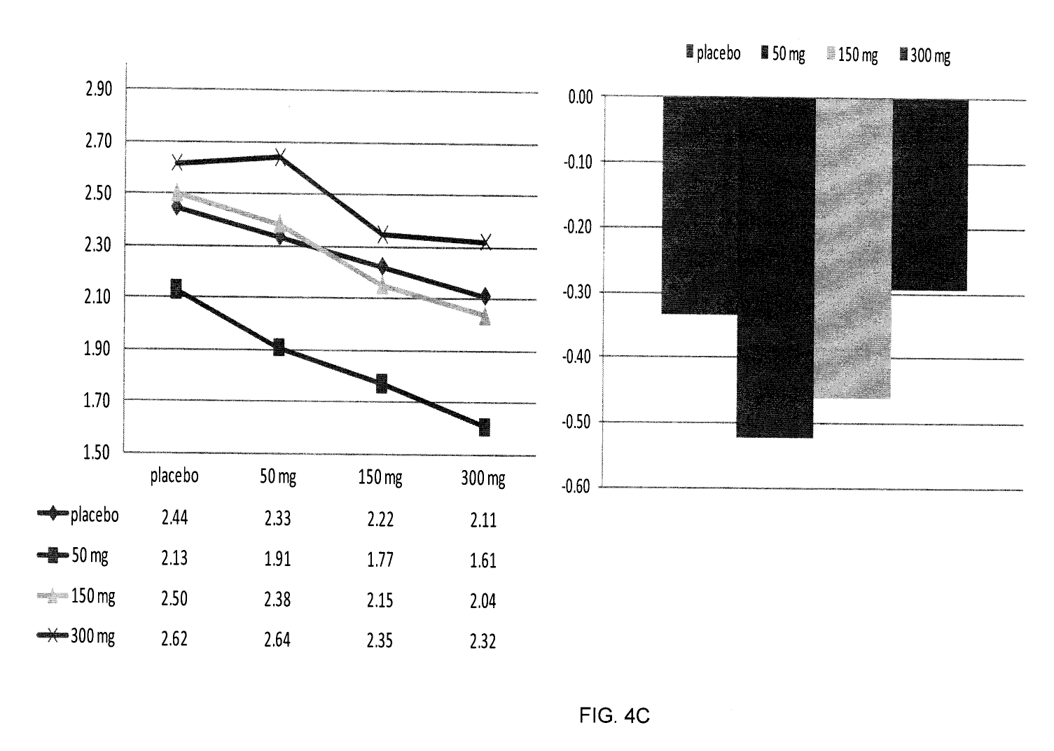

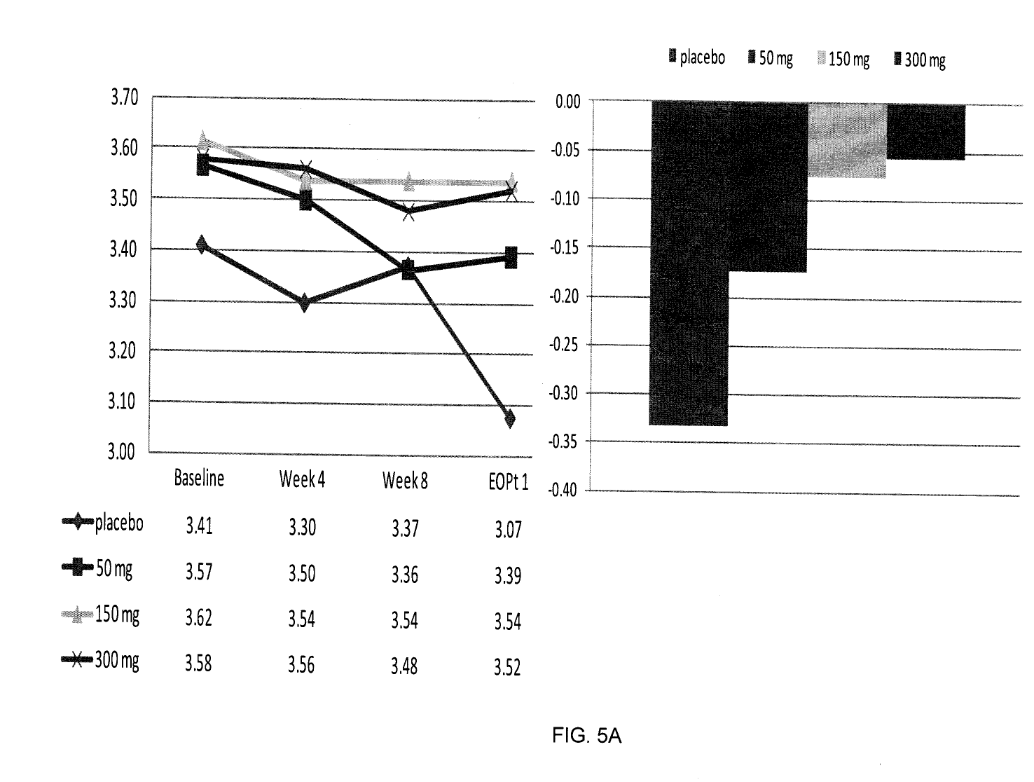

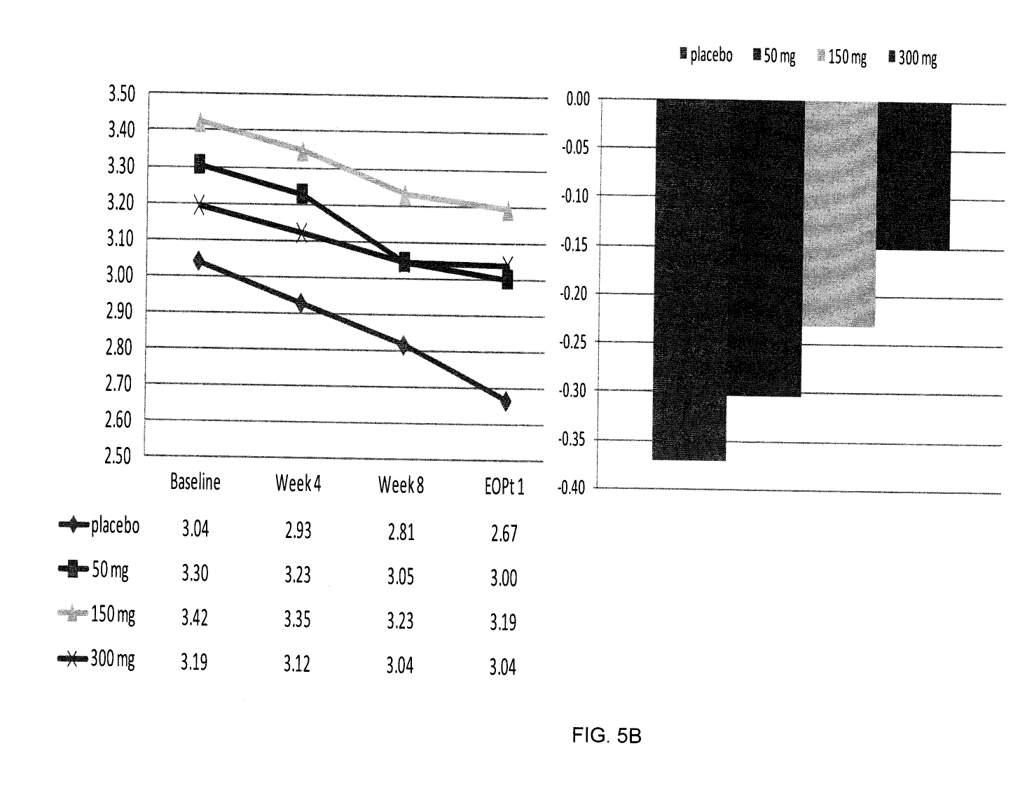

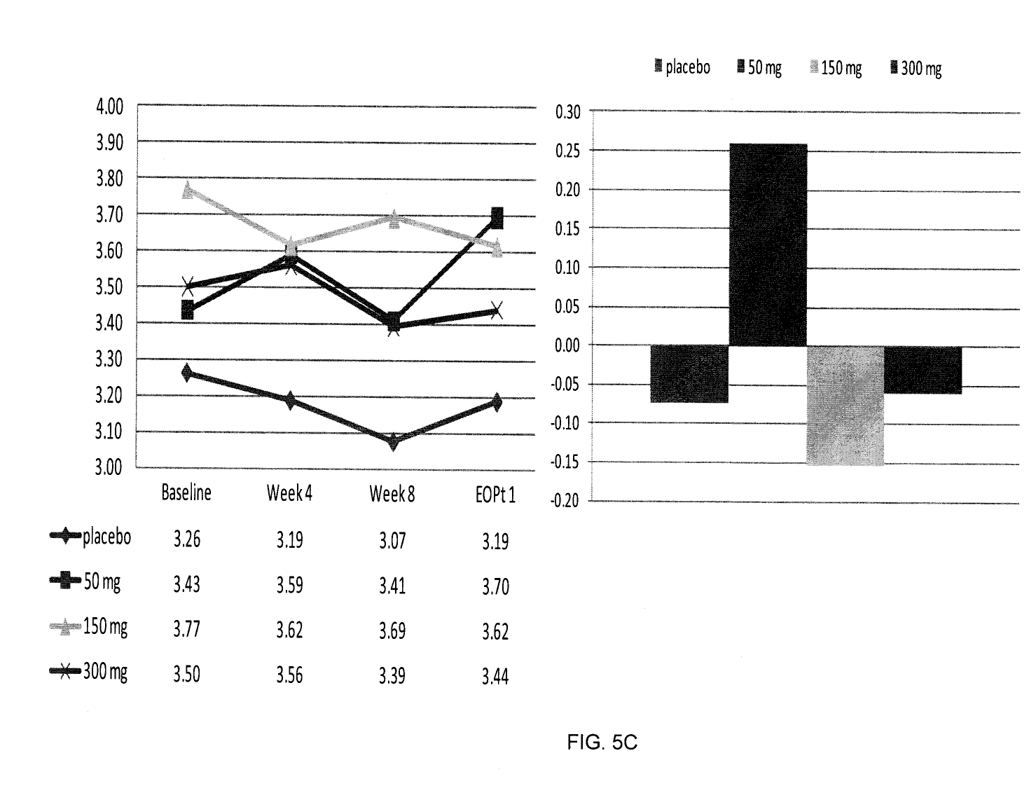

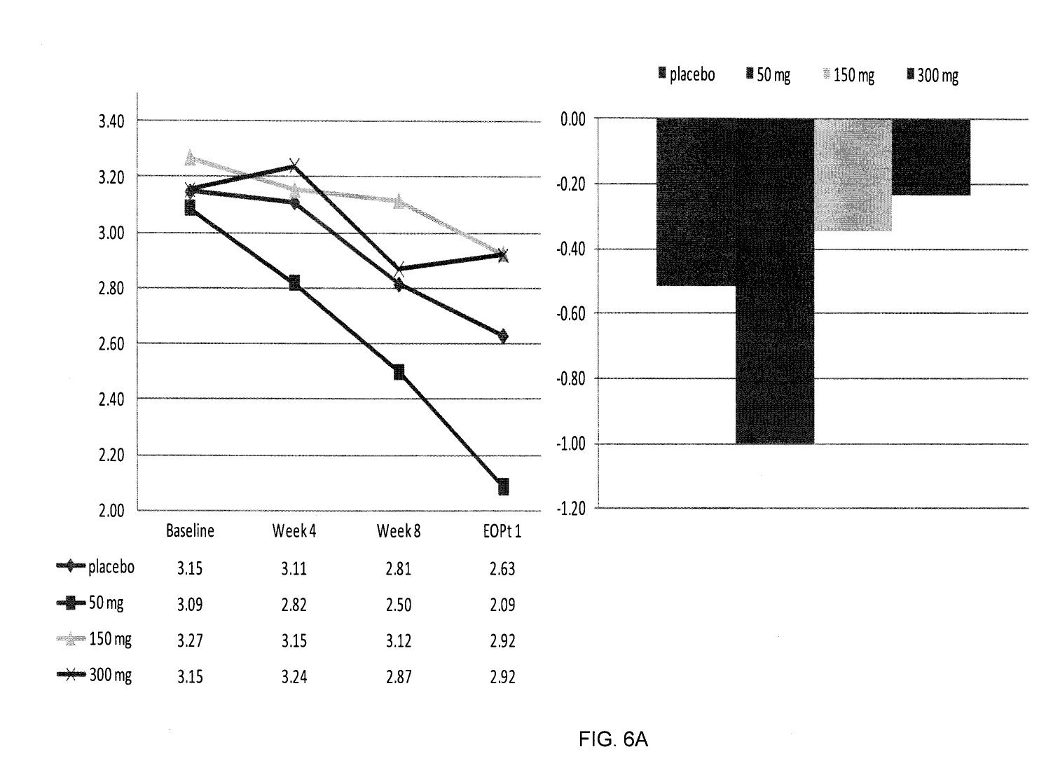

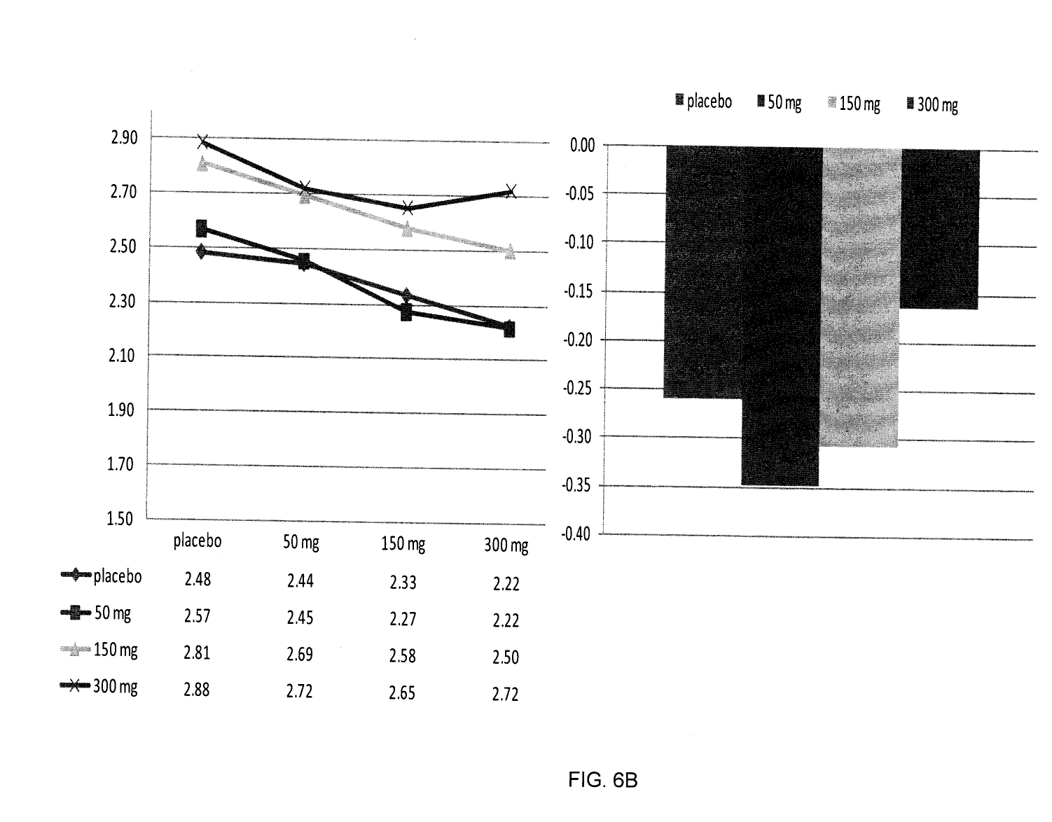

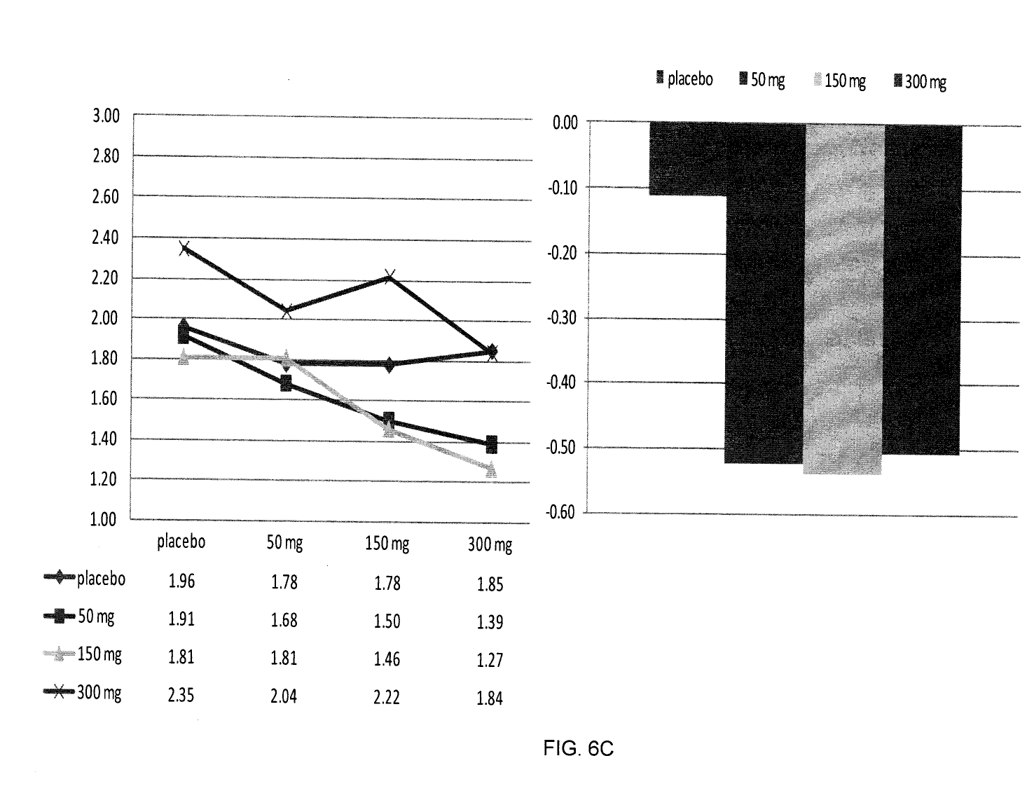

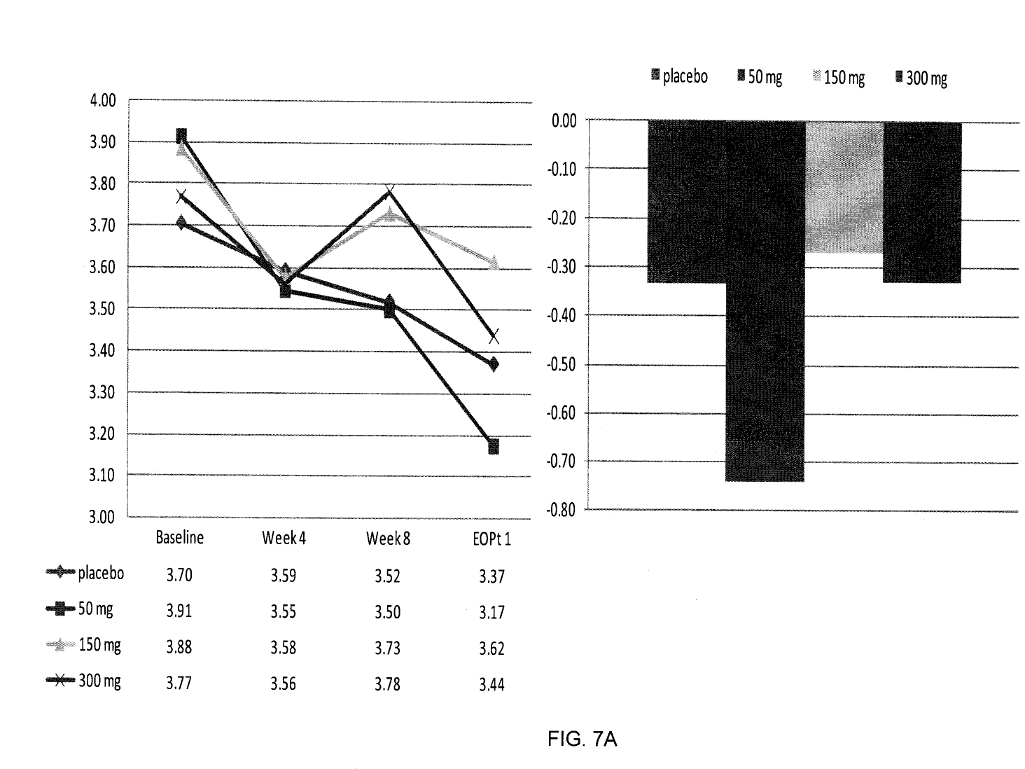

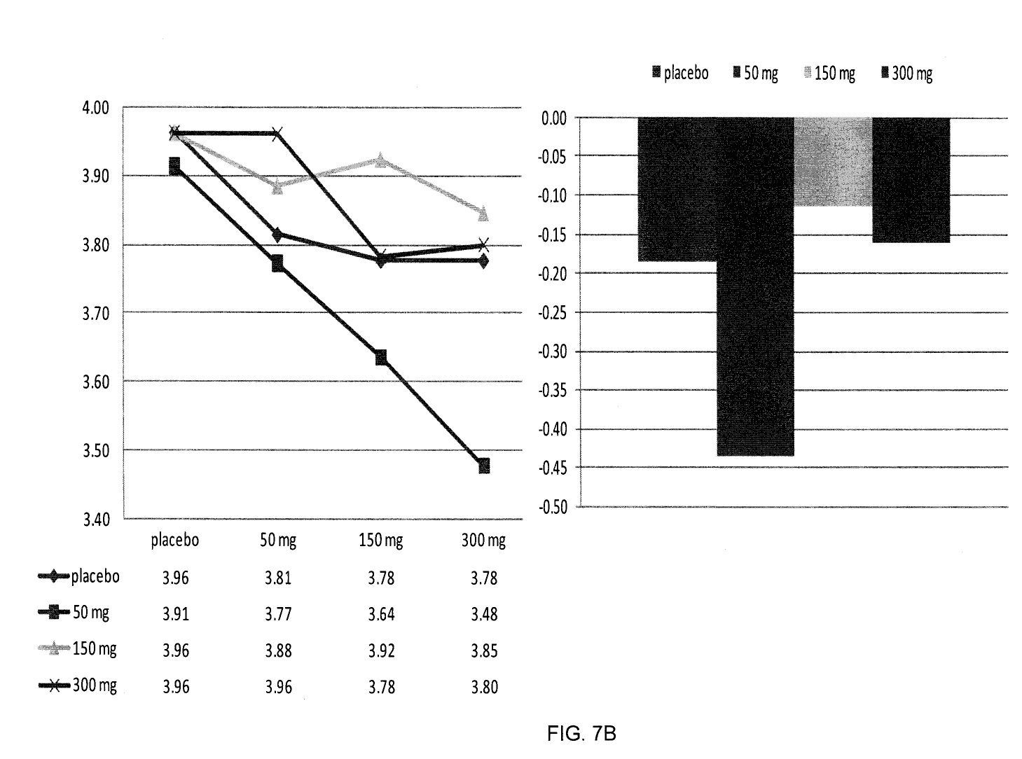

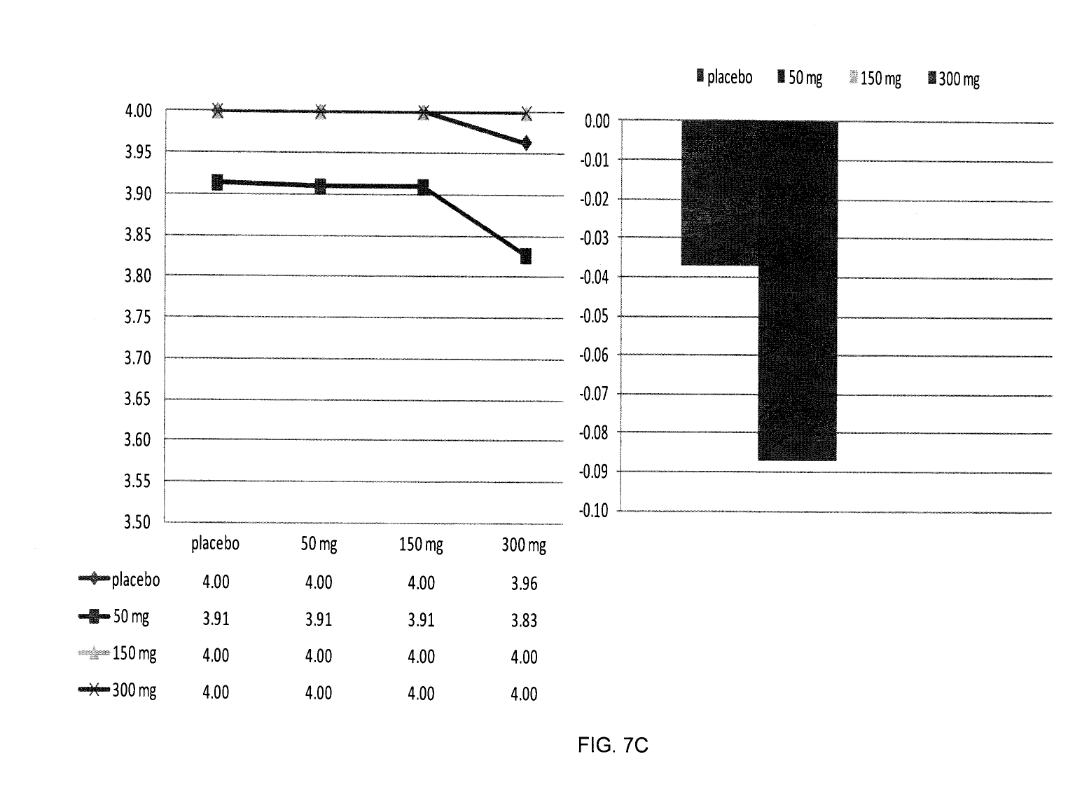

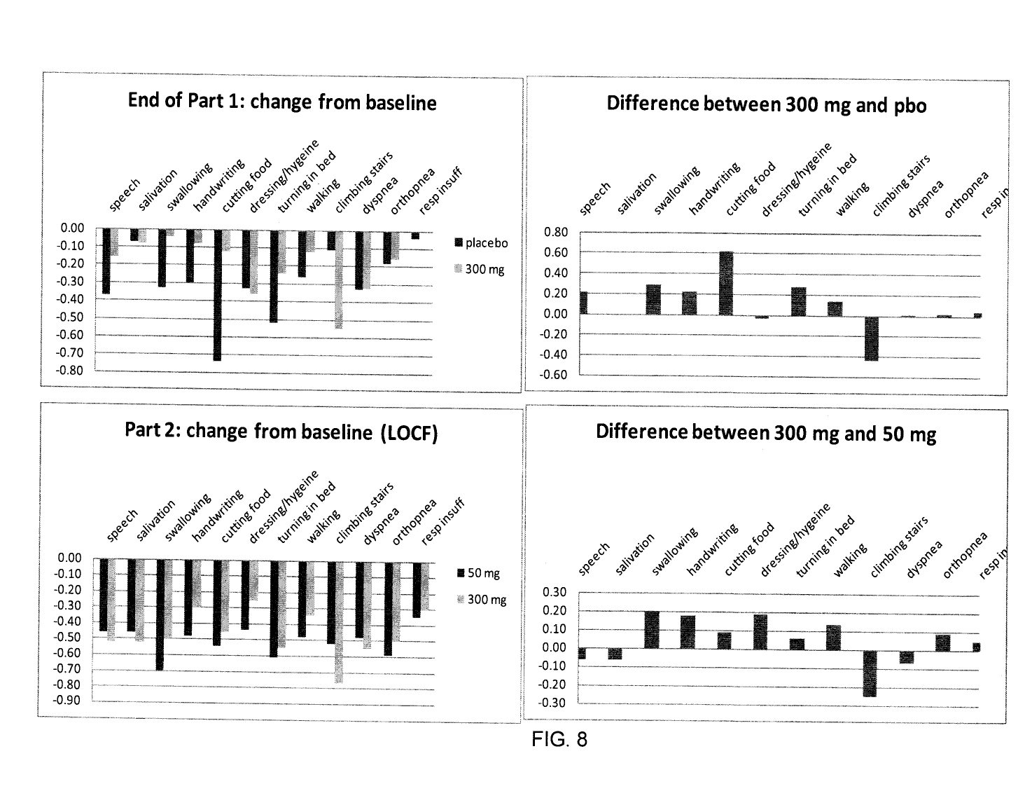

Further analysis of the ALSFRS-R subdomain results indicate that particular behaviors associated with each subdomain may be improved as a result of dexpramipexole administration. As illustrated in FIG. 4, behaviors associated with fine motor skills showed dose dependent improvement over baseline in patients who were treated with dexpramipexole and, in particular, 300 mg/day of dexpramipexole. As indicated in FIG. 4A, patients who received daily doses of 30 mg of dexpramipexole exhibited almost no reduction in handwriting score while patients receiving placebo or smaller daily doses of dexpramipexole showed a reduction in handwriting. Similarly, patients receiving 300 mg/day of dexpramipexole exhibited less reduction in cutting food and dressing and hygiene scores than patients receiving placebo or lower dose dexpramipexole (FIGS. 4B and 4C). As shown in FIG. 5, behaviors associated with bulbar function also exhibit a less dramatic decline in ALSFRS-R score over baseline when patients received dexpramipexole and, in particular, 300 mg/day of dexpramipexole. Of the behaviors quantified, swallowing scores appeared to be maintained better than other behaviors (FIG. 5A). Scores associated with gross motor and respiratory behaviors also follow similar trends as shown in FIGS. 6 and 7. As indicated in the charts of FIG. 8, the improvement in individual behaviors associated with the subdomains were generally improved over placebo in Part 1, and a similar trend is evident based on the data gathered during Part 2. Thus, a slower decline in ALSFRS-R score was exhibited in patients after placebo washout and re-randomization.

Dexpramipexole was safe and well-tolerated in ALS patients over 12 weeks of treatment at total daily doses of 50 mg, 150 mg, and 300 mg compared with placebo. There were no deaths or treatment-related SAEs during Part 1 of the study. All but 4 subjects in the study completed 12 weeks of treatment: 2 subjects withdrew consent and 2 subjects discontinued due to AEs. The most frequent AEs reported across active treatment groups were fall, muscular weakness, post-lumbar puncture syndrome, headache, and nausea. There were no per-treatment group differences in the incidence of AEs or in the incidence of vital sign, ECG or laboratory abnormalities that met pre-specified criteria for potential clinical significance. The primary prespecified analysis of the treatment effect on the slope of ALSFRS-R total scores was not statistically significant (p=0.1087); however, the estimated slope for the 300 mg group was improved by 31% relative to the estimated slope for the placebo group. Furthermore, meaningful differences were also observed in both the mean and median changes from baseline to endpoint in ALSFRS-R total scores between the placebo and 300 mg groups (39% and 50%, respectively). An exploratory analysis with covariate adjustment yielded a significant improvement in ALSFRS-R change at Week 12 for the 300 mg group as compared to the placebo group (p=0.0412). According to a recent survey of ALS specialty physicians, a reduction of ALSFRS-R decline of 25% is considered to be clinically significant, while a reduction of 50% is considered to be clinically very significant. The improvements in functional decline observed for the 300 mg group compared to placebo, therefore, were at or near levels that are considered by ALS specialty physicians to be a clinically very significant treatment effect. Such a result was unexpected in a small study of only 12 weeks duration, since the typical study design to detect an effect on clinical status in ALS has utilized large numbers of subjects (˜200 per arm) treated for 12 months' duration. There were no meaningful differences in the change from baseline to endpoint in VC or McGill QOL scores across treatment groups. Pharmacokinetic analyses demonstrated linear pharmacokinetics across the range of doses tested and PK estimates of clearance, volume of distribution, and t_{1/2} were comparable in ALS patients compared with estimates based on data from healthy adult volunteers. Results of this study demonstrate that dexpramipexole is safe and well-tolerated in subjects with ALS over 12 weeks of treatment at doses up to 300 mg per day, and further suggest that dexpramipexole may have the potential to slow functional decline in ALS as measured by the ALSFRS-R.

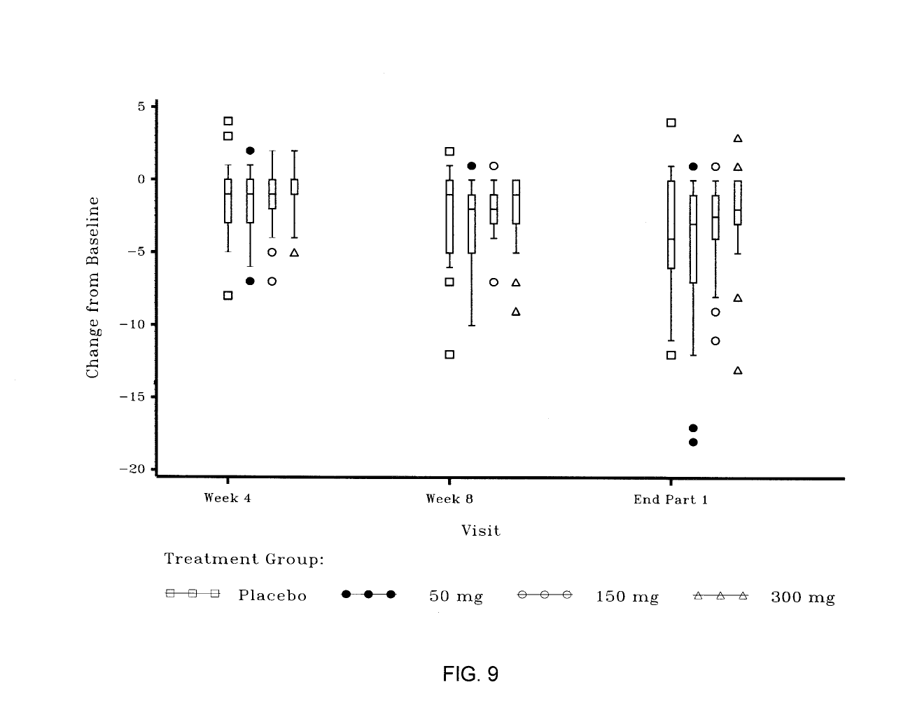

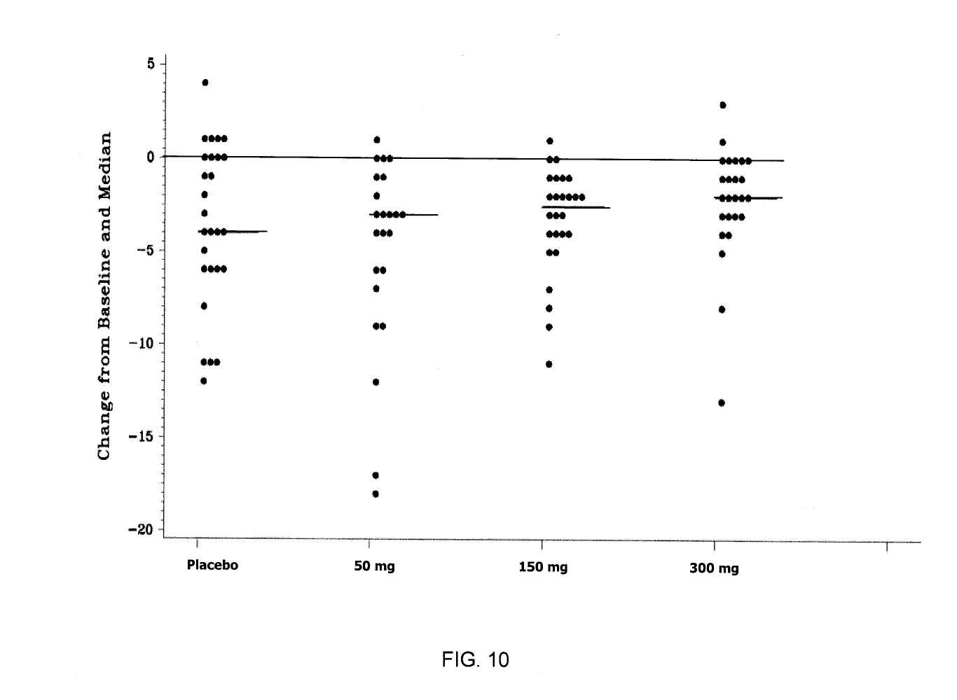

Dose related changes in the symptoms of ALS were tracked throughout the study using the ALS Functional Rating Scale, Revised (ALSFRS-R). The ALSFRS-R, scored 0 48, is used to evaluate overall functional status of ALS patients in clinical trials as well as in clinical practice. FIG. 9 shows a box plot of the results of ALSFRS-R total score of subjects taken at 4 week intervals for each treatment group. FIG. 10 shows the change from baseline for each subject in each treatment group as indicated on the x-axis with lines indicating the median score for the group and with baseline as indicated by 0. These data show that mean/median change from baseline to the endpoint of the 12 week study were −3/6/−4.0 for placebo, −5.0/−3.0 for the 50 mg treatment group, −3.3/−2.5 for the 150 mg treatment group, and −2.2/−2.0 for the 300 mg treatment group. Thus, relative to the placebo group, the 300 mg treatment group showed a 39% improvement in mean ALSFRS-R change from baseline to endpoint and a 50% improvement in median ALSFRS-R change from baseline to endpoint, as graphically illustrated in FIG. 11. This dose-related improvement in ALSFRS-R over the 12 week study suggests that daily doses of greater than about 300 mg of dexpramipexole may slow the rate of ALS symptom progression including, for example, motor function loss.

### Example 2

As shown in the Table 3, the number of patients by treatment group in Part 1 who experienced a weight loss exceeding 7% compared to baseline levels, a criterion pre-specified in the study as an adverse event. Of the six study subjects meeting this criterion, five received either placebo or 50 mg/day of dexpramipexole, the lowest dose tested, while only one patient in the higher dose groups met the excessive weight-loss criterion.

TABLE 3
Weight Loss in ALS Patients Treated with dexpramipexole

| | | dexpramipexole | dexpramipexole | dexpramipexole |
|---|---|---|---|---|
| | Placebo | 50 mg | 150 mg | 300 mg |
| Body | 3/26 (11.5%) | 2/22 (9.1%) | 0/24 (0.0%) | 1/25 (4.0%) |
| Weight | | | | |

### Example 3

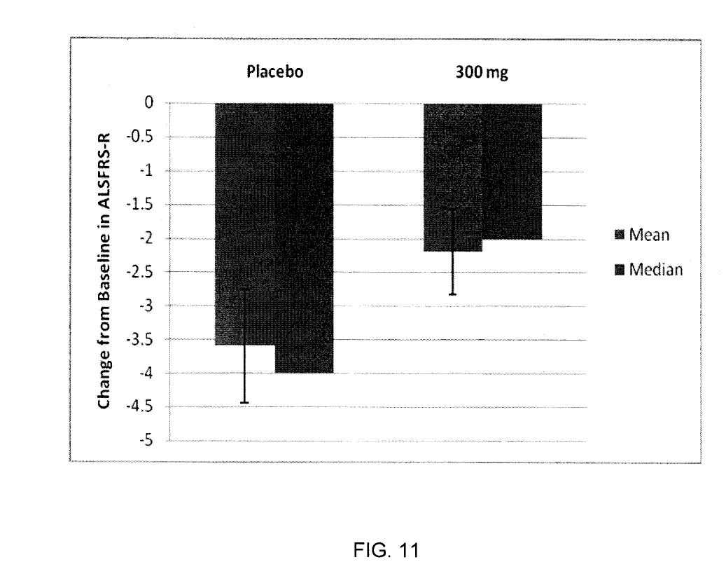

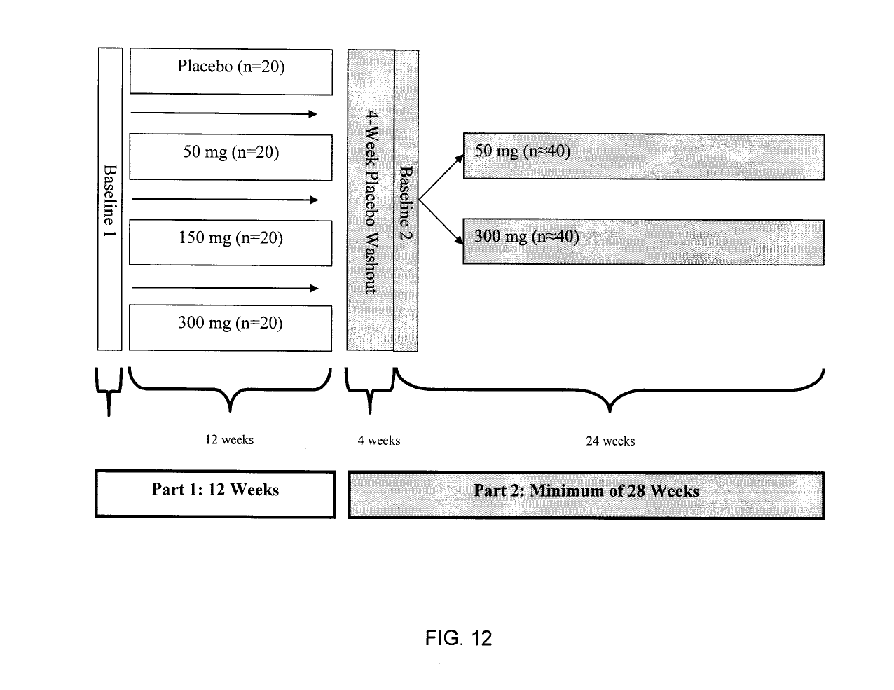

Subjects completing Part 1 (as set-forth in EXAMPLE 1) were eligible to continue into Part 2 of the study. Part 2 was a randomized, double-blind, 2-arm, parallel-group, extension study evaluating the longer-term safety, tolerability, and clinical effects of oral administration of 2 dosage levels of dexpramipexole (50 mg and 300 mg). After the conclusion of Part 1, a 4-week, single-blind, placebo washout period was carried out. The subjects were then re-randomized to 1 of 2 daily dosage levels of dexpramipexole (50 mg or 300 mg) and treated in Part 2 for up to 72 weeks. Based on the preliminary evidence of a treatment effect at 300 mg/day from Part 1, subjects active at the time of trial closure were offered the opportunity to continue receiving open-label high-dose dexpramipexole (300 mg/day) in a safety extention protocol. A study schematic for Part 1 and Part 2 of the study is presented in FIG. 12.

The transition into Part 2 of the study was expected to occur at the conclusion of the Part 1, Week 12 visit; therefore, the Part 1, Week 12 assessments did not need to be repeated at the beginning of Part 2 and these assessments served as baseline for the placebo washout period. At the beginning of Part 2, all subjects participated in a single-blind (subject blind), 4-week washout period, during which all subjects received placebo and were observed for withdrawal effects. During the washout, subjects were instructed to continue to take their study medication approximately every 12 hours and to withhold dosing on the morning of the Week 4 predose visit in Part 2. Prior to the Week 4 visit, subjects were contacted and reminded to withhold their dosing on the morning of the Week 4 (Baseline) Visit.

Following the completion of the 4-week placebo washout period, subjects were re-randomized in a 1:1 fashion to 1 of 2 dexpramipexole treatment groups: low-dose (25 mg twice per day) or high-dose (150 mg twice per day) in a double-blind manner. Prior to study drug administration, clinical assessments were performed in the following order: McGill SIS, adverse event information, ALSFRS-R, and upright VC; physical examination, including body weight, was performed and vital signs were measured; 12-lead ECG was performed; blood and urine samples were collected for safety laboratory assessments; lithium screen was performed in all subjects and serum pregnancy tests were performed for females of childbearing potential; and information on concomitant medications was collected. After all baseline predose procedures were completed, subjects took 1 dose (2 tablets) of active study drug. Following the first dose of study drug, adverse event information was collected. Approximately 2 hours (±20 minutes) after study drug administration, vital signs were measured and a 12-lead ECG was performed. Subjects were dispensed outpatient study drug, with instructions to take the second dose approximately 12 hours after the first dose on the day of the Week 4 visit. Subjects were instructed to take a dose of study drug at approximately the same time of day each morning and again 12 hours later in the evening through the remainder of the study. Subjects remained blinded to study treatment throughout the entire study.

After the Part 2 Baseline visit, clinic visits were scheduled at Week 6, Week 8, Week 12, Week 20, Week 28, Week 40, Week 52, Week 64, and Week 76; visits were to occur within 3 to 5 days of the target visit date. At all clinic visits, adverse event and concomitant medication information was collected, vital signs were measured, a 12-lead ECG was performed, and blood and urine samples were collected for safety laboratory assessments. In addition, at all clinic visits after Week 6, clinical assessments (McGill SIS, ALSFRS-R, upright VC), physical examination including body weight, serum pregnancy tests for females of childbearing potential, and lithium screen were performed; additional outpatient study drug was dispensed (except Week 76); and drug compliance was calculated. At Weeks 16, 24, 34, 46, 58, and 70, subjects were contacted by telephone. During the telephone contacts, the McGill SIS and ALSFRS-S were completed, and adverse event information was collected; in addition, at Weeks 34, 46, 58, and 70, serum pregnancy tests for females of childbearing potential were to be collected and analyzed by a local laboratory, with results submitted to the clinical site. At Week 28 (or early termination), plasma samples were collected for protein biomarker analysis.

During Part 2 of the study, randomized subjects received 2 tablets orally twice daily (25 mg or 150 mg dexpramipexole) for up to 76 weeks. Dexpramipexole was administered as a solid white, unmarked round tablet with concave edges at the top and bottom. The placebo tablets used during the placebo washout period were visually indistinguishable from the active tablets. Dose strengths for active drug tablets in Part 2 were 25 mg and 150 mg. Dosage levels were expressed in terms of the di-hydrochloride salt (i.e., an adjustment of approximately 6% was made to account for the weight of the monohydrate in the final salt form). The solid tablet formulation contained the following inactive ingredients (listed in order of percent volume): microcrystalline cellulose, mannitol, crospovidone, and magnesium stearate (vegetable source).

In Part 2, study drug was dispensed at baseline which was the same vist as the Part 1 Week 12 visit (beginning of the placebo washout period), at Week 4 (end of placebo washout), Week 8, Week 12, Week 20, Week 28, Week 40, Week 52, and Week 64.

Any medication or supplement the subject used other than the study drug specified in the protocol was considered a concomitant medication whether it was a prescription medication or over-the-counter product. The use of concomitant medications during this study was recorded throughout Part 2 of the study. All concomitant medications were recorded in the subject's source document and on the CRFs. Co-administration of other dopamine agonist medication(s) was not allowed during the trial.

Subjects taking concomitant Rilutek® (riluzole) at study entry were to be on a stable dose for 2 months prior to Day 1 of Part 1 and to continue taking the same dose throughout the study (unless it was determined that riluzole should be discontinued for medical reasons, in which case it was not to be restarted). Any planned dosage adjustment of riluzole was to be discussed in advance to determine continued eligibility for this study. Subjects who previously discontinued riluzole could have been enrolled into the study, but a washout period of 1 month was required prior to randomization.

The use of vitamins, minerals, and supplements was monitored throughout the study. The daily intake of all vitamins and supplements used during the study was to be stabilized at least 14 days prior to Day 1 of Part 1. The supplements listed below were subject to the specified dose limits and doses were to remain stable for at least 14 days prior to Day 1 of Part 1, and throughout the study: CoQ10≦600 mg/day, Creatine≦5 g/day, Vitamin E≦1000 IU/day, and Vitamin C≦1000 mg/day. The daily dose limits above included doses obtained through the use of multivitamins and supplements.

Throughout the study, subjects were monitored closely for the observation of unexpected or clinically significant safety or tolerability events. Safety evaluations included physical examination, neurological examination, vital signs, 12-lead ECG, laboratory evaluations, lithium screening, and monitoring of adverse events. Vital signs, including systolic and diastolic blood pressure, respiratory rate, pulse rate, and temperature, were measured after the subject had rested for 5 minutes. The following guidelines were used to grade the intensity of an AE:
Mild The event was of little concern to the subject and/or of no clinical significance. The event was not expected to have any effect on the subject's health or well-being.
Moderate The subject had enough discomfort to cause interference with or change in usual activities. The event was of some concern to the subject's health or well-being. The event may have required medical intervention.
Severe The subject was incapacitated and unable to work or participate in many or all usual activities. The event was of definite concern to the subject or posed substantial risk to the subject's health or well-being. The event was likely to require medical intervention or close follow-up.

Interviews for AEs were to be conducted often throughout the course of the study. At a minimum, such interviews were to occur during each subject visit, including telephone contacts. The interview for AEs was to be conducted early during a given subject interaction. This was especially important when the functional rating scale (ALSFRS-R) was being administered during the same visit. During such visits, the AE interview was to be conducted prior to administration of the ALSFRS-R.

The ALSFRS-R, VC, and McGill QoL-SIS scores were summarized by treatment group with the rate of change estimate derived from a linear mixed-effects model. Linear decline of the ALSFRS-R over time has been shown previously. If the linearity assumption did not hold (quadratic term with a p-value<0.05), a repeated measures mixed-effect model was to be used. A mixed-model analysis was used to fit a model that included time, treatment group, and the interaction between time and treatment group simultaneously. The coefficient of time (the slope, or rate of change) estimated for each treatment group was used to test for differences between the treatment groups. Coefficient of time estimate along with its standard error was reported.

An additional sensitivity analysis was performed, based on a rank score derived from a joint ranking of mortality (time to mortality) and functional decline for surviving subjects (change from baseline in ALSFRS-R) using the methodology proposed by Finkelstein and Schoenfeld. A subject's score (ranking) was calculated by comparing each subject to every other subject in the trial, setting a score of +1 if the outcome was better than the subject being compared, −1 if worse and 0 if tied. The subject's rank (score) was then calculated by summing up his comparison to all the other subjects in the study. For this comparison, a subject who died earlier than the comparator subject was given a comparison score of −1; if 2 subjects completed the study, their comparison score was based on a comparison of their ALSFRS-R change values at the end of the study; if a subject discontinued early, his comparison to each other subject was based on the comparison of their ALSFRS-R change at the latest time point at which they both had an ALSFRS-R value. This resulted in subjects who died getting the worst scores (ranks) and being ranked according to the time of death; subjects who survived were ranked above the deaths and in general were ranked according to their endpoint ALSFRS-R change value, with special handling to rank early discontinuations as described above.

For the double-blind, active-treatment period of Part 2, the Kaplan-Meier estimates of median time to death or tracheostomy and 95% confidence intervals, and the 25^{th} and 75^{th} quartiles and 95% confidence intervals were presented for each treatment group. The comparison between the 2 treatment groups was performed using a log rank test. A figure of the Kaplan-Meier estimated curve for each treatment group was also presented. The number and percentage of subjects who were hospitalized for tracheostomy or died or were censored were tabulated. If an insufficient number of events occurred, only the tabulation of subjects who were hospitalized for tracheostomy, died, or were censored was to be presented. For the double-blind, active treatment period of Part 2, the Kaplan-Meier estimates of median time to NIV for >22 hours/day for >10 consecutive days or tracheostomy or death and 95% confidence interval, the 25^{th} and 75^{th} quartiles and 95% confidence intervals were presented for each treatment group. Time to AV or tracheostomy or death was analyzed similarly to time to death or tracheostomy. Only subjects in the ITT Population who did not have feeding placement at baseline were included for this analysis. For the double-blind, active treatment period of Part 2, the time to feeding tube placement was to be analyzed similarly to time to death or tracheostomy. If an insufficient number of events occurred, only the tabulation of subjects who had feeding tube placement or who were censored was to be presented.

Duration of dosing in days and mean daily dose in mg were summarized by treatment group using descriptive statistics for each study period of Part 2. Percent compliance for the double-blind, active-treatment period of Part 2 was summarized by treatment group using descriptive statistics and the number and percent of subjects with compliance<80%, 80-100%, and >100%.

The SAP specified that the analysis of clinical status evaluation data would be conducted on the ITT population where the ITT population consisted of data from all subjects in the safety population for whom at least 1 post baseline clinical status evaluation (McGill SIS, ALSFRS-R, or VC) was obtained. In the SAP, the analysis of time to death or tracheotomy was listed under clinical status evaluation data, which would imply that this analysis be carried out on the ITT population. However, 1 subject in the 50 mg group died after 28 days of follow-up without an evaluation for McGill SIS, ALSFRS-R, or VC. Since it was inappropriate to exclude from the survival analysis any randomized treated subjects who died during the follow-up period; the analysis of survival, time to death or tracheotomy, and the joint rank analysis that combined time to death and change from baseline in ALSFRS-R were conducted on the safety sample, 48 subjects in the 50 mg group and 44 subjects in the 300 mg group.

A total of 97 subjects who completed Part 1 of the study were entered into the placebo washout period of Part 2. Enrollment by site at the 20 participating sites ranged from a minimum of 1 subject to a maximum of 9 subjects. Five (5) subjects discontinued early from the placebo washout period: 1 subject withdrew consent, 1 subject was lost to follow-up, and 3 subjects died due to ALS. Ninety-two (92) subjects completed the placebo washout period and entered the double-blind treatment period

A total of 92 randomized subjects took at least 1 dose of study drug during the double-blind treatment period. Forty-eight (48) subjects were randomized to 50 mg dexpramipexole and 44 subjects were randomized to 300 mg dexpramipexole. Seventy-one (71) subjects completed the study through Week 28. Twenty-one (21) subjects, 14 subjects in the 50 mg group and 7 subjects in the 300 mg group, discontinued from the study prior to Week 28. The most common reasons for discontinuing early were ALS-related death (8 subjects) and withdrawal of consent (7 subjects).

It should be noted that only subjects who died on-treatment are included as a “death” in the disposition table. During the first 24-week randomized active treatment period, the total number of deaths was 7 in the 50 mg group versus 2 in the 300 mg group. Three additional subjects died after completing the Week 28 visit. An additional 6 subjects died after discontinuing the study, most of whom withdrew consent due to their inability to travel to the study center for visits.

During the double-blind treatment period, all 92 randomized subjects took at least 1 dose of active study medication and were included in the Safety population. Ninety of the 92 randomized subjects had at least 1 post-baseline clinical status evaluation and were included in the ITT population. Two subjects in the 50 mg group were missing all post-baseline clinical status evaluations and were excluded from the ITT population.

The medications used at baseline of the placebo washout period (Part 1, Week 12) were consistent with the age and ALS diagnosis of the population under study. At baseline, 96 (99%) subjects were receiving one or more medications. WHO drug classes used by ≧20.0% of subjects overall included Vitamins (64%), Other Nervous System Drugs (58%), Psychoanaleptics (40%), Anti-inflammatory and Antirheumatic Products (31%), Other Alimentary Tract and Metabolism Products (31%), Antithrombotic Agents (27%), Analgesics (26%), Lipid Modifying Agents (24%), and Psycholeptics (24%). Fifty-six subjects (58%) were taking concomitant riluzole at baseline. Other common concomitant medications during the placebo washout period were tocopherol (31%), ubidecarenone (29%), and ascorbic acid (27%).

At baseline of the double-blind treatment period (Part 2, Week 4), 91 (99%) subjects were receiving one or more medications. WHO drug classes used by ≧20.0% of subjects overall included Other Nervous System Drugs (59%), Vitamins (59%), Psychoanaleptics (41%), Anti-inflammatory and Antirheumatic Products (30%), Other Alimentary Tract and Metabolism Products (28%), Antithrombotic Agents (27%), Analgesics (25%), Psycholeptics (25%), Lipid Modifying Agents (23%), Muscle Relaxants (23%), Agents Acting on the Renin-Angiotensin System (21%), and Urologicals (20%). Fifty-four subjects (59%) were taking concomitant riluzole at baseline; concomitant riluzole use was 52% in the 50 mg group and 66% in the 300 mg group.

Subjects were highly compliant with study drug during the double-blind treatment period. Median compliance through Week 28 was 99.0% in the 50 mg group and 98.2% in the 300 mg group (TABLE 15). Twenty-two subjects (11 in each group) had compliance>100%. Compliance through the end of the study was similar to that through Week 28.

Each item of the ALSFRS-R was scored on a 4 to 0 scale, with a 4 indicating normal function and each lower number indicating progressive worsening of function. For change from baseline, therefore, a score of zero would indicate no loss of function and increasingly negative scores would indicate greater losses of function.

At baseline of the placebo washout period (Week 12 of Part 1), the ALSFRS-R total scores were similar in the 4 Part 1 treatment groups, with mean scores of 35.0, 32.4, 35.8, and 36.2 for the placebo, 50 mg, 150 mg, and 300 mg groups, respectively, and median scores ranging from 34 to 37. Over the 4 weeks of the placebo washout period, the mean change from baseline in these groups was −1.5 (placebo), −0.7 (50 mg), −1.0 (150 mg), and −1.5 (300 mg). For all subjects combined during the placebo washout period (N=92), the mean baseline value was 34.9, and the mean and median changes from baseline to the end of the 4-week placebo washout were −1.2 and −0.5, respectively.

At baseline of the placebo washout period (Week 12 of Part 1), mean values for upright VC in the 4 Part 1 treatment group were 78.5%, 82.5%, 82.3%, and 82.1% for the placebo, 50 mg, 150 mg, and 300 mg groups, respectively. Median values were similar in the placebo, 50 mg, and 150 mg groups (range: 80.9 to 82.7%); median upright VC in the 300 mg group was 91.2%. Over the 4 weeks of the placebo washout period, the mean change from baseline in VC in these groups was −5.1% (placebo), −2.9% (50 mg), −1.7% (150 mg), and −2.7% (300 mg). For all subjects combined during the placebo washout period (N=92), the mean upright VC at baseline was 81.3%, and the mean and median changes from baseline to the end of the placebo washout were −3.1% and −3.5%, respectively.

Subjects rated their quality-of-life on a scale of 0 (very bad) to 10 (excellent), using the McGill SIS. Decreases from baseline indicate deterioration of the subject's quality of life. At baseline of the placebo washout period (Week 12 of Part 1), the McGill SIS scores varied across the 4 treatment groups, with the lowest mean score in the 50 mg group (6.3) and the highest mean scores in the placebo and 300 mg groups (7.3). For all subjects combined during the placebo washout period (N=92), the mean baseline value was 6.9, and the mean and median changes from baseline to the end of the placebo washout were −0.3 and 0.0, respectively.

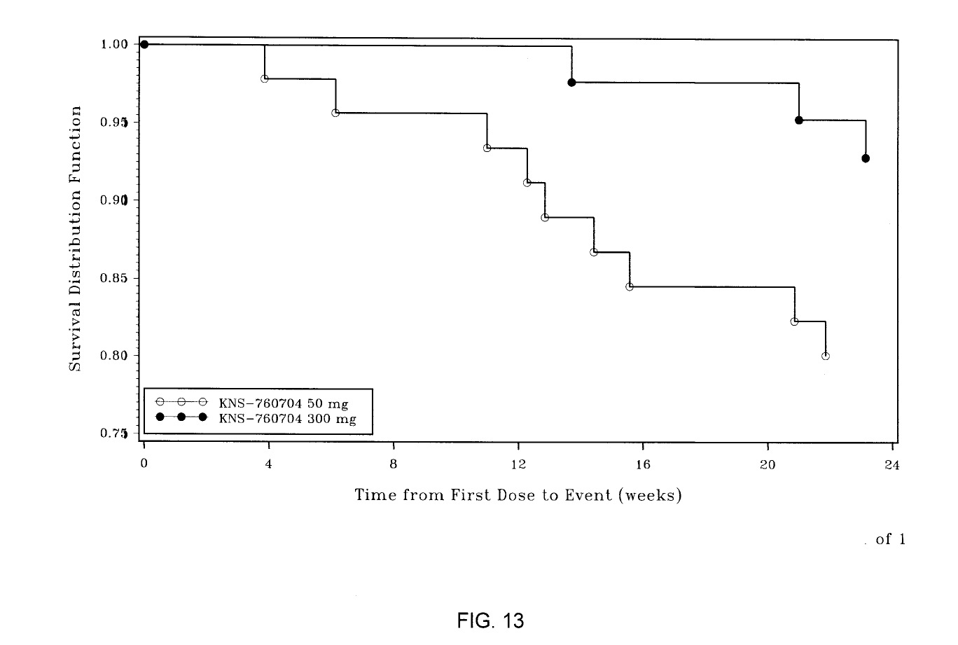

Survival analyses were performed on the Safety population, rather than the ITT population, in order to include all subject deaths. None of the subjects required tracheostomy through Week 28 of the double-blind treatment period. In the double-blind treatment period through Week 28, 9 (19%) subjects in the 50 mg group and 3 (7%) subjects in the 300 mg group died. Thus, 81% of the 50 mg group and 93% of the 300 mg group did not require tracheostomy and did not die. Based on a log rank test, the difference between the 2 treatment groups in time to death approached statistical significance (p=0.0708). It should be noted the all deaths during Part 2, including deaths that occurred after discontinuation from the study, were counted in the Kaplan-Meier estimates. FIG. 13 provides a graphic presentation of the Kaplan-Meier estimates for the time to tracheostomy or death through Week 28.

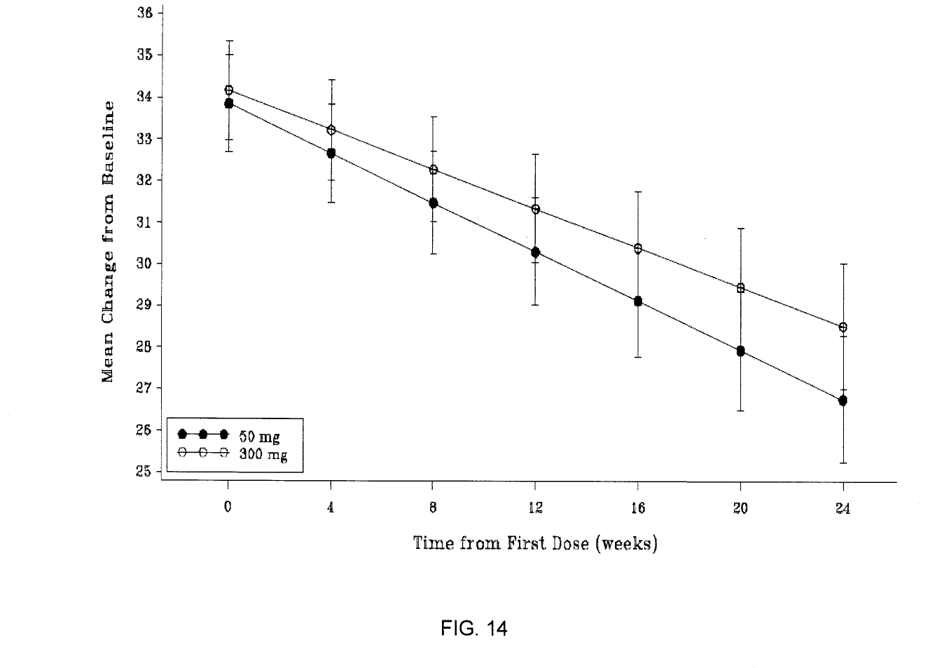

The test of linearity for the analysis of ALSFRS R scores in Part 2 resulted in a non-significant quadratic term; therefore, the linear mixed effects model was used as the primary analysis. At baseline of the double-blind treatment period (Week 4 of Part 2), the ALSFRS-R total scores were similar in the 2 treatment groups, with a median score of 35 in both treatment groups, and mean scores of 34.0 in the 50 mg group and 33.8 in the 300 mg group. Starting at Week 8 and continuing through Week 28, the mean change from baseline in ALSFRS-R total scores was attenuated in the 300 mg group compared with the 50 mg group; the mean change was −6.5 in the 50 mg group and −6.2 in the 300 mg group. The treatment group difference in mean change scores are a biased estimate of the true treatment group difference due to the larger number of deaths and dropouts in the 50 mg group than in the 300 mg group. A more appropriate estimate of treatment group difference is provided by the slopes estimates as specified in the SAP. The slope estimates of ALSFRS-R scores from the linear mixed effects model through Week 28 of the study were −1.283 for the 50 mg group and −1.021 for the 300 mg group. This corresponds to a relative reduction of 20.4% in the rate of decline in ALSFRS-R scores for the 300 mg group relative to the 50 mg group over 24 weeks of treatment (p=0.1778). A plot of the mean (SE) ALSFRS-R total scores estimated from the linear mixed effects model for slope is shown in FIG. 14.

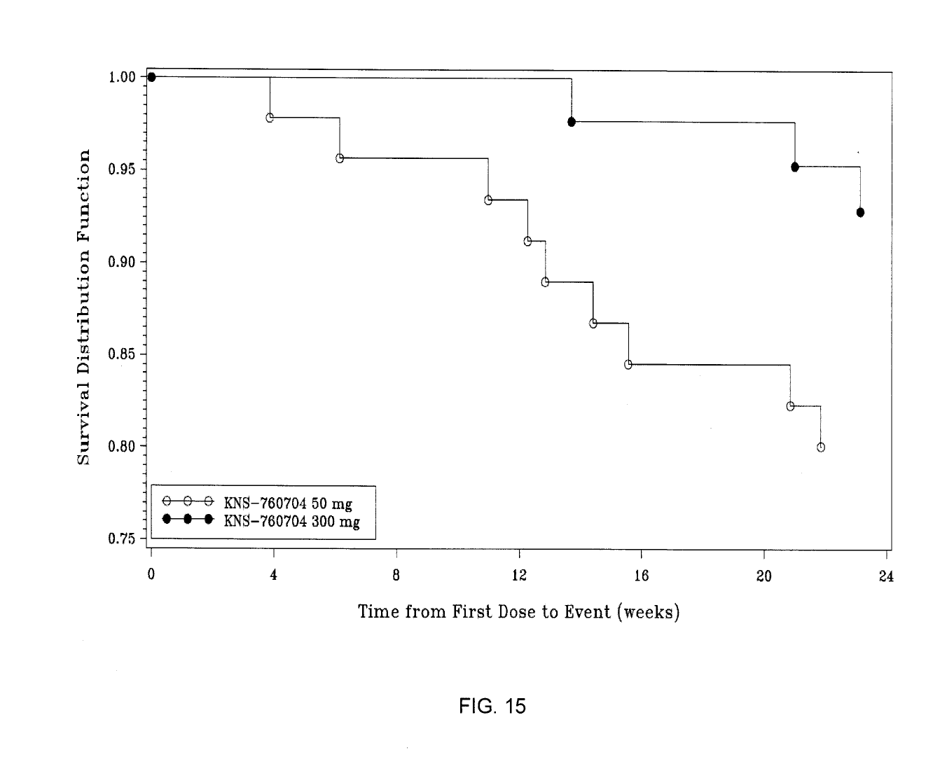

When deaths are unevenly distributed between the treatment groups, even the mixed effects slopes model may not adequately account for the effect of deaths in the estimate of the treatment effect. For this reason, the SAP specified as a sensitivity analysis a generalized Gehan Wilcoxon rank test based on a joint ranking of time to survival and change from baseline in ALSFRS-R score. Analysis of frequency and time to death was described by Kaplan-Meier life-table estimates of survival time, for which treatment group differences were analyzed by log rank test. There were a total of 9 deaths in the 50 mg group and 3 deaths in the 300 mg group during the double-blind treatment period through Week 28 (p=0.0708; FIG. 15), which includes 2 subjects in the 50 mg group and 1 subject in the 300 mg group who died after discontinuing study medication but prior to Part 2, Week 28.

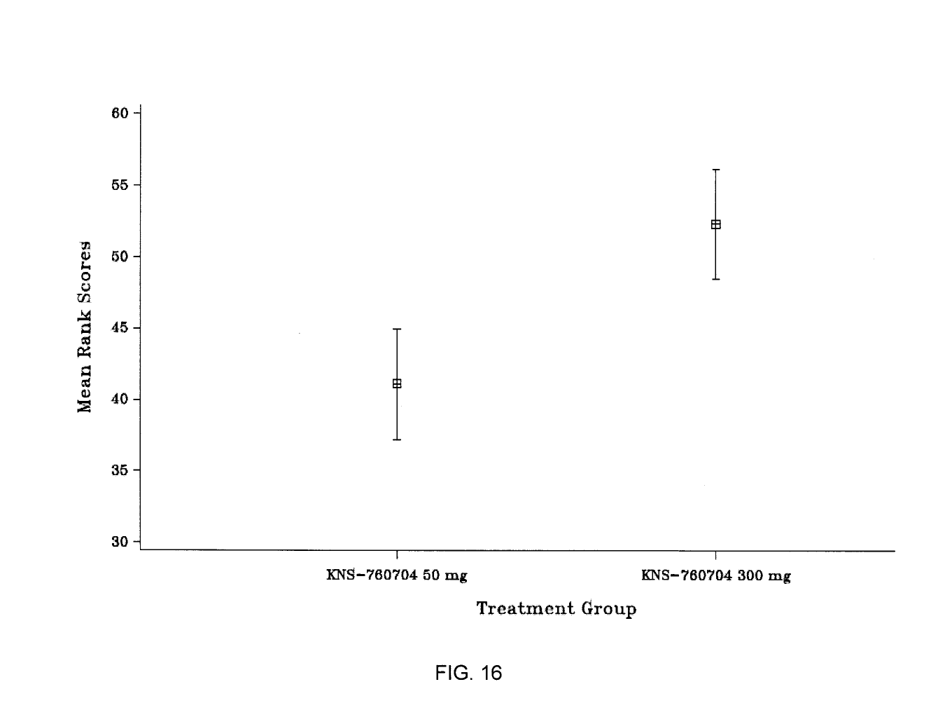

A joint-rank test of survival and ALSFRS-R data was conducted to compare the global clinical outcomes between the 2 treatment groups. A statistically significant difference in the joint rank test (generalized Gehan Wilcoxon test) was observed for the 50 mg group versus the 300 mg group through Week 28 (p=0.046). When an analysis of covariance (ANCOVA) was run on the ranks to adjust for baseline variables, the statistical significance of this difference was increased (p=0.0115). The covariates in the ANCOVA included baseline ALSFRS-R score, time from symptom onset, site of disease onset, and concomitant use of riluzole. The first 3 covariates were chosen based on stepwise regression to select variables associated with the ranks and concomitant use of riluzole was included because of its potential confounding effect. FIG. 16 shows plots of the mean rank of joint scores for the combined time to death and changes from baseline in ALSFRS-R total scores.

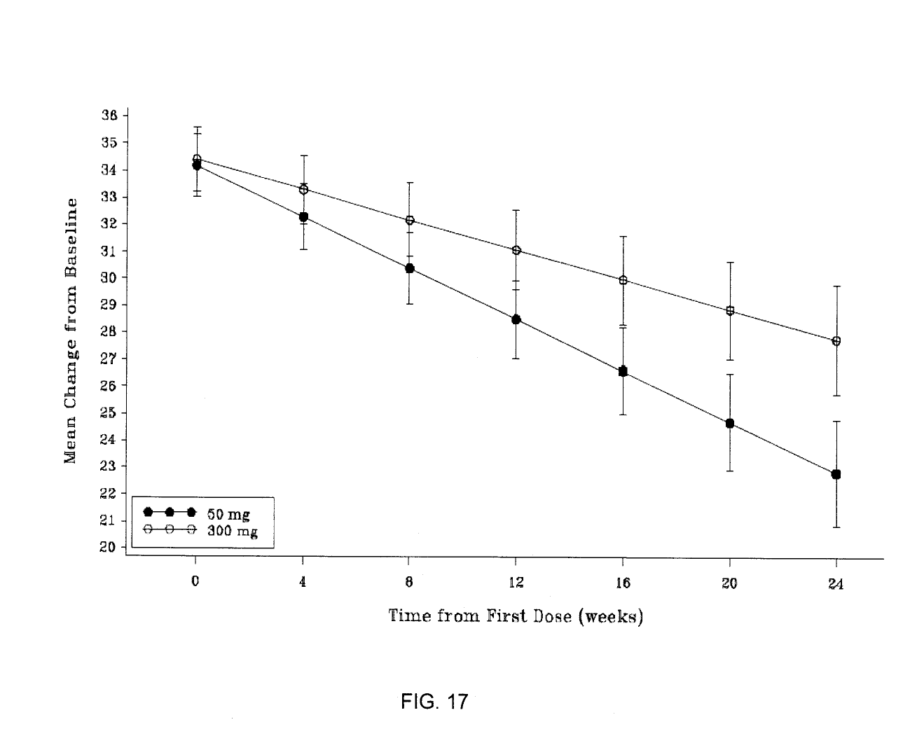

Imputing an ALSFRS-R score of zero for the first scheduled visit after the time of death is an alternative method for adjusting the linear mixed-effects slopes model for the impact of death outcomes. This method was not prespecified in the SAP but has been used by other ALS studies. Because of the large imbalance in deaths during the randomized double-blind treatment period (in favor of the 300 mg group), the resulting impact on the slopes of the 2 groups was −2.05 in the 50 mg group versus −1.19 in the 300 mg group, a reduction in decline of 42% (p=0.018; FIG. 17).

Another sensitivity analysis prespecified in the SAP was the repeated measures mixed effect model with a comparison of the 2 treatment groups at Week 28. Based on estimates from this model, the 300 mg group had a 19.7% smaller decline in ALSFRS-R scores than the 50 mg group (−5.66 versus −7.05, p=0.345). This model with the primary comparison of the treatment groups at Week 28 does not adequately account for the effect of the higher early death rate in the 50 mg treatment group. An alternative statistical test to compare the treatment groups within the context of the repeated measures mixed effect model is the overall difference in mean ALSFRS-R scores averaged across all visits, this test resulted in favor of the 300 mg group.

There was no effect of riluzole in Part 2 on either slope of ALSFRS R total score or mortality, or on the ranks determined jointly from survival and change in ALSFRS-R score. ALSFRS-R total scores were also assessed through the end of the study. Similar to findings during the first 24 weeks of active treatment, the mean change from baseline in ALSFRS-R total scores was attenuated in the 300 mg group compared with the 50 mg group at each assessment through the end of the study. The mean values past Week 28 underestimate the treatment group difference due to the differential rates for death and dropout in the treatment groups and are compromised by the smaller number of subjects and loss of follow-up data due to the administrative closure of the study after the last subject completed Week 28. The treatment group differences in mean ALSFRS-R Domain scores are biased underestimates of the true treatment group differences due to the larger number of deaths and dropouts in the 50 mg group than in the 300 mg group.

At baseline of the double-blind treatment period (Week 4 of Part 2), mean values for upright VC were 76.7% in the 50 mg group and 81.7% in the 300 mg group, a baseline imbalance between the 2 groups of 5 points (TABLE 4). The mean change from baseline to Week 28 in upright VC was −12.4% in the 50 mg group and −15.1% in the 300 mg group; median changes were −10.4% and −11.5%, respectively. A summary of mean and median change from baseline to Weeks 8, 12, 20, and 28, and the endpoint of Part 2 in upright vital capacity is presented in TABLE 4.

TABLE 4
Mean Change from Baseline of Part 2 in Upright Vital Capacity-
Double-Blind Treatment Period (ITT Population)

| | dexpramipexole (total daily dose) | |
|---|---|---|
| | 50 mg | 300 mg |
| Upright Vital Capacity (% predicted) | (N = 46) | (N = 44) |
| Baseline (Part 2: Week 4 Pre-dose) | (N = 45) | (N = 42) |
| Mean (SE) | 76.7 (2.81) | 81.7 (3.27) |
| Median | 78.4 | 84.1 |
| Minimum, maximum | 31, 117 | 30, 120 |
| Week 8 | (N = 45)^{a} | (N = 42)^{a} |
| Mean (SE) | 73.7 (2.92) | 80.3 (3.20) |
| Mean Δ (SE)^{a} | −3.2 (1.67) | −1.4 (0.91) |
| Median Δ | −1.9 | −0.8 |
| Minimum, maximum Δ | −36, 35 | −14, 19 |
| Week 12 | (N = 41)^{a} | (N = 40)^{a} |
| Mean (SE) | 72.9 (3.82) | 78.5 (3.34) |
| Mean Δ (SE)^{a} | −4.0 (2.19) | −4.2 (1.91) |
| Median Δ | −3.1 | −4.3 |
| Minimum, maximum Δ | −44, 44 | −34, 37 |
| Week 20 | (N = 33) | (N = 40)^{a} |
| Mean (SE) | 75.8 (4.02) | 70.8 (3.77) |
| Mean Δ (SE)^{a} | −6.0 (3.36) | −10.6 (1.58) |
| Median Δ | −7.2 | −9.0 |
| Minimum, maximum Δ | −34, 83 | −36, 8 |
| Week 28 | (N = 33) | (N = 36)^{a} |
| Mean (SE) | 69.5 (3.79) | 67.3 (4.44) |
| Mean Δ (SE)^{a} | −12.4 (3.00) | −15.1 (2.63) |
| Median Δ | −10.4 | −11.5 |
| Minimum, maximum Δ | −48, 37 | −56, 14 |
| Endpoint of Part 2 | (N = 45)^{a} | (N = 42)^{a} |
| Mean (SE) | 62.2 (4.19) | 61.1 (3.94) |
| Mean Δ (SE)^{a} | −14.6 (3.54) | −20.3 (2.88) |
| Median Δ | −11.5 | −14.3 |
| Minimum, maximum Δ | −67, 73 | −64, 14 |

SE = standard error
^{a}One additional subject provided visit data but did not have a baseline value to calculate change.

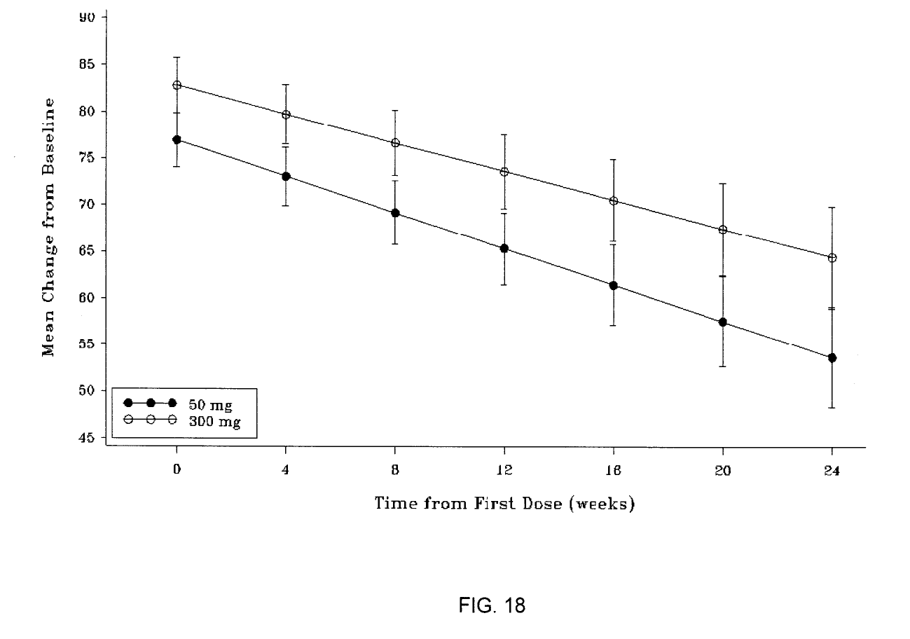

The linear mixed effects model estimates for slopes of the 2 groups in change from baseline in vital capacity were −2.452 and −3.067 for the 50 mg and 300 mg groups, respectively; based on this model, the slope of upright vital capacity did not differ significantly between treatment groups (p=0.4025). However, the vital capacity slope estimates from this model do not appropriately account for subjects who died during Part 2 through Week 28. When 0 values are imputed for the first post-death visit of subjects who died in the 2 groups, the resulting slope estimates for the 2 groups were −4.20 and −3.33 for 50 mg and 300 mg, respectively, which represents a 21% attenuation of decline in vital capacity for the 300 mg group compared with the 50 mg group (FIG. 18).

The CRF design for collection of vital capacity data required that both raw vital capacity data (measured VC) and calculated/derived vital capacity data (Predicted Normal, % Predicted, and % Variability) be manually recorded on the CRF. As part of the QC of study data, the values for Predicted Normal, % Predicted and % Variability were electronically re-calculated and compared to those data entered by the site. This review revealed that much of the manually calculated/derived data recorded on the CRFs were not accurate and/or were not expressed to 1 decimal place, the format being used for the data analysis. Therefore, electronically calculated values for Predicted Normal, % Predicted, and % Variability using raw data values were used for the data analysis, rather than the manually recorded entry on the CRF by the site. The accuracy of the raw data values were verified during routine monitoring of source data comparison to the CRF entries for these data points.

At baseline of the double-blind treatment period, mean SIS scores were 6.3 in the 50 mg group and 6.9 in the 300 mg group, with a median value of 7.0 in both groups (TABLE 5). With the exception of the 300 mg group at Week 8 (mean change of 0.0), minor mean decreases were observed in both treatment groups through Week 28, with no consistent pattern. Based on a linear mixed-effects analysis, the slope of McGill SIS scores did not differ significantly across the 2 treatment groups (p=0.5876). A summary of mean and median change from baseline to Weeks 8, 12, 16, 20, 24, and 28, and the endpoint of Part 2 in the McGill SIS is presented in TABLE 5.

TABLE 5
Mean Change from Baseline in McGill SIS -Double-Blind Treatment
Period (ITT Population)

| | dexpramipexole (total daily dose) | |
|---|---|---|
| | 50 mg | 300 mg |
| McGill SIS | (N = 46) | (N = 44) |
| Baseline (Part 2: Week 4 Pre-dose) | (N = 46) | (N = 44) |
| Mean | 6.3 (0.36) | 6.9 (0.32) |
| Median | 7.0 | 7.0 |
| Minimum, maximum | 1, 10 | 2, 10 |
| Week 8 | (N = 46) | (N = 44) |
| Mean (SE) | 6.2 (0.37) | 6.9 (0.32) |
| Mean Δ (SE)^{a} | −0.2 (0.19) | 0.0 (0.21) |
| Median Δ | 0.0 | 0.0 |
| Minimum, maximum Δ | −4, 4 | −3, 4 |
| Week 12 | (N = 41) | (N = 41) |
| Mean (SE) | 6.5 (0.34) | 6.5 (0.37) |
| Mean Δ (SE)^{a} | −0.1 (0.20) | −0.4 (0.25) |
| Median Δ | 0.0 | 0.0 |
| Minimum, maximum Δ | −2, 4 | −4, 4 |
| Week 16 | (N = 39) | (N = 43) |
| Mean (SE) | 6.1 (0.37) | 6.4 (0.33) |
| Mean Δ (SE)^{a} | −0.3 (0.22) | −0.4 (0.23) |
| Median Δ | 0.0 | 0.0 |
| Minimum, maximum Δ | −3, 5 | −5, 3 |
| Week 20 | (N = 34) | (N = 40) |
| Mean (SE) | 6.1 (0.33) | 6.4 (0.34) |
| Mean Δ (SE)^{a} | −0.7 (0.22) | −0.5 (0.30) |
| Median Δ | −1.0 | 0.0 |
| Minimum, maximum Δ | −3, 3 | −8, 4 |
| Week 24 | (N = 33) | (N = 36) |
| Mean (SE) | 6.3 (0.43) | 6.3 (0.36) |
| Mean Δ (SE)^{a} | −0.4 (0.23) | −0.7 (0.30) |
| Median Δ | 0.0 | 0.0 |
| Minimum, maximum Δ | −4, 2 | −5, 2 |
| Week 28 | (N = 34) | (N = 36) |
| Mean (SE) | 5.9 (0.45) | 6.0 (0.38) |
| Mean Δ (SE)^{a} | −0.7 (0.23) | −0.9 (0.31) |
| Median Δ | 0.0 | −1.0 |
| Minimum, maximum Δ | 4, 1 | −6, 3 |
| Endpoint of Part 2 | (N = 46) | (N = 44) |
| Mean (SE) | 5.9 (0.38) | 6.1 (0.35) |
| Mean Δ (SE)^{a} | −0.5 (0.27) | −0.8 (0.30) |
| Median Δ | 0.0 | −1.0 |
| Minimum, maximum Δ | 4, 7 | −6, 4 |

SE = standard error

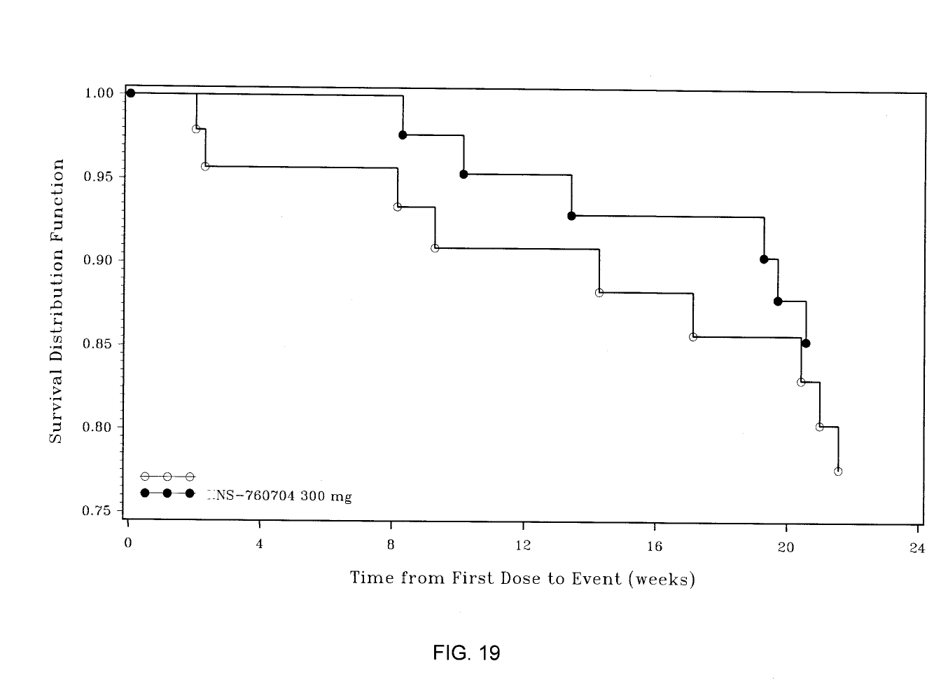

In the double-blind treatment period through Week 28, 9 (19%) subjects in the 50 mg group and 6 (14%) subjects in the 300 mg group had feeding tubes placed. Based on a log rank test, the difference between the 2 treatment groups in time to placement of a feeding tube was not statistically significant (p=0.3469). FIG. 19 provides a graphic presentation of the Kaplan-Meier estimates for the time to feeding tube placement. Time to need for assisted ventilation was not analyzed during Part 2; sites were asked whether NIV was initiated, not if NIV was necessary by an objective threshold.

As an exploratory analysis of VC upright and supine data from Part 1, correlation coefficients were calculated among the following variables: baseline upright VC, baseline supine VC, baseline difference between upright VC and supine VC, baseline ALSFRS-R total score, change from baseline to Week 12 upright VC, change from baseline to Week 12 supine VC, change from baseline to Week 12 difference between upright VC and supine VC, change from baseline to Week 12 ALSFRS-R total score.

At baseline of the placebo washout period (Part 1, Week 12), the mean ALSFRS-R total scores and upright vital capacity values were similar in the 4 Part 1 treatment groups. Over the 4 weeks of the placebo washout period, the mean change from baseline in ALSFRS-R scores was −1.5 (placebo), −0.7 (50 mg), −1.0 (150 mg), and −1.5 (300 mg). The mean change from baseline in VC was −5.1% (placebo), −2.9% (50 mg), −1.7% (150 mg), and −2.7% (300 mg). For all subjects combined during the placebo washout period (N=92), the mean and median changes from baseline to the end of the 4-week placebo washout were −1.2 and −0.5, respectively, for ALSFRS-R scores and −3.1% and −3.5%, respectively for upright VC.

The primary analysis of ALSFRS-R data was a linear mixed-effects analysis of the treatment effect on the slope of ALSFRS-R total scores during the study. The slope of ALSFRS-R scores through Week 28 was −1.283 for the 50 mg group and −1.021 for the 300 mg group, a 20.4% attenuation of the slope of decline in the high-dose group relative to the low-dose group. The primary analysis of treatment effects on the slope of ALSFRS-R scores between treatment groups was not significant (p=0.1778).

The frequency of death was higher and the time to death was shorter in the 50 mg group relative to the 300 mg group, although the difference was not statistically significant in this small study (p=0.0708). The mean differences in slopes between the groups at later visits in the study were underestimated to the extent that there was a disproportionate number of discontinuations and deaths in the 50 mg group relative to the 300 mg group.

Because of the large imbalance in deaths during the randomized double-blind treatment period (in favor of the 300 mg group), a modified linear mixed-effects model for slope of ALSRFS-R total scores was performed in which values of zero were imputed for the first post-death visit among subjects who died through Week 28. In this model, the resulting impact on the slopes of the 2 groups was −2.05 in the 50 mg group versus −1.19 in the 300 mg group, a reduction in decline of 42% (p=0.018).

A joint-rank test of survival and ALSFRS-R data was conducted to compare the global clinical outcomes between the 2 treatment groups. The results of this test were statistically significant, favoring the 300 mg group over the 50 mg group at Week 28 (p=0.046). When an ANCOVA was run on the ranks to adjust for baseline variables (baseline ALSFRS-R score, time from symptom onset, site of disease onset, and concomitant use of riluzole), the statistical significance of the difference was increased (p=0.0115).

At baseline of the double-blind treatment period (Week 4 of Part 2), mean values for upright vital capacity were 76.7% in the 50 mg group and 81.7% in the 300 mg group, a baseline imbalance between the 2 groups of 5 points. The mean change from baseline to Week 28 in upright vital capacity was −12.4% in the 50 mg group and −15.1% in the 300 mg group; median changes were −10.4% and −11.5%, respectively. The estimates of slope for vital capacity for the 50 mg and 300 mg groups through Week 28 were −2.452 and −3.067 (unadjusted), respectively, and −4.17 and −3.42 (adjusted for deaths through Week 28), respectively. For the adjusted vital capacity slopes, the 300 mg group slope was attenuated by 18% relative to the 50 mg group slope.

At baseline of the double-blind treatment period, mean SIS scores were 6.3 in the 50 mg group and 6.9 in the 300 mg group, with a median value of 7.0 in both groups. In general, minor mean decreases were observed in both treatment groups through Week 28, with no consistent pattern. Based on a linear mixed-effects analysis, the slope of McGill SIS scores did not differ significantly between the 2 treatment groups (p=0.5876).

### Safety Evaluation

Ninety-two subjects completed the placebo washout period. Five subjects prematurely discontinued. All 92 randomized subjects took at least 1 dose of the study drug and were included in the Safety Population. Median duration of treatment was 169 days in both treatment groups. A summary of duration of dosing and mean daily dose in the 2 treatment groups is presented in TABLE 6.

TABLE 6
Exposure to Study Drug-Double-Blind Treatment Period (Safety
Population)

| | |
|---|---|
| 50 mg | 300 mg |
| (N = 48) | (N = 44) |

Through Week 28

| | | |
|---|---|---|
| Duration of dosing^{a} | | |
| Mean (SD) | 140.0 (51.02) | 157.3 (33.87) |
| Median | 169.0 | 169.0 |
| Minimum, maximum | 14, 194 | 12, 192 |
| Mean daily dose (mg)^{b} | | |
| Mean (SD) | 49.0 (3.43) | 291.1 (23.84) |
| Median | 49.5 | 294.7 |
| Minimum, maximum | 36, 62 | 168, 337 |

Through Week 76 (i.e., End of Study)

| | | |
|---|---|---|
| Duration of dosing^{a} | | |
| Mean (SD) | 185.6 (93.01) | 206.6 (75.12) |
| Median | 190.0 | 194.0 |
| Minimum, maximum | 14, 351 | 12, 386 |
| Mean daily dose (mg)^{b} | (N = 44) | (N = 40) |
| Mean (SD) | 49.0 (3.47) | 290.3 (24.43) |
| Median | 49.6 | 295.4 |
| Minimum, maximum | 36, 62 | 168, 333 |

SD = standard deviation
^{a}Duration of dosing is the last dose date minus the first dose date + 1.
^{b}Mean daily dose is the total daily dose divided by the number of days dosed.

Sixty-three subjects completed at least 28 weeks of dosing during Part 2 and are counted as remaining in the study through Week 76. Twenty-nine subjects prematurely discontinued during the double-blind treatment period through Week 76.

Forty-six of the 97 subjects (47%) had at least 1 AE during the placebo washout period. Seven subjects (7%) had 12 AEs considered by the Investigator to be possibly or probably related to study drug. A total of 4 subjects had AEs considered severe in intensity.

Three subjects died due to TEAEs during the placebo washout period; all 3 deaths were considered to be related to ALS disease progression. Five subjects had SAEs, none of which were considered to be treatment-related. One subject who was assigned to the 300 mg group in Part 1 had an AE that began during the placebo washout period (neutropenia) that resulted in discontinuation of study drug during the double-blind treatment period. A summary of AEs during the placebo washout period is presented in TABLE 7.

TABLE 7
Summary of Treatment-Emergent Adverse Events -
Placebo Washout Period (Safety Population)

| | Study Drug During Part 1 of CL201 | | | | |
|---|---|---|---|---|---|
| | | dexpramipexole (total daily dose) | | | All |
| Treatment-Emergent Adverse | Placebo | 50 mg | 150 mg | 300 mg | Subjects |
| Events (TEAEs) | (N = 26) | (N = 22) | (N = 25) | (N = 24) | (N = 97) |
| ≧1 TEAE | 13 (50%) | 9 (41%) | 13 (52%) | 11 (46%) | 46 (47%) |
| Number of TEAEs | 34 | 18 | 29 | 46 | 127 |
| ≧1 treatment-related^{a} TEAE | 1 (4%) | 2 (9%) | 2 (8%) | 2 (8%) | 7 (7%) |
| Number of treatment-related TEAEs | 2 | 2 | 5 | 3 | 12 |
| ≧1 severe TEAE | 1 (4%) | 2 (9%) | 0 | 1 (4%) | 4 (4%) |
| Number of severe TEAEs | 1 | 2 | 0 | 1 | 4 |
| ≧1 TEAE with an outcome of death | 1 (4%) | 2 (9%) | 0 | 0 | 3 (3%) |
| ≧1 serious TEAE (including death) | 2 (8%) | 2 (9%) | 0 | 1 (4%) | 5 (5%) |
| Number of serious TEAEs | 3 | 2 | 0 | 1 | 6 |
| ≧1 serious treatment-related^{a} TEAE | 0 | 0 | 0 | 0 | 0 |
| Subjects with a TEAE with action | 0 | 0 | 0 | 1 (4%)^{b} | 1 (1%) |
| taken of study drug discontinued | | | | | |

^{a}Treatment-related are adverse events with a possible, probable, or unknown relationship to study medication.
^{b}Subject did not discontinue study drug until double-blind treatment period.

Eighty-seven of the 92 randomized subjects (95%) had at least one AE through Week 28 (TABLE 8). The overall incidence of AEs was similar in the 2 treatment groups through Week 28 (96% in the 50 mg group and 93% in the 300 mg group). The overall incidence of AEs considered by the Investigator to be possibly or probably related to study drug through Week 28 was 31% in the 50 mg group and 41% in the 300 mg group. A total of 18 subjects had AEs considered severe in intensity. Eight subjects had TEAEs with an outcome of death through Week 28. Sixteen subjects, 11 in the 50 mg group and 5 in the 300 mg group, had SAEs, 2 of whom had events considered to be treatment-related. One subject in the 50 mg group had AEs that led to premature discontinuation. The incidence of AEs through the end of the study was generally similar to that through Week 28. Through the end of the study, a total of 11 subjects (7 in 50 mg group and 4 in 300 mg group) died and 5 subjects (2 in 50 mg group and 3 in 300 mg group) had study drug discontinued due to AEs. A summary of AEs during the double-blind treatment period is presented in TABLE 8.

TABLE 8
Summary of Treatment-Emergent Adverse
Events (Safety Population)

| | 50 mg | 300 mg |
|---|---|---|
| Treatment-Emergent Adverse Events (TEAEs) | (N = 48) | (N = 44) |
| Through Week 28 | | |
| Subjects with ≧ 1TEAE | 46 (96%) | 41 (93%) |
| Number of TEAEs | 277 | 303 |
| ≧1 treatment-related^{a} TEAE | 15 (31%) | 18 (41%) |
| Number of treatment-related TEAEs | 35 | 46 |
| ≧1 severe TEAE | 12 (25%) | 6 (14%) |
| Number of severe TEAEs | 17 | 12 |
| ≧1 TEAE with an outcome of death^{b} | 7 (15%) | 1 (2%) |
| ≧1 serious TEAE (including death) | 11 (23%) | 5 (11%) |
| Number of serious TEAEs | 15 | 9 |
| ≧1 serious treatment-related^{a} TEAE | 1 (2%) | 1 (2%) |
| Number of serious treatment-related TEAE | 1 | 1 |
| Subjects with a TEAE with action | 1 (2%) | 0 |
| taken of study drug discontinued | | |
| ≧1 TEAE | 47 (98%) | 41 (93%) |
| Number of TEAEs | 333 | 366 |
| ≧1 treatment-relateda TEAE | 16 (33%) | 18 (41%) |
| Number of treatment-related TEAEs | 39 | 61 |
| ≧1 severe TEAE | 13 (27%) | 13 (30%) |
| Number of severe TEAEs | 20 | 23 |
| ≧1 TEAE with an outcome of deathb | 7 (15%) | 4 (9%) |
| ≧1 serious TEAE (including death) | 14 (29%) | 11 (25%) |
| Number of serious TEAEs | 18 | 17 |
| ≧1 serious treatment-relateda TEAE | 1 (2%) | 2 (5%) |
| Number of serious treatment-related TEAEs | 1 | 2 |
| Subjects with a TEAE with action | 2 (4%) | 3 (7%) |
| taken of study drug discontinued | | |

^{a}Treatment-related are adverse events with a possible, probable, or unknown relationship to study medication.
^{b}Excludes deaths in subjects who discontinued the study for reasons other than fatal adverse event.

Forty-six subjects (47%) reported TEAEs during the placebo washout period. A summary of frequently reported (≧5% of subjects in any Part 1 treatment group) AEs during the placebo washout period is presented by SOC and preferred term in TABLE 9.

TABLE 9
Number of Subjects Reporting Common (at least 5% of Subjects in
Any Treatment Group) Treatment-Emergent Adverse Events by SOC and
Preferred Term - Placebo Washout Period (Safety Population)

| | Study Drug During Part 1 of CL201 | | | | |
|---|---|---|---|---|---|
| | | dexpramipexole (total daily dose) | | | All |
| System Organ Class | Placebo | 50 mg | 150 mg | 300 mg | Subjects |
| Preferred Term | (N = 26) | (N = 22) | (N = 25) | (N = 24) | (N = 97) |
| Subjects with ≧1 TEAE | 13 (50%) | 9 (41%) | 13 (52%) | 11 (46%) | 46 (47%) |
| Gastrointestinal Disorders | 5 (19%) | 0 | 5 (20%) | 3 (13%) | 13 (13%) |
| Constipation | 2 (8%) | 0 | 1 (4%) | 3 (13%) | 6 (6%) |
| Diarrhoea | 0 | 0 | 2 (8%) | 1 (4%) | 3 (3%) |
| Nausea | 1 (4%) | 0 | 1 (4%) | 2 (8%) | 4 (4%) |
| General Disorders and | 1 (4%) | 2 (9%) | 1 (4%) | 3 (13%) | 7 (7%) |
| Administration Site Conditions | | | | | |
| Pyrexia | 0 | 1 (5%) | 0 | 2 (8%) | 3 (3%) |
| Injury, Poisoning and Procedural | 4 (15%) | 2 (9%) | 4 (16%) | 6 (25%) | 16 (16%) |
| Complications | | | | | |
| Fall | 3 (12%) | 2 (9%) | 3 (12%) | 3 (13%) | 11 (11%) |
| Musculoskeletal and Connective | 3 (12%) | 6 (27%) | 3 (12%) | 2 (8%) | 14 (14%) |
| Tissue Disorders | | | | | |
| Muscular weakness | 2 (8%) | 4 (18%) | 1 (4%) | 0 | 7 (7%) |
| Back pain | 0 | 2 (9%) | 0 | 1 (4%) | 3 (3%) |
| Psychiatric Disorders | 3 (12%) | 1 (5%) | 2 (8%) | 0 | 6 (6%) |
| Anxiety | 2 (8%) | 1 (5%) | 0 | 0 | 3 (3%) |
| Respiratory, Thoracic and | 2 (8%) | 1 (5%) | 2 (8%) | 1 (4%) | 6 (6%) |
| Mediastinal Disorders | | | | | |
| Cough | 2 (8%) | 0 | 2 (8%) | 0 | 4 (4%) |

TEAE = treatment-emergent adverse event

Overall, the most common (≧5% overall) TEAEs were fall (11%), muscular weakness (7%), and constipation (6%). Of note, of 7 subjects who reported muscular weakness during the placebo washout period, all but 1 had received placebo or 50 mg dexpramipexole during Part 1 of the study. Conversely, diarrhea and constipation were more common among subjects who had received higher doses of dexpramipexole during Part 1. A summary of AEs reported by ≧3% of subjects overall during the placebo washout period is presented by preferred term in descending order of frequency in TABLE 10.

TABLE 10
Number of Subjects Reporting Common (at least 3% of Subjects Overall)
Treatment-Emergent Adverse Events by Preferred Term in Decreasing
Frequency - Placebo Washout Period (Safety Population)

| | Study Drug During Part 1 of CL201 | | | | |
|---|---|---|---|---|---|
| | | dexpramipexole (total daily dose) | | | |
| | Placebo | 50 mg | 150 mg | 300 mg | All Subjects |
| Preferred Term | (N = 26) | (N = 22) | (N = 25) | (N = 24) | (N = 97) |
| Subjects with ≧1 TEAE | 13 (50%) | 9 (41%) | 13 (52%) | 11 (46%) | 46 (47%) |
| Fall | 3 (12%) | 2 (9%) | 3 (12%) | 3 (13%) | 11 (11%) |
| Muscular weakness | 2 (8%) | 4 (18%) | 1 (4%) | 0 | 7 (7%) |
| Constipation | 2 (8%) | 0 | 1 (4%) | 3 (13%) | 6 (6%) |
| Nausea | 1 (4%) | 0 | 1 (4%) | 2 (8%) | 4 (4%) |
| Cough | 2 (8%) | 0 | 2 (8%) | 0 | 4 (4%) |
| Back pain | 0 | 2 (9%) | 0 | 1 (4%) | 3 (3%) |
| Diarrhea | 0 | 0 | 2 (8%) | 1 (4%) | 3 (3%) |
| Muscle spasms | 0 | 1 (5%) | 1 (4%) | 1 (4%) | 3 (3%) |
| Pyrexia | 0 | 1 (5%) | 0 | 2 (8%) | 3 (3%) |
| Dyspnoea | 1 (4%) | 1 (5%) | 0 | 1 (4%) | 3 (3%) |
| Post lumbar | 1 (4%) | 0 | 1 (4%) | 1 (4%) | 3 (3%) |
| puncture syndrome | | | | | |
| Anxiety | 2 (8%) | 1 (5%) | 0 | 0 | 3 (3%) |

TEAE = treatment-emergent adverse event
Note:
All Investigator adverse event terms were coded using MedDRA dictionary Version 11.0.

The overall incidence of AEs was similar in the 50 mg group (96%) and the 300 mg group (93%) (TABLE 11). The incidence of specific AEs was generally similar in the treatment groups. Four AEs had at least a 10% difference in incidence between the 2 treatment groups. Dry mouth and insomnia occurred at a higher incidence in the 300 mg group (16% and 14%, respectively) than in the 50 mg group (2% and 0%, respectively), while muscular weakness and peripheral edema occurred at a higher incidence in the 50 mg group (27% and 15%, respectively) than in the 300 mg group (16% and 2%, respectively). A summary of frequently reported (≧5% of subjects in either treatment group) AEs during the double-blind treatment period through Week 28 is presented by SOC and preferred term in TABLE 11.

TABLE 11
Number of Subjects Reporting Common (at least 5% of Subjects in
Either Treatment Group) Treatment-Emergent Adverse Events by SOC
and Preferred Term-Double-Blind Treatment Period Through Week 28
(Safety Population)

| System Organ Class | 50 mg | 300 mg |
|---|---|---|
| Preferred Term | (N = 48) | (N = 44) |
| Subjects with ≧ 1treatment-emergent adverse event | 46 (96%) | 41 (93%) |
| Cardiac Disorders | 2 (4%) | 8 (18%) |
| Tachycardia | 1 (2%) | 4 (9%) |
| Gastrointestinal Disorders | 21 (44%) | 25 (57%) |
| Constipation | 8 (17%) | 11 (25%) |
| Salivary hypersecretion | 8 (17%) | 5 (11%) |
| Dysphagia | 5 (10%) | 4 (9%) |
| Dry mouth | 1 (2%) | 7 (16%) |
| Nausea | 4 (8%) | 3 (7%) |
| Vomiting | 3 (6%) | 0 |
| General Disorders and Administration | 14 (29%) | 12 (27%) |
| Site Conditions | | |
| Oedema peripheral | 7 (15%) | 1 (2%) |
| Fatigue | 3 (6%) | 2 (5%)^{a} |
| Infections and Infestations | 21 (44%) | 17 (39%) |
| Upper respiratory tract infection | 5 (10%) | 3 (7%) |
| Sinusitis | 2 (4%) | 5 (11%) |
| Urinary tract infection | 3 (6%) | 4 (9%) |
| Pneumonia | 3 (6%) | 1 (2%) |
| Injury, Poisoning and Procedural Complication | 15 (31%) | 12 (27%) |
| Fall | 11 (23%) | 11 (25%) |
| Metabolism and Nutrition Disorders | 6 (13%) | 8 (18%) |
| Dehydration | 1 (2%) | 3 (7%) |
| Hyperglycaemia | 0 | 3 (7%) |
| Musculoskeletal and Connective Tissue Disorders | 22 (46%) | 19 (43%) |
| Muscular weakness | 13 (27%) | 7 (16%) |
| Arthralgia | 2 (4%) | 3 (7%) |
| Musculoskeletal pain | 2 (4%) | 3 (7%) |
| Neck pain | 1 (2%) | 4 (9%) |
| Nervous System Disorders | 16 (33%) | 18 (41%) |
| Dysarthria | 4 (8%) | 5 (11%) |
| Headache | 3 (6%) | 6 (14%) |
| Muscle spasticity | 5 (10%) | 1 (2%) |
| Muscle contractions involuntary | 3 (6%) | 2 (5%)^{a} |
| Dizziness | 0 | 3 (7%) |
| Psychiatric Disorders | 9 (19%) | 14 (32%) |
| Depression | 6 (13%) | 5 (11%) |
| Anxiety | 2 (4%) | 5 (11%) |
| Insomnia | 0 | 6 (14%) |
| Respiratory, Thoracic and Mediastinal Disorders | 17 (35%) | 18 (41%) |
| Dyspnoea | 5 (10%) | 6 (14%) |
| Pharyngolaryngeal pain | 4 (8%) | 3 (7%) |
| Cough | 1 (2%) | 5 (11%) |
| Respiratory failure | 4 (8%) | 1 (2%) |
| Skin and Subcutaneous Tissue Disorders | 14 (29%) | 8 (18%) |
| Rash | 2 (4%) | 3 (7%) |
| Pruritus | 3 (6%) | 0 |

^{a}Incidence was 4.5%, which rounded to 5%.

Frequently reported AEs (≧10% of subjects overall) during the double-blind treatment period through Week 28 were fall (22 subjects, 24%), muscular weakness (20 subjects, 22%), constipation (19 subjects, 21%), salivary hypersecretion (13 subjects, 14%), depression (11 subjects, 12%), and dyspnoea (11 subjects, 12%). A summary of AEs reported by ≧5% of subjects overall (≧5 subjects) in the double-blind treatment period through Week 28 is presented by preferred term in descending order of frequency in TABLE 12.

TABLE 12
Number of Subjects Reporting Common (at least 5% of Subjects Overall)
Treatment-Emergent Adverse Events by Preferred Term in Decreasing
Frequency-Double-Blind Treatment Period Through Week 28
(Safety Population)

| | 50 mg | 300 mg |
|---|---|---|
| Preferred Term | (N = 48) | N = 44) |
| Subjects with ≧ 1 treatment-emergent adverse event | 46 (96%) | 41 (93%) |
| Fall | 11 (23%) | 11 (25%) |
| Muscular weakness | 13 (27%) | 7 (16%) |
| Constipation | 8 (17%) | 11 (25%) |
| Salivary hypersecretion | 8 (17%) | 5 (11%) |
| Depression | 6 (13%) | 5 (11%) |
| Dyspnoea | 5 (10%) | 6 (14%) |
| Dysarthria | 4 (8%) | 5 (11%) |
| Dysphagia | 5 (10%) | 4 (9%) |
| Headache | 3 (6%) | 6 (14%) |
| Dry mouth | 1 (2%) | 7 (16%) |
| Oedema peripheral | 7 (15%) | 1 (2%) |
| Upper respiratory tract infection | 5 (10%) | 3 (7%) |
| Anxiety | 2 (4%) | 5 (11%) |
| Nausea | 4 (8%) | 3 (7%) |
| Pharyngolaryngeal pain | 4 (8%) | 3 (7%) |
| Sinusitis | 2 (4%) | 5 (11%) |
| Urinary tract infection | 3 (6%) | 4 (9%) |
| Cough | 1 (2%) | 5 (11%) |
| Insomnia | 0 | 6 (14%) |
| Muscle spasticity | 5 (10%) | 1 (2%) |
| Arthralgia | 2 (4%) | 3 (7%) |
| Fatigue | 3 (6%) | 2 (5%) |
| Muscle contractions involuntary | 3 (6%) | 2 (5%) |
| Musculoskeletal pain | 2 (4%) | 3 (7%) |
| Neck pain | 1 (2%) | 4 (9%) |
| Rash | 2 (4%) | 3 (7%) |
| Respiratory failure | 4 (8%) | 1 (2%) |
| Tachycardia | 1 (2%) | 4 (9%) |

The overall incidence of TEAEs during the double-blind treatment period through the end of the study was similar in the 50 mg group (98%) and the 300 mg group (93%). In addition, the AE profile through the end of the study (TABLE 12) was similar to that through Week 28.

Seven (7%) subjects (1 placebo; 2 each in 50 mg, 150 mg, and 300 mg Part 1 groups) reported treatment-related AEs during the placebo washout period. Two subjects each reported constipation (1 each in placebo and 150 mg groups) and headache (1 each in 50 mg and 150 mg groups). All other treatment-related AEs were reported by 1 subject each. Treatment-emergent, treatment-related AEs reported by 1 subject during the placebo washout period included fall (placebo); petechiae (50 mg); dry mouth, nausea, vomiting, and pruritus (150 mg); and neutropenia (300 mg). The subject with neutropenia had the event reported at baseline of the placebo washout period, which was the Part 1, Week 12 visit.

Of the 87 subjects who reported TEAEs through Week 28 of the double-blind treatment period, 33 had events that were considered to be possibly or probably treatment-related. The overall incidence of treatment-related AEs was 31% in the 50 mg group and 41% in the 300 mg group. Treatment-related AEs were most commonly associated with Gastrointestinal Disorders and Nervous System Disorders. The most common treatment-related AEs overall included constipation (5 subjects, 5%), headache (5 subjects, 5%), and dry mouth (4 subjects, 4%). Gastrointestinal AEs were more common in the 300 mg group than in the 50 mg group.

The incidence of treatment-emergent, treatment-related AEs through the end of the study was similar to that through Week 28.

As assessed by the Investigator, 1 or more TEAEs related to ALS were reported by 24 subjects (25%) during the placebo washout period (TABLE 13). The most common (≧5% overall) ALS-related AEs overall included fall (10%) and muscular weakness (6%). A summary of treatment-emergent ALS-related AEs reported in at least 2 subjects overall during the placebo washout period is presented in TABLE 13.

TABLE 13
Treatment-Emergent ALS-Related Adverse Events Reported in at Least Two
Subjects Overall During the Placebo Washout Period (Safety Population)

| | Study Drug During Part 1 of CL201 | | | | |
|---|---|---|---|---|---|
| | | dexpramipexole (total daily dose) | | | All |
| System Organ Class | Placebo | 50 mg | 150 mg | 300 mg | Subjects |
| Preferred Term | (N = 26) | (N = 22) | (N = 25) | (N = 24) | (N = 97) |
| Subjects with ≧1 ALS-related TEAE | 5 (19%) | 7 (32%) | 7 (28%) | 5 (21%) | 24 (25%) |
| General Disorders and | 1 (4%) | 2 (9%) | 0 | 0 | 3 (3%) |
| Administration Site Conditions | | | | | |
| Disease progression | 1 (4%) | 1 (5%) | 0 | 0 | 2 (2%) |
| Injury, Poisoning and Procedural | 3 (12%) | 2 (9%) | 3 (12%) | 2 (8%) | 10 (10%) |
| Complications | | | | | |
| Fall | 3 (12%) | 2 (9%) | 3 (12%) | 2 (8%) | 10 (10%) |
| Musculoskeletal and Connective | 1 (4%) | 4 (18%) | 2 (8%) | 1 (4%) | 8 (8%) |
| Tissue Disorders | | | | | |
| Muscular weakness | 1 (4%) | 4 (18%) | 1 (4%) | 0 | 6 (6%) |
| Muscle spasms | 0 | 1 (5%) | 1 (4%) | 1 (4%) | 3 (3%) |
| Respiratory, Thoracic and | 1 (4%) | 1 (5%) | 1 (4%) | 1 (4%) | 4 (4%) |
| Mediastinal Disorders | | | | | |
| Dyspnoea | 1 (4%) | 1 (5%) | 0 | 1 (4%) | 3 (3%) |

ALS = amyotrophic lateral sclerosis; TEAE = treatment-emergent adverse event

As assessed by the Investigator, the majority of AEs in both treatment groups were related to ALS during the double-blind treatment period through Week 28. One or more TEAEs related to ALS were reported by 79% of the 50 mg group and 77% of the 300 mg group (TABLE 14). The most common ALS-related AEs overall included fall (20 subjects, 22%), muscular weakness (19 subjects, 21%), and salivary hypersecretion (13 subjects, 14%). A summary of treatment-emergent ALS-related AEs reported in at least 2 subjects overall during the double-blind treatment period through Week 28 is presented in TABLE 14.

TABLE 14
Treatment-Emergent ALS-Related Adverse Events Reported in at Least
Two Subjects Overall During the Double-Blind Treatment Period
Through Week 28 (Safety Population)

| System Organ Class | 50 mg | 300 mg |
|---|---|---|
| Preferred Term | (N = 48) | (N = 44) |
| Subjects with ≧ 1 ALS-related TEAE | 38 (79%) | 34 (77%) |
| Gastrointestinal Disorders | 16 (33%) | 11 (25%) |
| Salivary hypersecretion | 8 (17%) | 5 (11%) |
| Constipation | 4 (8%) | 4 (9%) |
| Dysphagia | 5 (10%) | 3 (7%) |
| General Disorders and Administration | 8 (17%) | 6 (14%) |
| Site Conditions | | |
| Fatigue | 3 (6%) | 2 (5%) |
| Disease progression | 2 (4%) | 2 (5%) |
| Oedema peripheral | 3 (6%) | 1 (2%) |
| Injury, Poisoning and Procedural Complications | 10 (21%) | 10 (23%) |
| Fall | 10 (21%) | 10 (23%) |
| Contusion | 1 (2%) | 1 (2%) |
| Investigations | 3 (6%) | 5 (11%) |
| Vital capacity decreased | 2 (4%) | 1 (2%) |
| Weight decreased | 0 | 2 (5%) |
| Metabolism and Nutrition Disorders | 1 (2%) | 4 (9%) |
| Dehydration | 0 | 2 (5%) |
| Musculoskeletal and Connective Tissue Disorders | 19 (40%) | 14 (32%) |
| Muscular weakness | 12 (25%) | 7 (16%) |
| Musculoskeletal pain | 1 (2%) | 3 (7%) |
| Muscle twitching | 1 (2%) | 2 (5%) |
| Arthralgia | 1 (2%) | 1 (2%) |
| Myalgia | 2 (4%) | 0 |
| Neck pain | 1 (2%) | 1 (2%) |
| Pain in extremity | 2 (4%) | 0 |
| Nervous System Disorders | 11 (23%) | 10 (23%) |
| Dysarthria | 4 (8%) | 5 (11%) |
| Muscle spasticity | 5 (10%) | 1 (2%) |
| Muscle contractions involuntary | 3 (6%) | 2 (5%) |
| Dysphasia | 1 (2%) | 1 (2%) |
| Psychiatric Disorders | 2 (4%) | 1 (2%) |
| Affect liability | 2 (4%) | 0 |
| Depression | 1 (2%) | 1 (2%) |
| Respiratory, Thoracic and Mediastinal Disorders | 11 (23%) | 11 (25%) |
| Dyspnoea | 4 (8%) | 5 (11%) |
| Respiratory failure | 4 (8%) | 0 |
| Increased upper airway secretion | 1 (2%) | 2 (5%) |
| Choking | 2 (4%) | 0 |
| Dyspnoea exertional | 0 | 2 (5%) |

ALS = amyotrophic lateral sclerosis; TEAE = treatment-emergent adverse event

During the placebo washout period, the majority of AEs in each treatment group were considered to be mild or moderate in intensity. Severe AEs were reported for 4 (4%) subjects (disease progression and dyspnoea in 2 subjects each), none of which were considered to be related to study drug.

During the double-blind treatment period through Week 28, the majority of AEs in both treatment groups were considered by the Investigator to be mild or moderate in intensity. Severe AEs reported for more than 1 subject included respiratory failure (5 subjects) and dyspnoea (2 subjects). One or more severe AEs were reported for 12 (25%) subjects in the 50 mg group (acute myocardial infarction; ileus; fatigue; disease progression; sudden death; pneumonia bacterial; rib fracture; hypernatraemia; muscular weakness; muscle contractions involuntary; dyspnoea; respiratory failure [4 subjects]; respiratory distress; pulmonary embolism) and 6 (14%) subjects in the 300 mg group (neutropenia; dry mouth; cholecystitis acute; pneumonia; fall; concussion; subdural haematoma; vital capacity decreased; dizziness; dyspnoea; pharyngolaryngeal pain; respiratory failure). During the double-blind treatment period through the end of the study, the majority of AEs in both treatment groups were considered to be mild or moderate in intensity. Severe AEs were reported for 13 subjects in each treatment group.

During the double-blind treatment period, all (100%) subjects in both treatment groups who did not use riluzole at baseline had TEAEs (TABLE 15); among subjects who used riluzole at baseline, 96% of riluzole users in the 50 mg group and 90% of riluzole users in the 300 mg group had one or more AEs (TABLE 15). The incidence of common TEAEs was compared in each treatment group among subjects who were and were not using riluzole at baseline. Adverse events that had at least a 10% greater incidence among subjects who did or did not use riluzole were noted. In the 50 mg group, subjects who were not taking riluzole had a higher incidence of salivary hypersecretion (22% vs. 12%) and dysphagia (17% vs. 4%) than subjects who were taking riluzole. In the 300 mg group, subjects who were not taking riluzole had a higher incidence of dyspnoea (27% vs. 7%), headache (27% vs. 2%), dry mouth (27% vs. 10%), and upper respiratory tract infection (13% vs. 3%). Conversely, subjects in the 300 mg group who were taking concomitant riluzole had a higher incidence of constipation (31% vs. 13%), nausea (10% vs. 0%), and sinusitis (17% vs. 0%) than subjects who were not taking riluzole. The incidence of TEAEs through the end of the study for subjects who did and did not use riluzole at baseline (TABLE 18) was similar to that through Week 28.

TABLE 15
Summary of Frequent Adverse Events by Baseline Riluzole Use During
the Double-Blind Treatment Period Through Week 28

| | 50 mg | | 300 mg | |
|---|---|---|---|---|
| | (N = 48) | | (N = 44) | |
| | Riluzole Use | No Riluzole Use | Riluzole Use | No Riluzole Use |
| Preferred Term | (N = 25) | (N = 23) | (N = 29) | (N = 15) |
| Subjects with ≧ 1 TEAE | 24 (96%) | 23 (100%) | 26 (90%) | 15 (100%) |
| Fall | 5 (20%) | 6 (26%) | 7 (24%) | 4 (27%) |
| Muscular weakness | 6 (24%) | 7 (30%) | 4 (14%) | 3 (20%) |
| Constipation | 4 (16%) | 4 (17%) | 9 (31%) | 2 (13%) |
| Salivary hypersecretion | 3 (12%) | 5 (22%) | 3 (10%) | 2 (13%) |
| Depression | 2 (8%) | 4 (17%) | 4 (14%) | 1 (7%) |
| Dyspnoea | 2 (8%) | 3 (13%) | 2 (7%) | 4 (27%) |
| Dysarthria | 3 (12%) | 1 (4%) | 3 (10%) | 2 (13%) |
| Dysphagia | 1 (4%) | 4 (17%) | 2 (7%) | 2 (13%) |
| Headache | 2 (8%) | 1 (4%) | 2 (7%) | 4 (27%) |
| Dry mouth | 1 (4%) | 0 | 3 (10%) | 4 (27%) |
| Oedema peripheral | 3 (12%) | 4 (17%) | 1 (3%) | 0 |
| Upper respiratory tract infection | 3 (12%) | 2 (9%) | 1 (3%) | 2 (13%) |
| Anxiety | 1 (4%) | 1 (4%) | 4 (14%) | 1 (7%) |
| Nausea | 2 (8%) | 2 (9%) | 3 (10%) | 0 |
| Pharyngolaryngeal pain | 3 (12%) | 1 (4%) | 2 (7%) | 1 (7%) |
| Sinusitis | 1 (4%) | 1 (4%) | 5 (17%) | 0 |
| Urinary tract infection | 2 (8%) | 1 (4%) | 3 (10%) | 1 (7%) |

Three subjects had TEAEs with an outcome of death during the placebo washout period. All 3 deaths were ALS-related; 2 deaths were due to disease progression (1 in placebo, 1 in 50 mg) and 1 death was due to dyspnoea (50 mg). Eight subjects (7 in 50 mg, 1 in 300 mg) had TEAEs with an outcome of death during the double-blind treatment period through Week 28; 5 deaths were due to respiratory failure, with 1 of these subjects also having pneumonia, and 1 death each was due to ileus, disease progression, and sudden death. Three additional subjects (300 mg) died through the end of the study due to TEAEs; 1 death each was due to disease progression, traumatic intracranial hemorrhage, and respiratory failure. These deaths exclude subjects who died after discontinuing the study for reasons other than fatal AE.

During the placebo washout period, 1 subject who received 300 mg during Part 1 required a tracheostomy. Three subjects (1 subject who received placebo during Part 1 and 2 subjects who received 50 mg during Part 1) died during the placebo washout period. None of the subjects required tracheostomy through Week 28 of the double-blind treatment period. In the double-blind treatment period through Week 28, 9 (19%) subjects in the 50 mg group and 3 (7%) subjects in the 300 mg group died.

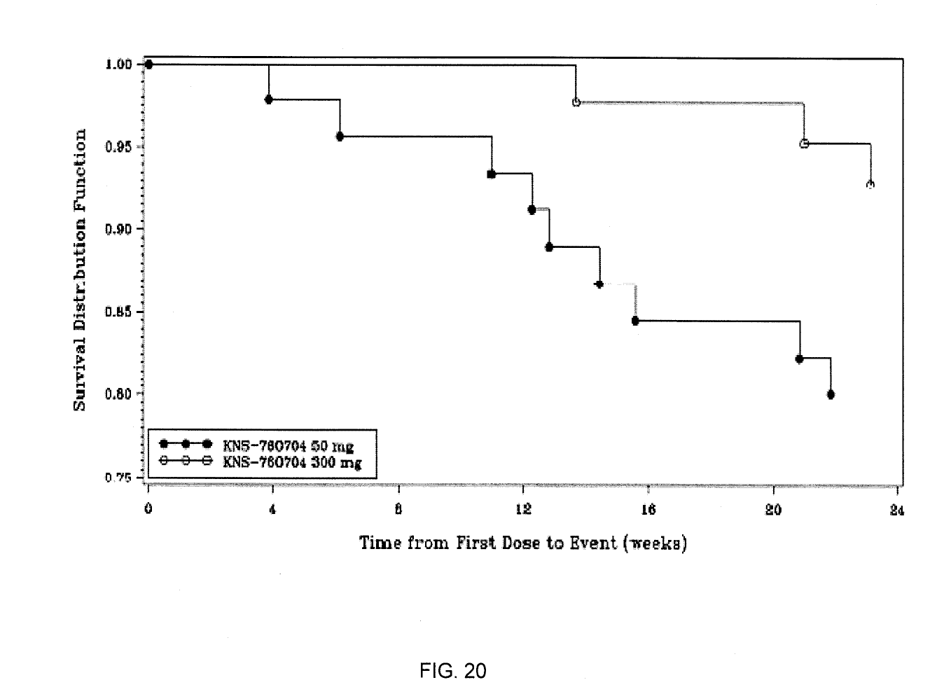

Of the 12 subjects who died during the double-blind treatment period through Week 28, 2 subjects in the 50 mg group and 1 in the 300 mg group died following discontinuation from the study. These 3 subjects had previously withdrawn consent due to their inability to travel to required clinic visits and were not on active study drug at the time of death. The reduction in the hazard ratio for time to tracheostomy or death for the 300 mg group relative to the 50 mg group was 68%. Based on a log rank test, the difference between the 2 treatment groups in time to tracheostomy or death approached statistical significance (p=0.071). FIG. 20 provides a graphic presentation of the Kaplan-Meier estimates for the time to tracheostomy or death through Week 28.

Five subjects had serious TEAEs during the placebo washout period, including the 3 subjects with fatal events (TABLE 16). Four of the 5 subjects had SAEs that were considered by the Investigator to be related to ALS; the other subject had 2 serious events (urethral obstruction and urinary retention) that were not ALS-related. None of the SAEs were considered by the Investigator to be related to study drug. A summary of SAEs during the placebo washout period is presented in TABLE 16.

TABLE 16
Summary of Treatment-Emergent Serious Adverse Events
During the Placebo Washout Period (Safety Population)

| | Study Drug During Part 1 of CL201 | | | | |
|---|---|---|---|---|---|
| | | dexpramipexole (total daily dose) | | | All |
| System Organ Class | Placebo | 50 mg | 150 mg | 300 mg | Subjects |
| Preferred Term | (N = 26) | (N = 22) | (N = 25) | (N = 24) | (N = 97) |
| Subjects with ≧1 serious TEAE | 2 (8%) | 2 (9%) | 0 | 1 (4%) | 5 (5%) |
| General Disorders and | 1 (4%) | 1 (5%) | 0 | 0 | 2 (2%) |
| Administration Site Conditions | | | | | |
| Disease progression | 1 (4%)^{a} | 1 (5%)^{a} | 0 | 0 | 2 (2%) |
| Renal and Urinary Disorders | 1 (4%)^{b} | 0 | 0 | 0 | 1 (1%) |
| Urethral obstruction | 1 (4%) | 0 | 0 | 0 | 1 (1%) |
| Urinary retention | 1 (4%) | 0 | 0 | 0 | 1 (1%) |
| Respiratory, Thoracic and | 0 | 1 (5%) | 0 | 1 (4%) | 2 (2%) |
| Mediastinal Disorders | | | | | |
| Dyspnoea | 0 | 1 (5%)^{a} | 0 | 1 (4%) | 2 (2%) |

^{a}Fatal.
^{b}One subject had 2 SAEs, urethral obstruction and urinary retention.

Sixteen subjects, 11 in the 50 mg group and 5 in the 300 mg group, had serious TEAEs during the double-blind treatment period through Week 28, including the 8 subjects with fatal events (TABLE 17). The most common SAEs were respiratory failure (5 subjects) and pneumonia (2 subjects); all other SAEs were reported by 1 subject each. Six of these subjects had SAEs that were considered by the Investigator to be related to ALS (respiratory failure [4 subjects], disease progression, dyspnoea, pneumonia bacterial, and pneumonia aspiration). Twenty-five subjects, 14 in the 50 mg group and 11 in the 300 mg group, had serious TEAEs during the double-blind treatment period through the end of the study, including 11 subjects with fatal events. The most common SAEs were respiratory failure (6 subjects), pneumonia (4 subjects), disease progression (2 subjects), and pneumonia aspiration (2 subjects); all other SAEs were reported in 1 subject each. A summary of SAEs during the double-blind treatment period through Week 28 is presented in TABLE 17.

TABLE 17
Summary of Treatment-Emergent Serious Adverse Events During the
Double-Blind Treatment Period (Safety Population)

| System Organ Class | 50 mg | 300 mg |
|---|---|---|
| Preferred Term | (N = 48) | (N = 44) |
| Subjects with ≧ 1 serious TEAE | 11 (23%) | 5 (11%) |
| Blood and Lymphatic System Disorders | 0 | 1 (2%) |
| Neutropenia | 0 | 1 (2%) |
| Cardiac Disorders | 1 (2%) | 1 (2%) |
| Acute myocardial infarction | 1 (2%) | 0 |
| Atrial fibrillation | 0 | 1 (2%) |
| Cardiac failure congestive | 1 (2%) | 0 |
| Gastrointestinal Disorders | 1 (2%) | 0 |
| Ileus | 1 (2%) | 0 |
| General Disorders and Administration | 2 (4%) | 0 |
| Site Conditions | | |
| Disease progression | 1 (2%) | 0 |
| Sudden death | 1 (2%) | 0 |
| Hepatobiliary Disorders | 0 | 1 (2%) |
| Cholecystitis acute | 0 | 1 (2%) |
| Infections and Infestations | 3 (6%) | 1 (2%) |
| Pneumonia | 1 (2%) | 1 (2%) |
| Pneumonia bacterial | 1 (2%) | 0 |
| Viral infection | 1 (2%) | 0 |
| Injury, Poisoning and Procedural Complications | 0 | 1 (2%) |
| Concussion | 0 | 1 (2%) |
| Fall | 0 | 1 (2%) |
| Subdural haematoma | 0 | 1 (2%) |
| Respiratory, Thoracic and Mediastinal Disorders | 5 (10%) | 2 (5%) |
| Respiratory failure | 4 (8%) | 1 (2%) |
| Dyspnoea | 1 (2%) | 0 |
| Pneumonia aspiration | 0 | 1 (2%) |
| Pulmonary embolism | 1 (2%) | 0 |
| Respiratory distress | 1 (2%) | 0 |

One subject had an AE that was reported at baseline of the placebo washout period (Part 1, Week 12 visit) that led to premature discontinuation during the double-blind treatment period and developed mild neutropenia on Day −17 of Part 2, with a neutrophil count of 1.18×10³/μL. The Investigator considered the event to be probably related to study drug and study drug was temporarily discontinued. The subject's neutrophil count again fell to 1200 U/L and study drug was discontinued on Day 71 of Part 2.

Three subjects had TEAEs with an outcome of death during the placebo washout period. A total of 12 subjects had TEAEs with an outcome of death during the double-blind treatment period through the end of the study. Following termination from the trial, subjects were to be followed for living status every 3 months, through the closure of the study. This information was obtained by a health care professional via a telephone or email contact with the subject or caregiver. Six additional subjects died following discontinuation from the study: Excluding subject deaths, 2 subjects had SAEs during the placebo washout period and 17 subjects had SAEs during the double-blind treatment period study. Three of these subjects, 1 in the 50 mg group and 2 in the 300 mg group, had SAEs during the double-blind treatment period and subsequently had TEAEs with an outcome of deathDuring the placebo washout period, minor mean increases in neutrophil count were observed in all Part 1 treatment groups, except the 50 mg group; minor median increases were observed in all Part 1 treatment groups (TABLE 18).

TABLE 18
Mean Change from Baseline to Week 4 in Neutrophil Count (×
10³/μL) Values - Placebo Washout Period (Safety Population)

| | Study Drug During Part 1 of CL201 | | | | |
|---|---|---|---|---|---|
| | | dexpramipexole (total daily dose) | | | |
| | Placebo | 50 mg | 150 mg | 300 mg | All Subjects |
| Neutrophil Count (×10³/μL) | (N = 26) | (N = 22) | (N = 25) | (N = 24) | (N = 97) |
| Baseline (Part 1: Week 12) | (N = 24) | (N = 21) | (N = 23) | (N = 24) | (N = 92) |
| Mean (SD) | 4.268 (2.1739) | 4.539 (1.3112) | 3.931 (1.4130) | 3.905 (1.9377) | 4.151 (1.7523) |
| Part 2: Week 4 | (N = 22)^{a} | (N = 18)^{a} | (N = 23)^{a} | (N = 22) | (N = 85)^{a} |
| Mean (SD) | 4.498 (1.7736) | 4.470 (1.3436) | 4.753 (1.5327) | 4.112 (1.1979) | 4.466 (1.4839) |
| Mean Δ (SD) | 0.215 (1.5225) | −0.022 (0.7157) | 0.841 (1.1854) | 0.130 (1.3502) | 0.312 (1.2723) |

SD = standard deviation
^{a}Only subjects with both baseline and post-baseline values were summarized for change from baseline.

The overall incidence of AEs was similar in the 50 mg (96%) and 300 mg (93%) treatment groups during the double-blind treatment period. Frequently reported AEs (≧10% of subjects overall) during the double-blind treatment period through Week 28 were fall (24%), muscular weakness (22%), constipation (21%), salivary hypersecretion (14%), depression (12%), and dyspnoea (12%). The incidence of specific AEs was generally similar in the 2 treatment groups. Dry mouth and insomnia occurred at a higher incidence in the 300 mg group (16% and 14%, respectively) than in the 50 mg group (2% and 0%, respectively), while muscular weakness and peripheral edema occurred at a higher incidence in the 50 mg group (27% and 15%, respectively) than in the 300 mg group (16% and 2%, respectively).

The overall incidence of AEs considered by the Investigator to be possibly or probably related to study drug was 31% in the 50 mg group and 41% in the 300 mg group. The most common treatment-related AEs overall included constipation (5%), headache (5%), and dry mouth (4%). As assessed by the Investigator, the majority of AEs in both treatment groups were related to ALS (50 mg: 85%; 300 mg: 80%). The incidence of specific ALS-related AEs was generally similar in the 2 treatment groups. The most common ALS-related AEs overall included fall (24%), muscular weakness (23%), and salivary hypersecretion (17%).

During the double-blind treatment period, the majority of AEs in both treatment groups were considered by the Investigator to be mild or moderate in intensity. A total of 18 subjects, 12 in the 50 mg group and 6 in the 300 mg group, had AEs considered severe in intensity; respiratory failure, reported in a total of 5 subjects, was the most common severe event.

A total of 12 subjects (7 in 50 mg group, 5 in 300 mg group) had TEAEs with an outcome of death during the double-blind treatment period through the end of the study. In 8 of the 12 subjects, death was ALS-related. In all but 1 subject, death was considered to be unrelated or unlikely related to study drug. In 1 subject, the AE of sudden death was considered possibly related to study drug. Six additional subjects died following discontinuation from the study. All 6 subjects had withdrawn from the study due to inability to travel and/or declining functional status.

Sixteen subjects, 11 in the 50 mg group and 5 in the 300 mg group, had SAEs (including the 8 subjects with fatal events) though Week 28, 2 of which were considered to be possibly related to study drug (sudden death and neutropenia). Nine additional subjects (3 in the 50 mg group and 6 in the 300 mg group) had SAEs through the end of the study, one of which (pancreatitis in the 300 mg group) was considered to be possibly related to study drug. One subject (50 mg) discontinued treatment during the double-blind treatment period due to the AE of respiratory failure. Four additional subjects (1 in 50 mg group, 3 in 300 mg group) had AEs that led to premature discontinuation through the end of the study.

During the double-blind treatment period, mean changes from baseline to Week 28 for hematology and chemistry parameters were generally small in both of the treatment groups and not considered clinically meaningful. Only 1 subject (300 mg) had a potentially clinically significant hematology parameter, a hemoglobin value of 7.8 g/dL, which returned to near normal on Day 62 (11.0 g/dL). In addition, 1 subject (300 mg) had neutropenia (1.18×10³/μL) that was noted at baseline of the placebo washout period, continued in the double-blind period, and subsequently led to discontinuation of study drug. [3 subjects with neutropenia that began in Part 2]

Eight subjects, 4 in each treatment group, had serum chemistry abnormalities that met pre-specified criteria for potential clinical significance during the double-blind treatment period; 3 subjects had elevations in ALT (>3×ULN), 2 subjects each had elevations in glucose (>250 mg/dL) and alkaline phosphatase (>1.5×ULN), 1 subject each had an elevation in sodium (>157 mEq/L) and AST (3×ULN), and 1 subject each had a decrease in calcium (<7 mg/dL) and potassium (<2.5 mEq/L). Sixteen subjects had elevated AST or ALT values (>1.5×ULN) and 3 subjects had elevated AST and/or ALT (>3×ULN). Five of the 16 subjects with elevations in liver function test values had values that were considered clinically significant.

During the double-blind treatment period, minor mean changes from baseline in vital sign parameters were observed in both treatment groups. No clinically meaningful differences were observed in mean change from baseline to Week 28 or the endpoint of Part 2 in systolic blood pressure, diastolic blood pressure, respiratory rate, heart rate, or temperature. The incidence of blood pressure and pulse abnormalities that met pre-specified criteria for potential clinical significance was low. The most common potentially clinically significant change was for decrease in weight>7% from baseline (30 subjects).

Minor mean changes from baseline in ECG parameters were observed in both treatment groups during the double-blind treatment period, none of which were considered to be clinically meaningful. No differences were observed between the treatment groups in the incidence of post-baseline ECG abnormalities that met pre-specified criteria for potential clinical significance. The most common potentially clinically significant ECG abnormality was prolonged QTcB, reported for a total of 9 subjects (6 in 50 mg group and 3 in 300 mg group). Of the 9 subjects with a prolonged QTcB, 2 subjects, 1 in each treatment group, also had a prolonged QTcF. One subject (300 mg) had QTcB and QTcF intervals>500 msec.

The incidence of QTcB intervals that met specified threshold values (>450 msec, >480 msec, >500 msec) was generally similar in the 2 treatment groups. The incidence of ECGs with QTcB that increased>30 msec from baseline at any post-baseline visit was higher in the 300 mg group (30%) than in the 50 mg group (17%). One subject (300 mg) had an increase from baseline>60 msec in QTcB interval.

The incidence of QTcF intervals that met specified threshold values was low in both treatment groups. Two subjects in the 50 mg group and 3 subjects in the 300 mg group had a QTcF interval>450 msec at any post-baseline visit. Four subjects in the 50 mg group and 5 subjects in the 300 mg group had an increase from baseline in QTcF>30 msec. No subjects had an increase from baseline>60 msec.

Dexpramipexole may be a useful neuroprotective agent in the treatment of chronic and acute neurodegenerative disorders, including ALS. This was the first clinical study of dexpramipexole in subjects with ALS. Eligible subjects were ≧24 months from ALS symptom onset and met the clinically possible, clinically probable—laboratory-supported, clinically probable, or clinically definite El Escorial criteria. Part 1 of the current study evaluated the safety and tolerability of 3 dose levels of dexpramipexole (50 mg, 150 mg, and 300 mg given as 25 mg Q12H, 75 mg Q12H, and 150 mg Q12H, respectively) over 12 weeks of treatment in subjects with ALS. Subjects who completed Part 1 were eligible to enroll in Part 2 of the study. At the beginning of Part 2, all subjects participated in a single-blind, 4-week placebo washout and were observed for withdrawal effects. Following completion of the placebo washout period, subjects were re-randomized in a double-blind manner to low-dose (50 mg, administered as 25 mg Q12H) or high-dose (300 mg, administered as 150 mg Q12H) dexpramipexole to receive treatment for up to 76 weeks. Dexpramipexole was safe and well tolerated in ALS subjects over 24 weeks of active treatment at total daily doses of 50 mg and 300 mg. The majority of deaths (17/21) were considered to be related to ALS.

The majority of AEs in both treatment groups were related to ALS. Adverse events occurring in at least 10% of subjects in either treatment group were fall, muscular weakness, constipation, salivary hypersecretion, depression, dyspnoea, dysarthria, dysphagia, headache, dry mouth, oedema peripheral, upper respiratory tract infection, anxiety, sinusitis, cough, and muscle spasticity. No differences were observed between the 2 treatment groups in the incidence of AEs or in the incidence of vital sign, ECG, or laboratory abnormalities that met pre-specified criteria for potential clinical significance. One subject in the 300 mg group was discontinued during the double-blind treatment period due to neutropenia that was reported at the Part 1, Week 12 visit, baseline of the placebo washout period of Part 2.

The primary analysis of the treatment effect on the slope of ALSFRS-R total scores was not statistically significant; however, the estimated slope for the 300 mg group (−1.021) was improved by 20% relative to the estimated slope for the 50 mg group (−1.283). According to a recent survey of ALS-specialty physicians, a reduction of ALSFRS-R decline of 25% is considered to be clinically significant. The improvement in functional decline observed for the 300 mg group compared to the 50 mg group, therefore, was near a level that is considered by ALS-specialty physicians to be a clinically significant treatment effect. In addition, at each assessment between Weeks 32 and 52, mean decreases in ALSFRS-R total scores were less in the 300 mg group than in the 50 mg group.

To compare the global clinical outcomes between the 2 treatment groups, a joint-rank test (generalized Gehan Wilcoxon test) of survival and ALSFRS-R data was conducted. The results of this test demonstrated a statistically significant difference favoring the 300 mg group through Week 28 (p=0.046). As an alternative means of adjusting the functional analysis for the impact of death outcomes in each treatment group, the linear mixed-effects model for slopes of ALSFRS-R total scores was run on a dataset for which the first post-death score was imputed as 0 for subjects who died during the study. Because of the large imbalance in deaths during the randomized double-blind treatment period (in favor of the 300 mg group), the resulting impact on the slopes of the 2 groups was −2.05 in the 50 mg group versus −1.19 in the 300 mg group, a reduction in decline of 42% (p=0.018).

The mean change from baseline to Week 28 in upright vital capacity was −12.4% in the 50 mg group and −15.1% in the 300 mg group; median changes were −10.4% and −11.5%, respectively. The estimates of slope for vital capacity for the 30 mg and 300 mg groups over the double-blind treatment period through Week 28 were −2.452 and −3.067 (unadjusted), respectively, and −4.17 and −3.42 (adjusted for deaths through Week 28), respectively. For the adjusted vital capacity slopes, the 300 mg group slope was attenuated by 18% relative to the 50 mg group slope, demonstrating improvement in functional decline among subjects in the 300 mg group. No treatment effects on the McGill SIS scores were noted.

Results of this study demonstrate that dexpramipexole is safe and well tolerated in subjects with ALS up to a year of treatment at doses of 50 mg and 300 mg per day. The findings suggest that dexpramipexole may slow functional decline in ALS, as measured by the ALSFRS-R and/or vital capacity.

### Example 4

Safety, tolerability, and pharmacokineticsof dexpramipexole (dexpramipexole) in healthy adult subjects. Two Phase 1 clinical studies were conducted to assess the safety, tolerability, and pharmacokinetics (PK) of single and multiple doses of dexpramipexole in 54 healthy male and female adults. The effect of food on the single-dose PK of dexpramipexole was also evaluated. Single doses (50 mg, 150 mg, or 300 mg) and multiple doses (50 mg BID, 100 mg BID, or 150 mg BID) of dexpramipexole over 4.5 days were safe and well tolerated. Dexpramipexole was rapidly absorbed, with Tₘₐₓ ranging from 1.75 hours to 2.58 hours, t_{1/2} ranging from 6.40 hours to 8.05 hours under fasted conditions, and was mostly eliminated in urine as unchanged parent drug (84-90% of dose). Food had no effect on the single-dose PK of dexpramipexole. These findings support the ongoing development of dexpramipexole for the treatment of ALS and further evaluation of the compound's therapeutic potential in other neurodegenerative diseases.

A total of 54 subjects (30 subjects in Study CL001 and 24 subjects in Study CL002) were enrolled. Healthy, non-smoking, male and female subjects 30 to 60 years of age, inclusive, with normal or clinically acceptable physical examination and electrocardiogram (ECG) findings, systolic (90 to 140 mmHg) and diastolic (50 to 90 mmHg) blood pressure, and resting heart rate (50 to 100 bpm) who were willing to provide signed, written informed consent were eligible for enrollment. Female volunteers had to be of non-childbearing potential with negative pregnancy test results at screening and clinic check-in. Subjects with any history of neurodegenerative illnesses were excluded. The use of over-the-counter medications within 7 days prior to enrollment or prescription medications within 12 weeks prior to enrollment was prohibited. Subjects with prior exposure to dexpramipexole, to any other drug product containing dexpramipexole, or to any dopamine agonist, including pramipexole, were excluded.

Both studies were randomized, double-blind, placebo-controlled, ascending dose, single-center studies designed to evaluate the safety, tolerability, and PK of dexpramipexole. In the first study, subjects were enrolled in 3 successive double-blind, placebo-controlled panels of 8 subjects (n=6 active, n=2 placebo per panel). Following the completion of the third panel, an additional panel of 6 subjects was enrolled to conduct a preliminary evaluation of the effect of food on absorption of dexpramipexole. Subjects were randomized to receive dexpramipexole 50 mg or placebo in Panel 1, dexpramipexole 150 mg or placebo in Panel 2, and dexpramipexole 300 mg or placebo in Panel 3. Subjects in Panel 4 received a single dose of dexpramipexole 150 mg 30 minutes after beginning a standard high-fat/high-calorie breakfast.

In the second study, subjects were enrolled in 3 successive double-blind, placebo-controlled, panels of 8 subjects (n=6 active, n=2 placebo per panel). Subjects in all panels were randomized to receive a single dose of active drug or placebo on Day 1, after which they began a dosing regimen twice daily (every 12 hours) beginning in the morning of Day 3. Panel 1 randomized subjects received either dexpramipexole 50 mg or placebo on Day 1, followed by 50 mg or placebo doses twice daily on Day 3 through Day 6 with a final dose on the morning of Day 7. The same dosing schedule was applied to subjects randomized in Panel 2 (dexpramipexole 100 mg twice daily) and Panel 3 (dexpramipexole 150 mg twice daily).

In both studies, dexpramipexole (>99.95% enantiomeric purity) was supplied as neat drug substance (no excipients) in hard gelatin capsules. Matching placebo capsules contained equivalent weights of microcrystalline cellulose. Capsules were administered orally with water. A purity adjustment factor of 1.06 was used to adjust for the water weight (monohydrate) in the salt form of the dexpramipexole drug substance. Subjects in the fasted cohorts were required to fast overnight for a minimum of 10 hours before dose administration. Subjects in the food cohort were required to fast overnight for a minimum of 10 hours before dose administration, with exception of the high fat/high calorie meal that was administered 30 minutes prior to drug administration. Panels of ascending doses were enrolled sequentially, with at least 96 hours and 72 hours separating the initiation of each panel in the single-dose and multiple-dose studies, respectively. All available safety data were reviewed under blinded conditions to monitor for serious safety or tolerability events prior to proceeding with dose escalations.

In the first study, blood samples to measure plasma concentrations of dexpramipexole were obtained pre-dose (0 hour), at 15, 30, and 45 minutes post-dose, and at 1, 1.5, 2, 2.5, 3, 4, 6, 8, 12, 16, 24, 36, 48, and 72 hours post-dose. Urine samples for the analysis of dexpramipexole concentrations were obtained before dosing and at pooled intervals of 0-2, 2-4, 4-8, 8-12, 12-24, 24-36, 36-48, and 48-72 hours after dosing.

In second study, blood samples to measure plasma concentrations of dexpramipexole were obtained on Day 1 and Day 7 at pre-dose (0 hour), at 15, 30, and 45 minutes post-dose, at 1, 1.5, 2, 2.5, 3, 4, 6, 8, 12, 16, 24, 36, and 48 hours post-dose, prior to the morning dose on Days 5 and 6, and on Day 10 at 72 hours after the final dose. Urine samples for PK testing were collected pre-dose (0 hour) on Day 7 and during the following post-dose intervals: 0-2, 2-4, 4-6, 6-8, 8-10, and 10-12 hours. In both studies, a complete collection was attempted for each urine sample interval.

Blood samples were collected into a 10 mL dipotassium ethylenediaminetetraacetic acid (K₂-EDTA) Vacutainer® via an indwelling peripheral intravenous cannula or by direct venipuncture. Within 15 minutes of collection, the samples were centrifuged at 3000 rpm for 10 minutes at 4° C. After centrifugation, the plasma was divided into 2 aliquots of at least 1.5 mL each, placed into polypropylene containers, frozen, and stored at −20° C. until they were shipped for analysis.

Urine collected in each interval was well mixed, the pH was recorded, the total volume (or the weight and specific gravity) was recorded, and 2 aliquots of 20 mL each were collected into polypropylene containers and stored at −20° C. until they were shipped for analysis. All plasma and urine sample were shipped frozen on dry ice in 2 separate shipments per group (1 set of aliquots per shipment) to Eurofins AvTech Laboratories Inc. (Kalamazoo, Mich.) for bioanalytical analysis.

Plasma and urine concentrations were measured using validated liquid chromatography/mass spectrometry/mass spectrometry (LC/MS/MS) methods. For the first study, the lower limits of quantitation for dexpramipexole were 20 ng/mL in plasma and 0.1 μg/mL in urine. The inter- and intra-day coefficients of variation (CV) were 7% to 8% and 1% to 17%, respectively, for plasma. The corresponding CVs for urine were 5% to 7% and 0% to 7%. For the second study, the lower limits of quantitation for dexpramipexole were 2 ng/mL in plasma and 0.1 μg/mL in urine. The inter- and intra-day coefficients of variation (CV) were 5% to 11% and 1% to 8%, respectively, for plasma and 6% to 10% and 0% to 7%, respectively, for urine. The analytical procedure for analysis of plasma samples used a 100 μL aliquot of K₂EDTA human plasma. The plasma sample was spiked with 20 μL of working internal standard solution and 20 μL of type 1 water for subject samples and QCs and 20 μL of the appropriate intermediate standard solution for standards. One hundred microliters (100 μL) of 50% ammonium hydroxide solution was added to the sample followed by vortex mixing. One milliliter (1 mL) of tertbutyl methyl ether was then added and the sample was vortexed to extract the analyte and internal standard into the organic layer, followed by separation using flash freezing. The organic layer was decanted, evaporated to dryness, and the sample was reconstituted with 0.5 mL of reconstitution solution (0.1% ammonium hydroxide in 50:50 methanol; type 1 water (v/v/v)). A 10 μL aliquot of this reconstituted sample was injected into an LC/MS/MS system for analysis. The MS/MS transitions monitored were 212.1 m/z to 153.1 m/z for dexpramipexole and 219.2 m/z to 111.2 m/z for the internal standard, D7-pramipexole. The calibration curve was linear between 2 and 2,000 ng/mL for dexpramipexole using a weighted (1/concentration) linear regression of the standard curve. The analytical procedure for analysis of urine samples was essentially similar to the plasma procedure.

For both studies, the following PK parameters were estimated from individual plasma and concentration data using non-compartmental analysis: maximum plasma concentration (Cₘₐₓ), time to Cₘₐₓ (Tₘₐₓ), area under the curve from time zero to the final time with a concentration above the limit of quantitation (AUC₀₋ₜ), area under the curve from zero to infinity (AUC_{inf}), area under the curve over the dosing interval on Day 7 (AUC₀₋₁₂) for the multiple-dose study, elimination rate constant (λz), half-life (t_{1/2}), amount excreted in the urine (Ue), fraction excreted unchanged in urine (Fe), renal clearance (Clr), oral clearance (CL/F), and oral volume of distribution (Vz/F). Plasma concentrations, urinary excretions, and PK parameters were summarized by dose level using descriptive statistics.

In both studies, safety was assessed by periodic measurement of vital signs, 12-lead ECGs, physical examinations, clinical laboratory parameters, and reports of adverse events. Baseline vital signs and changes from baseline were summarized with descriptive statistics by body position (supine or standing), dose level, and time point. Additionally, the number of subjects with substantial increases or decreases in blood pressure (>20 mmHg) and heart rate (>15 bpm) were tabulated by dose level and visit. The arithmetic mean of 3 readings of blood pressure and heart rate at each visit/position was used for the analysis. The shift from baseline for physical examination results was tabulated by body system, visit, and dose level. The overall ECG findings were summarized using a shift table comparing post-baseline visits to baseline. Laboratory parameters were summarized at each timepoint, including changes from baseline by dose level. In addition, abnormal values outside normal ranges were flagged. All safety data were summarized with descriptive statistics by dose group.

In the first study, subjects remained in the clinic for 72 hours after dosing, during which time they were monitored for safety and tolerability, and later returned to the clinic for a brief follow-up visit 7 days post-dose for clinical and laboratory assessments. In the second study, end-of-treatment evaluations were performed on Day 9, approximately 48 hours after the final dose, and subjects were discharged from the clinic for an outpatient visit on Day 10 and a follow-up visit on Day 14.

In both studies, analysis of the plasma and urine concentration data for dexpramipexole after oral administration indicated rapid absorption and linear PK over all doses and time intervals tested. Mean values for Cₘₐₓ, AUC₀₋ₜ, and AUC_{inf} increased in a dose-proportional manner. Mean Tₘₐₓ, which ranged from 1.75 hours to 2.58 hours, and t_{1/2}, which ranged from 6.40 hours to 8.05 hours under fasted conditions, were independent of dose.

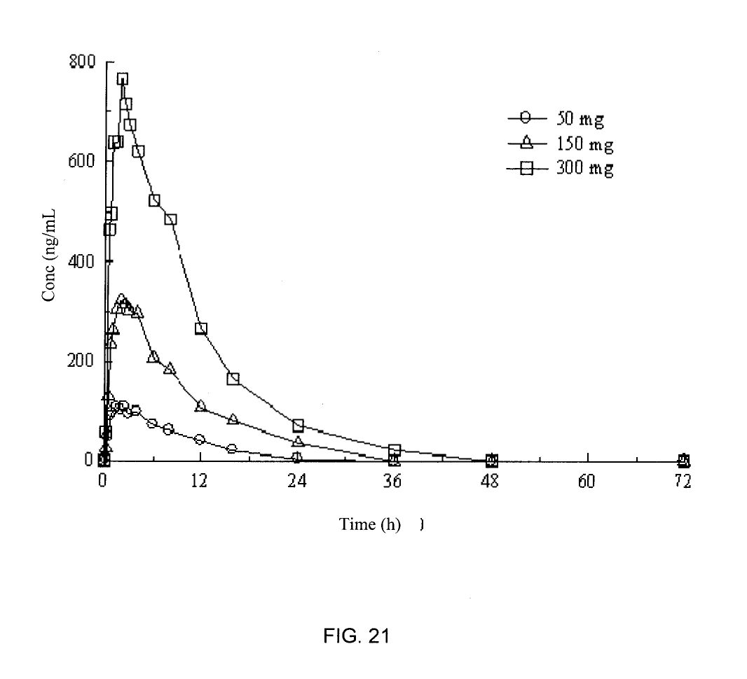

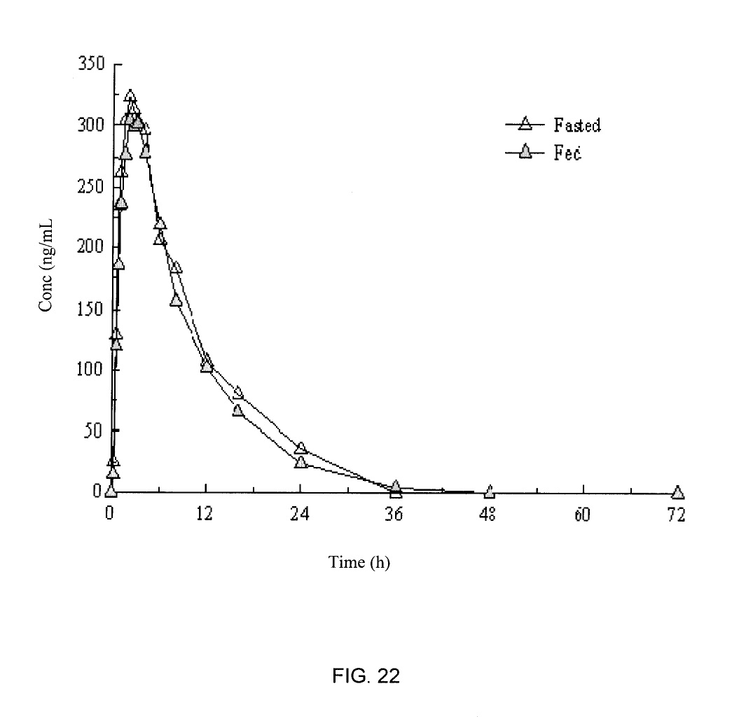

A summary of PK parameters in the first study is presented in TABLE 19. Oral administration of dexpramipexole 50 mg, 150 mg, and 300 mg indicated linear PK over the dose range (FIG. 21). The elimination t_{1/2} ranged from 6.40 hours to 6.96 hours under fasted conditions. Approximately 90% of the dose was recovered as unchanged parent drug in the urine, and renal clearance was 4 to 5 times greater than glomerular filtration, which is consistent with active secretion. Food did not affect the absorption or elimination of dexpramipexole (FIG. 22).

TABLE 19
Summary of Pharmacokinetic Parameters for Dexpramipexole after Oral
Administration of Single 50 mg, 150 mg, and 300 mg Single Doses to
Adult Subjects under Fasted Conditions and 150 mg under Fed
Conditions

| | Dexpramipexole (Fasted) | | Dexpramipexole (Fed) | |
|---|---|---|---|---|
| | 50 mg | 150 mg | 300 mg | Fed 150 mg |
| | (N = 6) | (N = 6) | (N = 6) | (N = 6) |
| PK Parameter | Mean ± SD | Mean ± SD | Mean ± SD | Mean ± SD |
| Cmax (ng/mL) | 125 ± 22.0 | 360 ± 60.4 | 781 ± 158 | 315 ± 61.6 |
| Tmax (h)^{a} | 2.04 | 2.04 | 1.98 | 2.58 |
| AUC₀₋ₜ, (h*ng/mL) | 989 ± 295 | 3360 ± 780 | 8340 ± 3203 | 3080 ± 934 |
| | | (N = 5) | | |
| AUC_{inf} (h*ng/mL) | 1254 ± 347 | 3782 ± 1012 | 8624 ± 3263 | 3379 ± 957 |
| | | (N = 5) | | |
| λz (h⁻¹) | 0.1064 ± 0.0171 | 0.1001 ± 0.0087 | 0.1151 ± 0.0309 | 0.1144 ± 0.0259 |
| | | (N = 5) | | |
| t_{1/2} (h) | 6.65 ± 1.07 | 6.96 ± 0.56 | 6.40 ± 1.73 | 6.33 ± 1.49 |
| | | (N = 5) | | |
| CL/F (mL/min) | 527 ± 135 | 524 ± 146 | 492 ± 194 | 581 ± 127 |
| | | (N = 5) | | |
| Vz/F (L) | 294 ± 46.2 | 311 ± 68.4 | 258 ± 73.5 | 308 ± 55.9 |
| Ue (mg) | 35.3 ± 5.19 | 74.8 ± 50.17 | 198 ± 28.0 | 96.9 ± 4.71 |
| Fe (% dose) | 94.7 ± 13.9 | 66.9 ± 44.8 | 88.3 ± 12.5 | 86.6 ± 4.21 |
| CLr (mL/min) | 628 ± 149 | 385 ± 236.5 | 441 ± 159 | 559 ± 140 |

PK = pharmacokinetics; SD = standard deviation
^{a}Median, rather than mean ± SD, reported for Tmax

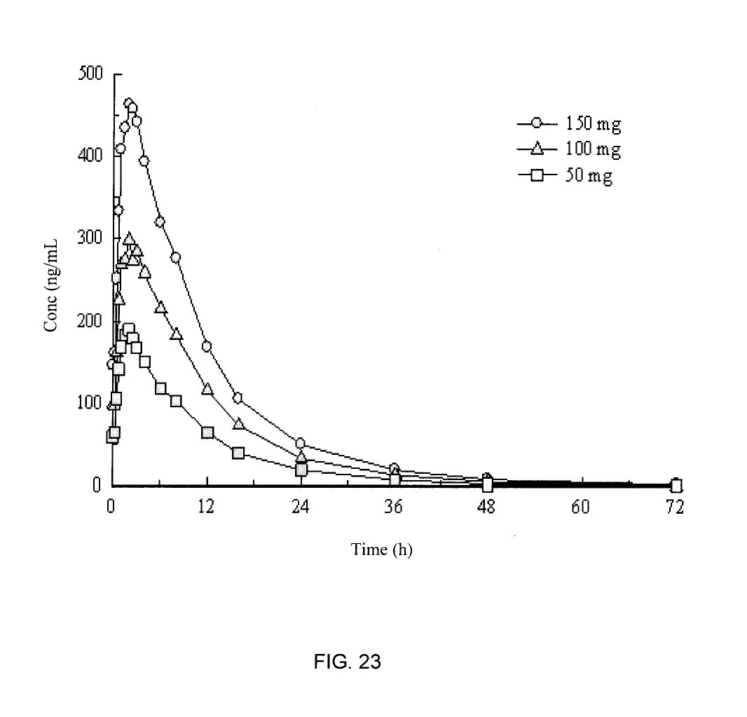

A summary of PK parameters on Day 7 of second study is presented in Table 20. Oral administration of single dexpramipexole 50 mg, 100 mg, and 150 mg doses on Day 1, twice daily doses on Days 3 through 6, and a single dose on Day 7 indicated linear PK over the dose range (FIG. 23). The accumulation of dexpramipexole at 1.2-fold to 1.4-fold was consistent with the t_{1/2} and dosing interval and further supported the linearity of the PK. The steady-state elimination t_{1/2} (Day 7) under fasted conditions ranged from 6.87 hours to 8.05 hours and approximately 84% of the dose was recovered in the urine as unchanged parent drug over a 12-hour steady-state dosing period. Renal clearance was greater than the glomerular filtration rate, again consistent with active secretion, and did not appear to be saturated at the doses administered.

TABLE 20
Summary of Pharmacokinetic Parameters for Dexpramipexole on Day 7
after Oral Administration of 50 mg, 100 mg, and 150 mg Single Doses on
Day 1, Twice Daily Doses on Day 3 through 6, and Single Doses on Day 7
under Fasted Conditions

| | Dexpramipexole (Fasted) | | |
|---|---|---|---|
| | 50 mg Twice Daily | 100 mg Twice Daily | 150 mg Twice Daily |
| | (N = 6) | (N = 6) | (N = 6) |
| PK Parameter | Mean ± SD | Mean ± SD | Mean ± SD |
| Cmax (ng/mL) | 191 ± 20.9 | 306 ± 54.8 | 479 ± 74.6 |
| Tmax (h)^{a} | 1.75 | 2.02 | 2.18 |
| AUC(0-12) (h*ng/mL) | 1449 ± 221 | 2467 ± 304 | 3749 ± 575 |
| λz (h⁻¹) | 0.1039 ± 0.0193 | 0.0893 ± 0.0117 | 0.0895 ± 0.0184 |
| t_{1/2} (h) | 6.87 ± 1.29 | 7.89 ± 1.19 | 8.05 ± 1.80 |
| CL/F (mL/min) | 437 ± 60.8 | 510 ± 57.4 | 507 ± 74.1 |
| Vz/F (L) | 255 ± 24.0 | 348 ± 61.7 | 349 ± 75.8 |
| | | (n = 4) | (n = 5) |
| Ue (mg) | 32.5 ± 3.82 | 56.3 ± 2.62 | 100.3 ± 8.76 |
| | | (n = 4) | (n = 5) |
| Fe (% dose) | 87.2 ± 10.26 | 75.5 ± 3.51 | 89.6 ± 7.83 |
| | | (n = 4) | (n = 5) |
| CLr (mL/min) | 385 ± 90.3 | 382 ± 62.7 | 451 ± 102.2 |

PK = pharmacokinetic; SD = standard deviation
^{a}Median, rather than mean ± SD, reported for Tmax

No serious adverse events or adverse events that led to early discontinuation occurred in either study. The adverse event profile in each active dose group was similar to that in the placebo group. The most frequently reported adverse event was dizziness (3 placebo subjects, 3 dexpramipexole subjects) in the first study and headache (1 placebo subject, 5 dexpramipexole subjects) in the second study. All adverse events were mild in intensity except for 1 subject in the dexpramipexole 150 mg group of the first study, who reported moderate nausea and vomiting and a severe headache on the day of dosing.

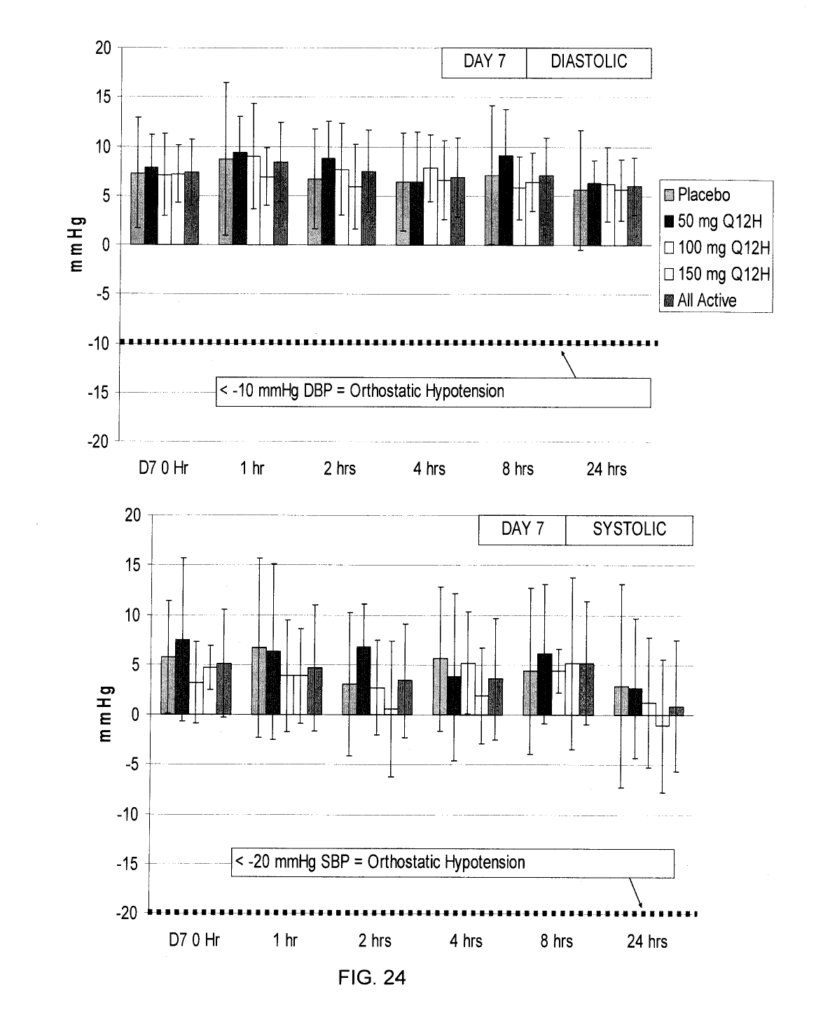

There was no evidence of an overall drug effect or a dose-dependent drug effect on vital signs (supine or standing blood pressure and heart rate, postural change in blood pressure or heart rate), physical examinations, ECG assessments, or hematology and urinalysis parameters in either study. The absence of effects of oral administration of dexpramipexole on the difference between supine and standing blood pressures on Day 7 of Study CL002 are shown in FIG. 24. In the first study, potentially clinically significant elevations in triglycerides were reported in 2 dexpramipexole subjects on Day 7; however, both subjects had elevated triglycerides at baseline. One of these subjects also had a potentially clinically significant elevation in serum creatinine on Day 7 that returned to within normal range upon repeat testing on Day 19.

The unmet medical need in the treatment of ALS is very high and new effective treatments are urgently needed. Dexpramipexole, administered as a highly chirally pure drug substance, is a promising novel amino-benzothiazole that is being developed for the treatment of ALS. Preclinical studies have shown that dexpramipexole and its enantiomer pramipexole are equally neuroprotective, but, unlike pramipexole, dexpramipexole is not a clinically relevant dopamine agonist, and therefore may be dosed at much higher levels that may optimize its neuroprotective properties in the absence of dose-limiting side effects. The proposed mechanisms of action of pramipexole that may lead to neuroprotection involve antiapoptotic, antioxidant, and antitoxic mechanisms, as well as induction of neurotrophic factors. While these may also be pharmacodynamically relevant properties of dexpramipexole, recent studies have importantly shown that dexpramipexole increases bioenergetic efficiency in stressed mitochondria.

In the present studies, oral administration of dexpramipexole in single doses up to 300 mg and multiple doses up to 150 mg twice daily for 4½ days was safe and well tolerated. There was no evidence of clinically significant effects of dexpramipexole on heart rate or blood pressure and no evidence of orthostatic hypotension was observed. Specifically, there were no dopaminergic-related, dose-limiting side effects observed following single doses of dexpramipexole up to 300 mg and multiple doses of up to 150 mg twice daily.

Dexpramipexole was well absorbed after oral administration, with maximum concentrations observed 2 hours after dosing. Dexpramipexole demonstrated linear PK over the range of doses studied and was nearly completely eliminated in the urine as unchanged parent drug (84-90% of dose). Single-dose absorption was not affected by administration of a high fat/high calorie meal.

Although not a specific objective of these Phase 1 studies, it was determined that dexpramipexole, at the doses examined, lacks clinically relevant dopaminergic activity, in marked contrast to its enantiomer, pramipexole. The highest unit dose of dexpramipexole administered in these studies (300 mg) was 2400-fold higher than the recommended safe starting unit dose of pramipexole (0.125 mg) and 67-fold higher than the maximum recommended daily dose (4.5 mg/day) of pramipexole in Parkinson's disease patients, a dose of pramipexole which may only be reached following a seven-week period of gradual dose titration.

The PK and safety results from these 2 Phase 1 clinical studies support continued development of dexpramipexole as a treatment of ALS and potentially other neurodegenerative diseases.

What is claimed is:

1. A method for treating amyotrophic lateral sclerosis (ALS) in a patient comprising:
administering to the patient an effective amount of about chirally pure (6R)-2-amino-4,5,6,7-tetrahydro-6-(propylamino)benzothiazole or pharmaceutically acceptable salt thereof.

2. The method of claim 1, wherein treating comprises slowing progression of amyotrophic lateral sclerosis (ALS), reducing intensity of symptoms associated with amyotrophic lateral sclerosis (ALS), reducing onset of symptoms associated with amyotrophic lateral sclerosis (ALS), reducing weight loss associated with amyotrophic lateral sclerosis (ALS), reversing weight loss associated with amyotrophic lateral sclerosis (ALS), delaying mortality, and combinations thereof.

3. The method of claim 2, wherein the symptoms associated with amyotrophic lateral sclerosis (ALS) are selected from group consisting of fine motor function, gross motor function, balbar function, respiratory function, and combinations thereof.

4. The method of claim 2, wherein the symptoms associated with amyotrophic lateral sclerosis (ALS) are selected from the group consisting of walking, speech, eating, swallowing, writing, climbing stairs, cutting food, turning in bed, salivation, dressing, maintaining hygiene, breathing, dyspnea, orthopnea, respiratory insufficiency, and combinations thereof.

5. The method of claim 1, wherein the effective amount is from about 50 mg to about 300 mg per day.

6. The method of claim 1, wherein the effective amount is from about 150 mg to about 300 mg per day.

7. The method of claim 1, wherein the effective amount is about 300 mg or more per day.

8. The method of claim 1, wherein administering comprises administering a dose equal to about half of the daily dose two times per day.

9. The method of claim 1, wherein administering comprises administering a dose equal to about half of a daily dose every 12 hours.

10. The method of claim 1, wherein administering comprises administering a dose equal to about one quarter of a daily dose four times per day.

11. The method of claim 1, wherein administering comprises administering about 150 mg two times per day.

12. The method of claim 1, wherein administering comprises administering about 75 mg four times per day.

13. The method of claim 1, wherein the method is carried out for a time period selected from the group consisting of at least about 12 weeks, at least about 6 months, at least about 1 year, at least about 2 years, at least about 3 years, at least about 4 years, at least about 5 years, at least about 10 years, and until the patient dies.

14. The method of claim 1, wherein the method is carried out at least daily for an indefinite amount of time.

15. The method of claim 1, further comprising administering one or more other ALS treatments simultaneously or concurrently with administering about chirally pure (6R)-2-amino-4,5,6,7-tetrahydro-6-(propylamino)benzothiazole or a pharmaceutically acceptable salt thereof.

16. The method of claim 15, wherein the one or more other ALS treatment includes riluzole.

17. The method of claim 1, wherein the patient began exhibiting symptoms of ALS less than about two years before beginning administering of (6R)-2-amino-4,5,6,7-tetrahydro-6-(propylamino)benzothiazole or pharmaceutically acceptable salt thereof.

18. The method of claim 1, wherein the patient began exhibiting symptoms of ALS at least greater than about two years before beginning administering of (6R)-2-amino-4,5,6,7-tetrahydro-6-(propylamino)benzothiazole or pharmaceutically acceptable salt thereof.

19. The method of claim 1, wherein the patient exhibits a greater than 20 % improvement in ALS Functional Rating Scale, Revised (ALSFRS-R) score when compared to baseline.

20. The method of claim 1, wherein the patient exhibits a greater than 30% improvement in ALS Functional Rating Scale, Revised (ALSFRS-R) score when compared to baseline.

21. The method of any one of claims 25 or 26, wherein the improvement is apparent in a time period selected from the group consisting of less than about 9 months, less than about 6 months, less than about 3 months, and less than about 1 month.

22. The method of claim 1, wherein administering about chirally pure (6R)-2-amino-4,5,6,7-tetrahydro-6-(propylamino)benzothiazole or pharmaceutically acceptable salt thereof results in slowing of a rate of fine motor function loss in the patient.

23. The method of claim 1, further comprising administering a daily dose of greater than an effective amount for a period of time before administering an effective amount of about chirally pure (6R)-2-amino-4,5,6,7-tetrahydro-6-(propylamino)benzothiazole or pharmaceutically acceptable salt thereof.

24. The method of claim 23, wherein the greater than an effective amount is greater than 150 mg.

25. The method of claim 23, wherein the greater than an effective amount is greater than 300 mg.

26. The method of claim 23, wherein the period of time before administering an effective amount is from about 1 weeks to about 12 weeks.

27. The method of claim 23, wherein the period of time before administering an effective amount is from about 2 weeks to about 6 weeks.

28. The method of claim 23, wherein administering an effective amount of about chirally pure (6R)-2-amino-4,5,6,7-tetrahydro-6-(propylamino)benzothiazole or pharmaceutically acceptable salt thereof is carried out indefinitely.

29. The method of claim 1, wherein an effective amount of about chirally pure (6R)-2-amino-4,5,6,7-tetrahydro-6-(propylamino)benzothiazole or pharmaceutically acceptable salt thereof is administered in an initial dose and every administration thereafter.

30. The method of claim 1, wherein dosing achieves a dose dependent, steady state AUC₀₋₁₂ (h×ng/mL) selected from the group consisting of 836±234 for an effective amount of 50 mg, 2803±1635 for an effective amount of 150 mg, and 6004±2700 for an effective amount of 300 mg.

31. The method of claim 1, wherein the effective amount comprises a stable daily dose.

32. The method of claim 31, wherein the stable daily dose comprises from about 50 mg to about 300 mg of about chirally pure (6R)-2-amino-4,5,6,7-tetrahydro-6-(propylamino)benzothiazole or pharmaceutically acceptable salt thereof.

33. The method of claim 31, wherein the stable daily dose comprises 1 to 5 unit doses per day.

34. The method of claim 33, wherein each unit dose is a solid unit dose.

35. The method of claim 31, wherein administering comprises administering one unit dose two times per day wherein each unit dose is equal to about half of the stable daily dose.

36. The method of claim 31, wherein administering comprises administering one unit dose once every 12 hours wherein each unit dose is equal to about half of the stable daily dose.

37. The method of claim 31, wherein administering comprises administering one unit dose four times per day wherein each unit dose is equal to about one quarter of the stable daily dose.

38. The method of claim 31, wherein administering comprises administering two unit doses wherein each unit dose is about 150 mg two times per day.

39. The method of claim 31, wherein administering comprises administering four unit doses wherein each unit dose is about 75 mg four times per day.

40. The method of claim 31, wherein administering a stable daily dose of about chirally pure (6R)-2-amino-4,5,6,7-tetrahydro-6-(propylamino)benzothiazole or pharmaceutically acceptable salt thereof is carried out for at least about 12 weeks.

41. The method of claim 39, wherein administering a stable daily dose of about chirally pure (6R)-2-amino-4,5,6,7-tetrahydro-6-(propylamino)benzothiazole or pharmaceutically acceptable salt thereof is carried out for an indefinite amount of time.

42. The method of claim 31, wherein the stable daily dose is consistent throughout a treatment regimen.

43. The method of claim 31, wherein an initial daily dose is equal to each daily dose thereafter.

44. The method of claim 31, wherein there is not titration before administering the stable daily dose.

45. The method of claim 31, wherein administering achieves a dose dependent, steady state AUC₀₋₁₂ (h×ng/mL) selected from 836±234 for stable daily dose of 50 mg, 2803±1635 for stable daily dose of 150 mg, or 6004±2700 for stable daily dose of 300 mg.

46. The method of claim 1, further comprising monitoring the patient.

47. The method of claim 1, further comprising monitoring the patient for neutropenia.

48. The method of claim 1, further comprising monitoring ALSFRS-R score for the patient.

49. The method of claim 1, further comprising monitoring the patients fine motor function, gross motor function, bulbar function, respiratory function, and combinations thereof.

50. The method of claim 1, further comprising monitoring behaviors selected from the group consisting of swallowing, handwriting, speech, ability to walk, ability to climb stairs, ability to dress, ability to maintain hygiene, and combinations thereof.

51. The method of claim 1, further comprising scheduling a doctor visit every 6 months for at least 12 months.

52. The method of claim 1, wherein the patient is predisposed to amyotrophic lateral sclerosis (ALS) and is not exhibiting symptoms of amyotrophic lateral sclerosis (ALS).

53. The method of claim 1, further comprising administering about chirally pure (6R)-2-amino-4,5,6,7-tetrahydro-6-(propylamino)benzothiazole or pharmaceutically acceptable salt thereof to family members of the patient.

54. The method of claim 1, wherein treating comprises administering the about chirally pure (6R)-2-amino-4,5,6,7-tetrahydro-6-(propylamino)benzothiazole or pharmaceutically acceptable salt thereof to a patient not exhibiting symptoms of amyotrophic lateral sclerosis (ALS).

55. The method of claim 1, wherein treating comprises administering the about chirally pure (6R)-2-amino-4,5,6,7-tetrahydro-6-(propylamino)benzothiazole or pharmaceutically acceptable salt thereof to a patient that is predisposed to amyotrophic lateral sclerosis (ALS).
